(19) United States
(12) Patent Application Publication
(10) Pub. No.: US 2020/0399632 A1
(43) Pub. Date: Dec. 24, 2020

(54) SENSOR SYSTEMS

(71) Applicant: enEvolv, Inc., Medford, MA (US)

(72) Inventors: Matthew R. DUNN, Medford, MA (US); Kristin J. ADOLFSEN, Medford, MA (US); Noah D. TAYLOR, Medford, MA (US); James E. SPOONAMORE, Medford, MA (US); Jay H. KONIECZKA, Medford, MA (US); Caitlin D. ALLEN, Medford, MA (US); Lindong WENG, Medford, MA (US)

(21) Appl. No.: 16/970,132

(22) PCT Filed: Feb. 15, 2019
(86) PCT No.: PCT/US2019/018273

§ 371 Date: Aug. 14, 2020

Related U.S. Application Data
(60) Provisional application No. 62/730,355, filed on Sep. 12, 2018.
(60) Provisional application No. 62/631,090, filed on Feb. 15, 2018.

(51) Int. Cl. C12N 15/10

(52) U.S. Cl. CPC C12N 15/1058; C12N 15/1086

(57) ABSTRACT

The present technology relates to methods and compositions that provide for improved detection of target molecules in, for example, bioengineering.

### CROSS-REFERENCE TO RELATED APPLICATIONS

This application claims the benefit of U.S. Provisional Application No. 62/631,090, filed Feb. 15, 2018, and U.S. Provisional Application No. 62/730,355, filed Sep. 12, 2018, the contents of which are hereby incorporated by reference herein in their entirety.

### GOVERNMENT INTEREST

This invention was made with government support under Grant Nos. D16PC00132 awarded by the Defense Advanced Research Projects Agency (DARPA). The government has certain rights in the invention.

### FIELD

The present technology relates to methods and compositions for detecting and enriching engineered product producing cells using engineered protein sensors.

### BACKGROUND

The use of bacterial allosteric transcription factors (aTFs)—single proteins that directly couple the recognition of a small molecule to a transcriptional output—has been proposed for use in metabolic engineering strategies to improve enzymatic bioproduction and detection (Taylor, et al. Nat. Methods 13(2): 177). The protein's conformational change caused by effector binding modulates its affinity for a specific operator DNA sequence, which alters gene expression by up to 5000-fold. This makes aTF sensors an exciting paradigm to address the sense-and-respond challenge that is central to many applications of synthetic biology.

aTFs rapidly sense ligands and elicit targeted transcriptional changes, such as the induced expression of a reporter (e.g., fluorescent protein or selection marker). This allows for the enrichment of cells with a high intracellular concentration of the cognate ligand (e.g., by fluorescence activated cell sorting (FACS) or growth). In the context of metabolic engineering, this greatly increases the throughput at which engineered strains can be screened.

However, in certain genomic engineering situations (e.g., when doing large-scale genomic manipulations that target core metabolic processes), engineered sensors may not be reliably deployed due to sensor performance variation. Sensor response variation across mutants has been seen, such as in a chemically mutagenized library of E. coli mutants screened with a plasmid-based lysine sensor system (Binder et al., Genome Biology, 13(R40), 1-12, 2012).

Sensor performance may be restored by decoupling the production and sensing functions. For example, co-culturing two strains together where one strain is dedicated to production and the second to sensing allows for the genomic variation to modify the production levels while each unmodified sensor strain provides a robust response to product levels generated by the producer. However, to do this, each of the engineered producer strains being screened must be grown with the sensor strain in a unique growth vessel, which presents a challenge when using producer strain libraries with greater than 10⁶ unique members.

In other situations, (e.g., in the cases when working with diffusible or actively exported products), engineered producer strains must be grown and screened in isolation to avoid crosstalk of nonproducers with better producers in the population. For example, co-culturing two strains of producers that either produce a high or low amount of naringenin and are transformed with the GFP-based naringenin sensor system should produce two subpopulations that demonstrate high and low GFP-based fluorescence after a production phase. However, after production, only one intermediately fluorescent population is seen suggesting response to the bulk level of diffused product throughout the entire population rather than each cell's individual production total (Example 2, FIG. 3). In an actual selection, this population averaging or crosstalk would prevent the researcher's ability to identify engineered strains with higher production from the rest of the population. Overcoming this challenge additionally requires the unique compartmentalization of each engineered strain within its own growth vessel.

Accordingly, there is a need for improved methods and systems for detecting and enriching engineered product producing cells.

### SUMMARY

The present technology provides methods and compositions for the improved growth and selection of engineered producer strains (i.e., cells) within either bulk-mixed or microfluidically-generated droplets using engineered sensor technology. In some embodiments, microfluidically generated droplets provide uniform and isolated growth vessels for engineered strains in a large scale. In some embodiments, droplets are generated at <20 kHz (e.g. less than about 20 kHz, or less than about 15 kHz, or less than about 10 kHz, or less than about 5 kHz) allowing for the encapsulation of 2,000 to 5,000 (e.g. about 2,000, or about 2,500, or about 3,000, or about 3,500, or about 4,000, or about 4,500, or about 5,000) unique producer strains every second, which enables the screening of <500 million unique producer strains per microfluidic device per day.

In one aspect, the present invention relates to a method for producing a population of engineered producer cells comprising: encapsulating each producer cell from a pool of genetically varied producer cells in a droplet to form a plurality of droplets encapsulating engineered producer cells; assaying the droplets for levels of a target molecule, wherein an engineered protein-based sensor provides a readout of the level of the desired target molecule produced by the producer cell through activation or repression of a reporter; isolating droplets with producer cells that produce desired levels of the target molecule; and, recovering the cells that produce desired levels of the target molecule to form the population of producer cells, wherein the population of producer cells is an enriched population that produce desired levels of the target molecule.

In another aspect, the present invention relates to a method for producing a population of engineered producer cells comprising: transforming a pool of engineered producer cells with an engineered sensor plasmid, wherein the engineered sensor plasmid encodes an engineered protein sensor; encapsulating each producer cell from the pool of genetically varied producer cells in a droplet to form a plurality of droplets encapsulating engineered producer cells; assaying the droplets for levels of a target molecule, wherein an engineered protein-based sensor provides a readout of the level of the target molecule produced by the producer cell through activation or repression of a reporter; isolating the droplets with producer cells that produce desired levels of the target molecule; and, recovering the cells that produce desired levels of the target molecule to form the population of producer cells, wherein the population of producer cells is an enriched population that produce desired levels of the target molecule. In another aspect, the present invention relates to a method for producing a population of engineered producer cells comprising: encapsulating each producer cell from the pool of genetically varied producer cells in a droplet to form a plurality of droplets encapsulating engineered producer cells; merging each droplet containing the producer cell with a droplet encapsulating an engineered-protein based sensor cell, wherein the engineered sensor cell produces an engineered protein sensor; assaying the droplets for levels of a target molecule, wherein an engineered protein-based sensor provides a readout of the level of the desired target molecule produced by the producer cell through activation or repression of a reporter; sorting the merged droplets to isolate droplets containing producer cells that produce desired levels of the target molecule; and, recovering the cells that produce desired levels of the target molecule to form the population of producer cells, wherein the population of producer cells is an enriched population that produce desired levels of the target molecule.

In another aspect, the present invention relates to a method for producing a population of engineered producer cells comprising: transforming a pool of engineered producer cells with an engineered sensor plasmid, wherein the engineered sensor plasmid encodes an engineered protein sensor; encapsulating each producer cell from a pool of genetically varied producer cells in a droplet to form a plurality of droplets encapsulating engineered producer cells; wherein each droplet is surrounded by an immiscible continuous phase that comprises a fluorinated-based oil or emulsion; assaying the droplets for levels of a target molecule, wherein an engineered protein-based sensor provides a readout of the level of the desired target molecule produced by the producer cell through activation or repression of a reporter; isolating droplets with producer cells that produce desired levels of the target molecule; recovering the cells that produce desired levels of the target molecule to form the population of producer cells, wherein the population of producer cells is an enriched population that produce desired levels of the target molecule.

In another aspect, the present invention relates to a method for producing a population of engineered producer cells comprising: encapsulating each producer cell from the pool of genetically varied producer cells in a droplet to form a plurality of droplets encapsulating engineered producer cells; wherein each droplet is: (a) surrounded by an immiscible continuous phase that comprises a fluorinated-based oil or emulsion, and (b) comprises an engineered sensor cell, wherein the engineered sensor cell produces an engineered protein sensor; assaying the droplets for levels of a target molecule, wherein an engineered protein-based sensor provides a readout of the level of the target molecule produced by the producer cell through activation or repression of a reporter; isolating the droplets with producer cells that produce desired levels of the target molecule; recovering the cells that produce desired levels of the target molecule to form the population of producer cells, wherein the population of producer cells is an enriched population that produce desired levels of the target molecule.

In another aspect, the present invention relates to a method for producing a population of engineered producer cells comprising: encapsulating each producer cell from the pool of genetically varied producer cells in a droplet to form a plurality of droplets encapsulating engineered producer cells; wherein each droplet is surrounded by an immiscible continuous phase that comprises a fluorinated-based oil or emulsion; merging each droplet containing the producer cell with a droplet encapsulating an engineered-protein based sensor cell, wherein the engineered sensor cell produces an engineered protein sensor; assaying the droplets for levels of a target molecule, wherein an engineered protein-based sensor provides a readout of the level of the desired target molecule produced by the producer cell through activation or repression of a reporter; sorting the merged droplets to isolate droplets containing producer cells that produce desired levels of the target molecule; and, recovering the cells that produce desired levels of the target molecule to form the population of producer cells, wherein the population of producer cells is an enriched population that produce desired levels of the target molecule.

In one aspect, the present invention relates to a method for producing a population of engineered producer cells comprising: encapsulating each engineered producer cell from a pool of engineered producer cells in a droplet to form a plurality of droplets encapsulating engineered producer cells, wherein the engineered producer cells comprise an engineered protein-based sensor for a desired target molecule and a reporter that is activated or repressed by the protein sensor; assaying the droplets for levels of a target molecule, wherein the engineered protein sensor provides a readout of the level of the desired target molecule produced by the engineered producer cell; isolating droplets with isolated engineered producer cells that produce desired levels of the target molecule; and, recovering the cells that produce desired levels of the target molecule to form the population of engineered producer cells, wherein the population of engineered producer cells is an enriched population of engineered producer cells that produce desired levels of the target molecule.

In another aspect, the present invention relates to method for producing a population of engineered producer cells comprising: transforming a pool of engineered producer cells with an engineered sensor plasmid, wherein the engineered sensor plasmid encodes an engineered protein sensor; encapsulating each engineered producer cell from the pool of engineered producer cells in a droplet to form a plurality of droplets encapsulating engineered producer cells; wherein the engineered producer cells comprise an engineered protein-based sensor for a desired target molecule and a reporter that is activated or repressed by the protein sensor; assaying the droplets for levels of a target molecule, wherein the engineered protein sensor provides a readout of the level of the target molecule produced by the engineered producer cell; isolating the droplets with isolated engineered producer cells that produce desired levels of the target molecule; and, recovering the cells that produce desired levels of the target molecule to form the population of engineered producer cells, wherein the population of engineered producer cells is an enriched population of engineered producer cells that produce desired levels of the target molecule.

In another aspect, the present invention relates to a method for producing a population of engineered producer cells comprising: encapsulating each engineered producer cell from a pool of engineered producer cells in a droplet to form a plurality of droplets encapsulating engineered producer cells; wherein the engineered producer cells comprise an engineered protein-based sensor for a desired target molecule and a reporter that is activated or repressed by the protein sensor; merging each droplet containing the engineered producer cell with a droplet encapsulating an engineered sensor cell, wherein the engineered sensor cell produces an engineered protein sensor; assaying the merged droplets for levels for a target molecule, wherein the engineered protein sensor provides a readout of the level of the target molecule produced by the engineered producer cell; sorting the merged droplets to isolate droplets containing engineered producer cells that produce desired levels of the target molecule; and, recovering the cells that produce desired levels of the target molecule to form the population of engineered producer cells, wherein the population of engineered producer cells is an enriched population of engineered producer cells that produce desired levels of the target molecule.

In another aspect, the present invention relates to a method for producing a population of engineered producer cells comprising: transforming a pool of engineered producer cells with an engineered sensor plasmid, wherein the engineered sensor plasmid encodes an engineered protein sensor; encapsulating each engineered producer cell from the pool of engineered producer cells in a droplet to form a plurality of droplets encapsulating engineered producer cells; wherein the engineered producer cells comprise an engineered protein-based sensor for a desired target molecule and a reporter that is activated or repressed by the protein sensor; wherein each droplet is surrounded by an immiscible continuous phase that comprises a fluorinated-based oil or emulsion; assaying the droplets for levels of a target molecule, wherein the engineered protein sensor provides a readout of the level of the target molecule produced by the engineered producer cell; isolating the droplets with isolated engineered producer cells that produce desired levels of the target molecule; recovering the cells that produce desired levels of the target molecule to form the population of engineered producer cells, wherein the population of engineered producer cells is an enriched population of engineered producer cells that produce desired levels of the target molecule.

In another aspect, the present invention relates to a method for producing a population of engineered producer cells comprising: encapsulating each engineered producer cell from the pool of engineered producer cells in a droplet to form a plurality of droplets encapsulating engineered producer cells, wherein the engineered producer cells comprise an engineered protein-based sensor for a desired target molecule and a reporter that is activated or repressed by the protein sensor; wherein each droplet is: (a) surrounded by an immiscible continuous phase that comprises a fluorinated-based oil or emulsion, and (b) comprises an engineered sensor cell, wherein the engineered sensor cell produces an engineered protein sensor; assaying the droplets for levels of a target molecule, wherein the engineered protein sensor provides a readout of the level of the target molecule produced by the engineered producer cell; isolating droplets with isolated engineered producer cells that produce desired levels of the target molecule; recovering the cells that produce desired levels of the target molecule to form the population of engineered producer cells, wherein the population of engineered producer cells is an enriched population of engineered producer cells that produce desired levels of the target molecule.

In another aspect, the present invention relates to a method for producing a population of engineered producer cells comprising: encapsulating each engineered producer cell from a pool of engineered producer cells in a droplet to form a plurality of droplets encapsulating engineered producer cells wherein the engineered producer cells comprise an engineered protein-based sensor for a desired target molecule and a reporter that is activated or repressed by the protein sensor; wherein each droplet is surrounded by an immiscible continuous phase that comprises a fluorinated-based oil or emulsion; merging each droplet containing the engineered producer cell with a droplet encapsulating an engineered sensor cell, wherein the engineered sensor cell produces an engineered protein sensor; assaying the merged droplets for levels for a target molecule, wherein the engineered protein sensor provides a readout of the level of the target molecule produced by the engineered producer cell; sorting the merged droplets to isolate droplets containing engineered producer cells that produce desired levels of the target molecule; and, recovering the cells that produce desired levels of the target molecule to form the population of engineered producer cells, wherein the population of engineered producer cells is an enriched population of engineered producer cells that produce desired levels of the target molecule.

In one aspect, the present invention relates to compositions and methods for growing and assaying clonal members of an engineered producer strain library in droplets, where each engineered producer cell also contains an engineered sensor system for reporting and assaying the production of a target molecule, where the sensor system can either reside in the genome or on a plasmid.

In another aspect, the present invention relates to the compositions and methods for growing and assaying clonal members of an engineered producer strain library in droplets in the presence of a separate engineered sensor strain (i.e., cells) that reports on the production of a target molecule by the engineered producer strain. In various embodiments, the engineered sensor strain harbors a sensor system, which is an aTF sensor which can detect the target molecule.

In yet another aspect, the present invention relates to the composition and methods for growing clonal members of an engineered producer strain library in droplets and then assaying the production levels by merging the droplet containing the engineered producer cell with a second reporting droplet containing an engineered sensor system (e.g., a cell-based sensor system or an in vitro sensor system). In various embodiments, the engineered sensor strain harbors an aTF sensor which can detect the target molecule.

In another aspect, the present technology relates to methods for growing and assaying clonal members of an engineered producer strain library in droplets, wherein each droplet is surrounded by an immiscible continuous phase that comprises a fluorinated-based oil or emulsion, and wherein each engineered producer cell additionally contains an engineered sensor plasmid for reporting and interrogating the production of a target molecule. In various embodiments, the fluorinated-based oil or emulsion is an organic oil, a fluorinated oil, a fluorinated polymer, a water-in fluorocarbon emulsion, a water-in perfluorocarbon emulsion, or combinations thereof. In various embodiments, the fluorinated-based oil or emulsion is stabilized by a particle. In some embodiments, the particle is a modified silica nanoparticle (e.g., a partially fluorinated nanoparticle, or a partially hydrophobic nanoparticle). In various embodiments, the partially fluorinated nanoparticle is a silica-based nanoparticle. In various embodiments, the particle is a partially hydrophobic silica-based nanoparticle. In various embodiments, the droplet is under microfluidic control.

In another aspect, the present technology relates to methods for growing and assaying clonal members of an engineered producer strain library in droplets in the presence of a separate engineered sensor strain that reports on the production of a target molecule by the engineered producer strain, wherein each droplet is surrounded by an immiscible continuous phase that comprises a fluorinated-based oil or emulsion, and each droplet comprises an engineered sensor cell. In various embodiments, the fluorinated-based oil or emulsion is an organic oil, a fluorinated oil, a fluorinated polymer, a water-in fluorocarbon emulsion, a water-in perfluorocarbon emulsion, or combinations thereof. In various embodiments, the fluorinated-based oil or emulsion is stabilized by a particle. In some embodiments, the particle is a modified silica nanoparticle (e.g., a partially fluorinated nanoparticle, or a partially hydrophobic nanoparticle). In various embodiments, the partially fluorinated nanoparticle is a silica-based nanoparticle. In various embodiments, the particle is a partially hydrophobic silica-based nanoparticle. In various embodiments, the droplet is under microfluidic control.

In yet another aspect, the present invention relates to methods for growing clonal members of an engineered producer strain library in droplets, wherein each droplet is surrounded by an immiscible continuous phase that comprises a fluorinated-based oil or emulsion, and then assaying the product production levels by merging the droplet containing an engineered producer cell with a second reporting droplet containing an engineered sensor system. In various embodiments, the fluorinated-based oil or emulsion is an organic oil, a fluorinated oil, a fluorinated polymer, a water-in fluorocarbon emulsion, a water-in perfluorocarbon emulsion, or combinations thereof. In various embodiments, the fluorinated-based oil or emulsion is stabilized by a particle. In some embodiments, the particle is a modified silica nanoparticle (e.g., a partially fluorinated nanoparticle, or a partially hydrophobic nanoparticle). In various embodiments, the partially fluorinated nanoparticle is a silica-based nanoparticle. In various embodiments, the particle is a partially hydrophobic silica-based nanoparticle. In various embodiments, the droplet is under microfluidic control.

Any aspect or embodiment disclosed herein can be combined with any other aspect or embodiment as disclosed herein.

### BRIEF DESCRIPTION OF THE FIGURES

FIGS. 1A-D are graphs showing TtgR sensor response variation across three MAGE-engineered E. coli MG1655 mutants in response to endogenously applied naringenin.

FIGS. 2A-D are graphs showing sensor response variation across three MAGE-engineered E. coli MG1655 mutants harboring gfp regulated by four different allosteric transcription factors (TtgR (FIG. 2A), TetR (FIG. 2B), PcaV (FIG. 2C), or QacR (FIG. 2D)) in response to their respective cognate ligand.

FIG. 3 is a graph showing interferences by diffusion across production strains. A high naringenin-producing strain (red) and low naringenin-producing strain (blue) show an averaged sensor response when cultured together (orange).

FIGS. 4A-B are graphs showing co-culture of producer cells and sensor cells as a viable strategy for screening. FIG. 4A: Sensor cells show naringenin-dependent growth and gfp production in response to naringenin produced by the co-cultured production cells (Red: non-producer+sensor cells, blue: low-producer+sensor cells, orange: high-producer+sensor cells). FIG. 4B: Sensor cells and non-producer cells (red) or high-producer cells (orange) co-cultured in droplets show easily distinguishable distributions.

FIGS. 5A-D are images showing droplet co-culture testing for naringenin production. Fluorescence microscope analysis of GFP production in co-culture with various sensor and producer cells. FIG. 5A: Sensor cells and non-producer cells. FIG. 5B: Sensor cells and low-producer cells. FIG. 5C: Sensor cells and high-producer cells. FIG. 5D: K12 sensor cells harboring a plasmid, which produces GFP in response to naringenin using a TtgR-based sensor system, encapsulated with 500 μM naringenin. Each fluorescent pixel is a bacterium within a droplet that has produced GFP in response to naringenin.

FIG. 6 is an image showing fluorescence of two sets of droplet co-incubated for 24 hours. The first set of droplets contained 500 μM naringenin and the second set of droplets contained naringenin sensor cells. If diffusion was occurring between the droplets, the sensor cells would become fluorescent over the 24 hour period.

FIG. 7 shows microscope images showing double emulsions after incubation with a mixture of either producer or non-producer cells with sensor strains.

FIGS. 8A-C are graphs showing FACS analysis of double emulsion droplets prepared with: sensor and non-producer cells (FIG. 8A); sensor and producer cells (FIG. 8B); or sensor cells with either non-producer cells or producer cells (FIG. 8C).

FIGS. 9A-C are graphs showing abrogation of diffusion in a population of growing producer cells within droplets. FIG. 9A: FACS distribution of fluorescence generated from a low naringenin producer strain when grown in droplets. FIG. 9B: FACS distribution of fluorescence generated from a high naringenin producing strain when grown in droplets. FIG. 9C: FACS distribution of fluorescence of a mixture of high a low producer strains when grown in droplets. Each droplet only contains a single producer cell at the beginning of growth and production to prevent occupancy of a single droplet by both producer strains.

FIG. 10 shows graphs that demonstrate enrichment of the high naringenin-producing strain 2E6 pNAR_{high} pSENSOR_{GFP} from the pathway negative control 2E6 pNARₙᵤₗₗ pSENSOR_{GFP}, following incubation in droplets to abrogate diffusion.

FIG. 11 is an image showing droplet encapsulation of a low- vs high-producer with the “sensor in cell”, where the same cell is responsible for both ligand and sensor production, and the fluorescent (green) read out intensity (in the high producer cell, right) is associated with the concentration of produced ligand.

FIG. 12 is an image showing a system of “co-culture sensor cells” encapsulated with either a low- or high-producer in a droplet system. Here, only non-producing cells contribute to the sensor readout, reducing burden on producing cells, where the fluorescent (green) read out intensity (in the high producer cell, left) is associated with the concentration of produced ligand.

FIG. 13 is an image showing enrichment of the high naringenin-producing strain 2E6 pNAR_{high} from the low pathway control 2E6 pNAR_{low} utilizing a droplet co-culture strategy with Δptsi::kanR pSENSOR_{GFP-Ptsi} sensor strain.

FIG. 14A, FIG. 14B, FIG. 14C, FIG. 14D, and FIG. 14E shows data of sorting doubling emulsion WOW droplets away from contaminating free E coli using FACS.

FIG. 15 shows data of making double emulsion droplets and discriminating between bright producer and dark non-producers using FACS.

FIG. 16 are images showing growth and detection of fluorescent E coli in a Pickering emulsion using a microscope.

### DETAILED DESCRIPTION

In one aspect, the present technology relates to methods for growing and assaying clonal members of an engineered producer strain library in droplets, wherein each engineered producer cell additionally contains an engineered sensor system for reporting and interrogating the production of a target molecule.

In some embodiments, engineered producer strain library is generated through a genomic diversifying technology, such as, but not limited to, Multiplexed Automated Genome Engineering (MAGE), or by plasmid-based production variation (e.g., bioprespecting of enzyme homologs, promoter variation, etc.), or by non-GMO methods, or by any other mechanism to generate production diversity. See International Patent Publication No. WO 2015/017866 and WO 2008/052101, the entire contents of which are hereby incorporated by reference.

In some embodiments, the engineered producer strain library is transformed with at least one engineered sensor plasmid or sensor system.

In some embodiments, a pool of engineered producer strains from the library are emulsified in droplets containing the growth medium and any required inducing agents including but not limited to arabinose, anhydrotetracycline, Isopropyl β-D-1-thiogalactopyranoside, heat, light, or compounds found in Table 1. In some embodiments, the emulsified strains are grown and production of the desired product occurs for a fixed period of time resulting in a build-up of product for those strains capable of producing the target molecule. In some embodiments, the fixed period of time is between about 1 to 24 hours, between about 4 to 20 hours, between about 8 to 16 hours, or between about 10 to 14 hours. In some embodiments, the fixed period of time is between about 24 to 72 hours, between about 28 to 68 hours, between about 32 to 64 hours, between about 36 to 60 hours, between about 40 to 56 hours, or between about 44 to 52 hours. In some embodiments, the fixed period of time is about 1, 2, 3, 4, 5, 6, 7, 8, 9, 10, 11, 12, 13, or 14 days. In some embodiments, the fixed period of time is about 1 week or about 2 weeks.

In some embodiments, the emulsified strains produce a response using an engineered sensor system that provides a direct readout of product levels using a reporter. In some embodiments, the direct readout of product levels is between about 1 μg/L to 100 μg/L, between about 10 μg/L to 90 μg/L, between about 20 μg/L to 80 μg/L, between about 30 μg/L to 70 μg/L, between about 40 μg/L to 60 μg/L, or between about 45 μg/L to 55 μg/L. In some embodiments, the direct readout of product levels is between about 100 μg/L to 1000 μg/L, between about 200 μg/L to 900 μg/L, between about 300 μg/L to 800 μg/L, between about 400 μg/L to 700 μg/L, or between about 500 μg/L to 600 μg/L. In some embodiments, the direct readout of product levels is between about 1 g/L to 200 g/L, between about 20 g/L to 180 g/L, between about 40 g/L to 160 g/L, between about 60 g/L to 140 g/L, between about 80 g/L to 120 g/L, or between about 90 g/L to 100 g/L. In some embodiments, the direct readout of product levels is between about 100 g/L to 500 g/L, between about 150 g/L to 450 g/L, between about 200 g/L to 400 g/L, or between about 250 g/L to 350 g/L.

By way of example, but not by way of limitation, in some embodiments, the reporter is GFP or any of the other illustrative reporter systems described below. In some embodiments, the droplets are broken, and the cells are sorted using an appropriate sorting technology like FACS.

In some embodiments, the droplets are sorted by using a dedicated droplet-sorting instrument or through forming a second bulk water emulsion and then sorting the double emulsion on a FACS. In some embodiments, the droplets are sorted according to the levels of a product produced. In some embodiments, the droplets are sorted according to a desired level of product produced by the encapsulated engineered producer cell. Once the droplets are sorted, the droplets are broken releasing the enriched engineered producer cells. In some embodiments, the genome of engineered producer cells from sorted droplets are subjected to next generation sequencing. In another embodiment, the plasmids of the producer cells are sequenced (e.g., in the case of plasmid-based pathway bioprospecting).

In some embodiments, the growth or viability of the producer strain is directly dependent and proportional to the amount of product generated. By way of a non-limiting example, in an embodiment: an engineered producer strain library is generated and transformed with the engineered sensor plasmid; the pool of transformed engineered producer strains are emulsified in droplets containing the growth medium and any required inducing agents; the transformed engineered producer cells are grown and production of product occurs for a fixed period of time resulting in a build-up of product for those cells capable of producing the target molecule; the transformed engineered producer cells respond to the build-up of product either by growing at an increased rate or by producing an agent that counteracts a toxin; the grown and viable transformed engineered producer cells are then released from the droplets forming an enriched population of engineered producer cells.

In some embodiments, engineered producer strain contains the sensor system. By way of a non-limiting example, in an embodiment: an engineered producer strain library is generated and transformed with the engineered sensor system on a plasmid; the pool of transformed engineered producer strains are emulsified in droplets containing the growth medium and any required inducing agents; the transformed engineered producer cells are grown and production of product occurs for a fixed period of time resulting in a build-up of product for those cells capable of producing the target molecule; the sensor system in the transformed engineered producer cells respond to the build-up of product through expression of a reporter, such as GFP; the engineered producer cells are then released from the droplets and sorted on a FACS.

In another aspect, the present technology relates to methods for growing and assaying clonal members of an engineered producer strain library in droplets in the presence of a separate engineered sensor strain that reports on the production of a target molecule by the engineered producer strain.

In some embodiments, an engineered production strain library is generated through a genomic diversifying technology (such as, but not limited to, CRISPR/Cas methods, MAGE, Retron-based Recombineering methods related to the SCRIBE method described by Farzadfard F, Lu TK. Genomically Encoded Analog Memory with Precise In vivo DNA Writing in Living Cell Populations. Science (New York, N.Y.). 2014; 346(6211):1256272. doi:10.1126/science.1256272), the contents of which are incorporated by reference in their entirety, or by plasmid-based production variation (e.g., bioprespecting of enzyme homologs, promoter variation, etc.), or by any other mechanism to generate production diversity, such as, e.g., non-GMO methods. By way of example, in some embodiments, non-GMO methods include, but are not limited to, chemical mutagenesis, radiation, and transposition.

In some embodiments, a pool of engineered producer strains from the library are emulsified in droplets containing growth medium, any required inducing agents, and one or more engineered sensor cells. In some embodiments, the cells are grown and production of product occurs for a fixed period of time resulting in a build-up of product for those strains capable of producing the target molecule. In some embodiments, the engineered sensor cells produce a response using an engineered sensor system that provides a direct readout of product levels using a reporter. By way of example, but not by way of limitation, in some embodiments, the reporter is GFP. In some embodiments, the droplets are sorted either through using a dedicated droplet-sorting instrument or by forming a second bulk water emulsion and then sorting the double emulsion on a FACS. Once the droplets are sorted, the droplets are broken releasing the enriched engineered producer cells. In some embodiments, the genome of engineered producer cells from sorted droplets are subjected to next generation sequencing. In another embodiment, the plasmids of the producer cells are sequenced (e.g., in the case of plasmid-based pathway bioprospecting).

In some embodiments, the growth of the engineered sensor cells in the droplet is dependent on the levels of the target molecule produced by the co-encapsulated engineered producer cells. By way of example, in some embodiments, the engineered sensor controls the expression of a key protein required for growth. This will prevent the sensor cell from utilizing production nutrients before the producer cell has time to make the target molecule.

In some embodiments, the engineered sensor cell is engineered to utilize a separate carbon source than the engineered producer cell to prevent the sensor cell from consuming the nutrients required for production.

In yet another aspect, the present invention relates to methods for growing clonal members of an engineered producer strain library in droplets and then assaying the product production levels by merging the droplet containing an engineered producer cell with a second reporting droplet containing an engineered sensor system. In some embodiments, the engineered producer strain library is generated through a genomic diversifying technology (such as, but not limited to, MAGE), or by plasmid-based production variation (e.g., bioprespecting of enzyme homologs, promoter variation, etc.), or by non-GMO methods, or by any other mechanism to generate production diversity.

In some embodiments, a pool of engineered producer strains from the library are emulsified in droplets, wherein the droplets contain growth medium and any required inducing agents. In some embodiments, the cells are grown and product production occurs for a fixed period of time, which results in a build-up of product in the engineered producer cells capable of producing the target molecule. In some embodiments, the droplets containing engineered producer cells are merged with a second set of droplets containing a sensor system (e.g., a cell-based sensor system or an in vitro sensor system) that produces a reporter.

In some embodiments, the reporter is produced proportionally to the amount of product produced by the engineered producer cells, and the merged droplets are assayed for reporter levels. In some embodiments, the merged droplets are sorted by their expression levels of the reporter. In some embodiments, the merged droplets are sorted by forming a second bulk water emulsion and then sorting the double emulsion on a FACS. In some embodiments, the merged droplets are sorted by using a dedicated droplet-sorting instrument. In some embodiments, after the droplets are sorted, the droplets are broken releasing the enriched producer cells. In some embodiments, the genome of engineered producer cells from sorted droplets are subjected to next generation sequencing. In some embodiments, the droplets are sorted according the levels of a product produced. In some embodiments, the droplets are sorted according to a desired level of product produced by the encapsulated engineered producer cell.

In some embodiments, the desired level of product produced by the encapsulated engineered producer cell is between about 1 μg/L to 100 μg/L, between about 10 μg/L to 90 μg/L, between about 20 μg/L to 80 μg/L, between about 30 μg/L to 70 μg/L, between about 40 μg/L to 60 μg/L, or between about 45 μg/L to 55 μg/L. In some embodiments, the desired level of product produced by the encapsulated engineered producer cell is between about 100 μg/L to 1000 μg/L, between about 200 μg/L to 900 μg/L, between about 300 μg/L to 800 μg/L, between about 400 μg/L to 700 μg/L, or between about 500 μg/L to 600 μg/L. In some embodiments, the desired level of product produced by the encapsulated engineered producer cell is between about 1 g/L to 200 g/L, between about 20 g/L to 180 g/L, between about 40 g/L to 160 g/L, between about 60 g/L to 140 g/L, between about 80 g/L to 120 g/L, or between about 90 g/L to 100 g/L. In some embodiments, the desired level of product produced by the encapsulated engineered producer cell is between about 100 g/L to 500 g/L, between about 150 g/L to 450 g/L, between about 200 g/L to 400 g/L, or between about 250 g/L to 350 g/L.

In some embodiments, the engineered producer cells produce an antitoxin in direct proportion to the amount of product generated and the droplet containing engineered producer cell is separately merged with a droplet having a fixed amount of toxin after the production phase. In some embodiments, the engineered producer cells that have produced a desired level of product will have produced enough antitoxin in order to survive the second emulsification. In some embodiments, after a brief incubation, the merged droplets are broken and the enriched, viable, engineered producer cell population is recovered.

In another aspect, the present technology relates to methods for growing and assaying clonal members of an engineered producer strain library in droplets, wherein each droplet is surrounded by an immiscible continuous phase that comprises an organic oil, fluorinated-based oil or emulsion, and wherein each engineered producer cell additionally contains an engineered sensor plasmid for reporting and interrogating the production of a target molecule.

In another aspect, the present technology relates to methods for growing and assaying clonal members of an engineered producer strain library in droplets in the presence of a separate engineered sensor strain that reports on the production of a target molecule by the engineered producer strain, wherein each droplet is surrounded by an immiscible continuous phase that comprises a fluorinated-based oil or emulsion, and each droplet comprises an engineered sensor cell.

In yet another aspect, the present invention relates to methods for growing clonal members of an engineered producer strain library in droplets, wherein each droplet is surrounded by an immiscible continuous phase that comprises a fluorinated-based oil or emulsion, and then assaying the product production levels by merging the droplet containing an engineered producer cell with a second reporting droplet containing an engineered sensor system.

In some embodiments of the present technology, the fluorinated-based oil or emulsion is an organic oil, a fluorinated oil, a fluorinated polymer, a water-in fluorocarbon emulsion, a water-in perfluorocarbon emulsion, or combinations thereof. In some embodiments, the fluorinated-based oil or emulsion is optionally stabilized by a particle. In some embodiments, the particle is a modified silica nanoparticle (e.g., a partially fluorinated nanoparticle, or a partially hydrophobic nanoparticle). In some embodiments, the partially fluorinated nanoparticle is a silica-based nanoparticle. In some embodiments, the particle is a partially hydrophobic silica-based nanoparticle. In some embodiments, the droplet is under microfluidic control.

In some embodiments, the emulsion is a Pickering emulsion comprising a water-immiscible liquid dispersed into aqueous phase (e.g., an oil-in-water (o/w) emulsion). In some embodiments, the emulsion comprises an organic oil. In some embodiments, the water-immiscible liquid is an oil, or an organic oil (e.g., a mineral oil, a corn oil, or a castor oil).

In some embodiments, the emulsion is a Pickering emulsion comprising aqueous droplets dispersed in a continuous oil phase (e.g., a water-in-oil (w/o) emulsion). In some embodiments, the emulsion comprises an organic oil. In some embodiments, the water-immiscible liquid is an oil, or an organic oil (e.g., a mineral oil, a corn oil, or a castor oil).

In some embodiments, the Pickering emulsion is stabilized by decreasing the chain length of the oil or organic oil. In some embodiments, the chain length of the oil or organic is decreased by at least 1 carbon atom, at least 2 carbon atoms, at least 3 carbon atoms, at least 4 carbon atoms, at least 5 carbon atoms, at least 6 carbon atoms, at least 7 carbon atoms, at least 8 carbon atoms, at least 9 carbon atoms, at least 10 carbon atoms, at least 11 carbon atoms, at least 12 carbon atoms, at least 13 carbon atoms, at least 14 carbon atoms, at least 15 carbon atoms, at least 16 carbon atoms, at least 17 carbon atoms, at least 18 carbon atoms, at least 19 carbon atoms, or at least 20 carbon atoms.

In some embodiments, the emulsion is a Pickering emulsion stabilized by a hydrocarbon. For example, the emulsion can be stabilized by hexadecane, dodecane, decane, octane, heptane, and hexane.

In some embodiments, the emulsion is a Pickering emulsion stabilized by an oil or organic oil. For example, the emulsion can be stabilized by an organic oil, such as a mineral oil, a corn oil, or a castor oil. In some embodiments, the emulsion is stabilized by an oil or organic oil combined with Tween (e.g., Tween 20, Tween 21, Tween 40, Tween 60, Tween 61, Tween 65, Tween 80, Tween 81, Tween 85), Triton X-100, Triton X-114, SPAN (e.g., SPAN 20, SPAN 40, SPAN 60, SPAN 65, SPAN 80, SPAN 85), Arlacel (e.g., Arlacel™ P135), Atlox (e.g., Atlox™ 4912), a non-ionic emulsifier, such as ABIL (e.g., ABIL® EM 90), a detergent (e.g., an ABIL-based detergent), or combinations thereof.

In some embodiments, the emulsion is a Pickering emulsion stabilized by an oil or organic oil. For example, the emulsion can be stabilized by an organic oil, such as a mineral oil, a corn oil, or a castor oil. In some embodiments, the emulsion is stabilized by an oil or organic oil combined with a protein stabilizer (e.g., bovine serum albumin (BSA), β-lactoglobulin, β-casein (BCN)). In some embodiments, the emulsion is stabilized by an oil or organic oil combined with a non-ionic detergent or sugar (e.g., glucose, fructose, lactose). In some embodiments, the protein stabilizer, non-ionic detergent or sugar reduce diffusion of organics from the second phase (e.g., an aqueous, organic, or droplet phase) into the first phase (e.g., the oil-based phase).

In some embodiments, the emulsion is a Pickering emulsion stabilized by Tween (e.g., Tween 20, Tween 21, Tween 40, Tween 60, Tween 61, Tween 65, Tween 80, Tween 81, Tween 85), Triton X-100, Triton X-114, SPAN (e.g., SPAN 20, SPAN 40, SPAN 60, SPAN 65, SPAN 80, SPAN 85), Arlacel (e.g., Arlacel™ P135), Atlox (e.g., Atlox™ 4912), a non-ionic emulsifier, such as ABIL (e.g., ABIL® EM 90), a detergent (e.g., an ABIL-based detergent), or combinations thereof.

In some embodiments, the emulsion is a Pickering emulsion stabilized a protein stabilizer (e.g., bovine serum albumin (BSA), β-lactoglobulin, β-casein (BCN)). In some embodiments, the emulsion is stabilized by a non-ionic detergent or sugar (e.g., glucose, fructose, lactose). In some embodiments, the protein stabilizer, non-ionic detergent or sugar reduce diffusion of organics from the second phase (e.g., an aqueous, organic, or droplet phase) into the first phase (e.g., the oil-based phase).

In some embodiments, the emulsion is a Pickering emulsion stabilized by a solid particle. In some embodiments, the solid particle is an inorganic or organic particle. For example, the Pickering emulsion can be stabilized by silica, calcium carbonate, clays, gold and carbon black particles, organic latex, starch, hydrogels and copolymer particles. In some embodiments, the Pickering emulsion is stabilized by proteins, bacteria and spore particles.

In some embodiments, the emulsion is a Pickering emulsion stabilized by a solid particle. In some embodiments, the particle is a modified silica nanoparticle. In some embodiments, the modified silica nanoparticle is a partially fluorinated nanoparticle. In some embodiments, the modified silica nanoparticle is a partially hydrophobic nanoparticle. In some embodiments, the partially fluorinated nanoparticle is a silica-based nanoparticle. In some embodiments, the particle is a partially hydrophobic silica-based nanoparticle. In some embodiments, the Pickering emulsion accumulates at the interface between two immiscible phases. In some embodiments, the first phase is a continuous phase and the second phase is a dispersive phase. In some embodiments, the emulsion of the present disclosure comprises a first phase that is oil-based, such as a fluorocarbon phase or an organic oil, and a second phase (e.g., an organic, aqueous, droplet, hydrocarbon, or gas phase). For example, the first phase can be a fluorocarbon phase having at least one fluorinated solvent, and the second phase can be immiscible with the fluorinated solvent, such as an organic, aqueous, droplet, hydrocarbon, or a gas phase. In some embodiments, the second phase is an aqueous phase. In some embodiments, the second phase is a hydrocarbon phase.

In some embodiments, the first phase is a fluorous phase comprising at least one fluorinated solvent, wherein the partially fluorinated nanoparticle is dispersed in the fluorinated solvent.

In some embodiments, the first phase comprises a partially hydrophobic nanoparticle dispersed in the solvent.

In some embodiments, the first phase (i.e., fluorous phase) comprises at least one fluorocarbon represented by CxFyHzXm, where X can be any element (including but not restricted to N and O), and x, y, z, and m are positive integers. In some embodiments, the first phase is a fluorous phase and comprises HFE-7500 (C₉H₅OF₁₅), HFE-7600 (C₈H₆OF₁₂), FC-40 (C₂₁F₄₈N₂), perfluorohexane (O₆F₁₄), and/or perfluoromethyldecalin (PFMD or O₁₁F₂₀) as the fluorinated solvent. The fluorinated solvent is not particularly limited, but can include a diverse range of fluorinated compounds having distinct physical properties. In some embodiments, the fluorinated solvent comprises a polar, partially fluorinated solvent with low viscosity, such as hydrofluoroethers like HFE-7500 and

HFE-7600. In some embodiments, the fluorinated solvent comprises a polar, perfluorinated solvent with high viscosity, such as FC-40. In some embodiment, the fluorinated solvent comprises a non-polar, perfluorinated solvent with low viscosity, such as C₆F₁₄. In some embodiments, the fluorinated solvent comprises a non-polar perfluorinated solvent with high viscosity, such as PFMD.

In some embodiments, the Pickering emulsion comprises a fluorocarbon phase comprising at least one fluorinated solvent, and a second phase comprising a fluid immiscible with the fluorinated solvent, wherein the partially fluorinated nanoparticle (e.g., a silica-based nanoparticle) is adsorbed to the interface of the fluorocarbon phase and the second phase.

In some embodiments, the Pickering emulsion comprises a first and a second phase comprising a fluid immiscible with the first phase, wherein the partially hydrophobic nanoparticle (e.g., a silica-based hydrophobic nanoparticle) is adsorbed to the interface of the first phase and the second phase.

In some embodiments, the Pickering emulsion comprises a continuous fluorocarbon phase, and a second phase comprising at least one aqueous, organic, hydrocarbon or gas phase droplet, or at least one gas phase bubble, dispersed in the continuous fluorocarbon phase. For example, in some embodiments, the emulsion comprises a continuous fluorocarbon phase and an aqueous phase, or the emulsion comprises a continuous fluorocarbon phase and an organic phase, or the emulsion comprises a continuous fluorocarbon phase and a hydrocarbon phase, or the emulsion comprises a continuous fluorocarbon phase and a gas phase.

In some embodiments, the Pickering emulsion comprises a continuous hydrocarbon phase, and at least one fluorocarbon phase droplet dispersed in the continuous hydrocarbon phase.

In some embodiments, the partially fluorinated nanoparticle (e.g., a silica-based nanoparticle) is adsorbed at the interface of the first phase, such as a fluorocarbon phase, and the second phase, which may be an aqueous or organic fluid, or a hydrocarbon phase.

In some embodiments, the partially hydrophobic nanoparticle (e.g., a silica-based hydrophobic nanoparticle) is adsorbed at the interface of the first phase and the second phase, which may be an aqueous or organic fluid, or droplet, or a hydrocarbon phase.

In some embodiments, the Pickering emulsion can be modified in several ways. For example, the Pickering emulsion can be modified by introducing hydrophilic polymers such as polyethylene glycol (PEG) into the dispersed phase, while F—SiO₂ nanoparticles (NPs) are pre-dispersed in the continuous phase. As drops are generated, the F—SiO₂ NPs adsorb to the water-oil interface and the hydrophilic polymers adsorb onto the surface of the F—SiO2 NPs from within the drops. For example, partially fluorinated silica nanoparticles adsorbed with PEG are referred to herein as “PEG_{ads}-F-SiO₂NPs.” In some embodiments, particles covalently grafted with hydrophilic polymers can be dispersed into the continuous phase. For example, partially fluorinated silica nanoparticles covalently grafted with PEG are referred to herein as “PEG_{covalent}-F-SiO₂NPs.” Other modifications of Pickering emulsions include, but are not limited to, covalently grafting the hydrophilic polymer onto the partially fluorinated particle (e.g., a silica-based nanoparticle). In some embodiments, the hydrophilic polymer is covalently grafted onto the partially fluorinated particle. In some embodiments, the hydrophilic polymer is not covalently linked to the partially fluorinated particle.

In some embodiments of the present disclosure, the hydrophilic polymer is a PEG. In some embodiments, the hydrophilic polymers include polyelectrolytes and non-ionic polymers such as homopolymers (e.g., polyethers, Polyacrylamide (PAM), Polyethylenimine (PEI), Poly(acrylic acid), Polymethacrylate and Other Acrylic Polymers, Poly(vinyl alcohol) (PVA), Poly(vinylpyrrolidone) (PVP)), and block co-polymers.

In some embodiments, the Pickering emulsion comprises a continuous fluorocarbon phase, and a second phase comprising an aqueous phase. In some embodiments, the aqueous phase comprises at least one hydrophilic polymer adsorbed to the partially fluorinated particle at the interface. In some embodiments, the aqueous phase droplet comprises at least one hydrophilic polymer adsorbed to the partially fluorinated nanoparticle at the interface, such as PEG_{ads}-F-SiO₂NPs or PEG_{covalent}-F-SiO2NPs.

In some embodiments, the second phase (e.g., aqueous phase) comprises about 0.01 mg/mL or more, or about 0.02 mg/mL or more, or about 0.05 mg/mL or more, or about 0.1 mg/mL or more, or about 0.2 mg/mL or more, or about 0.5 mg/mL or more, or about 1 mg/mL or more, or about 2 mg/mL or more, or about 5 mg/mL or more, or about 10 mg/mL or more of a hydrophilic polymer (e.g., PEG). In some embodiments, the aqueous phase comprises an effective amount of a hydrophilic polymer (e.g., PEG) for preventing non-specific adsorption of proteins and enzymes to the droplet interface and to maintain their activities.

In some embodiments, the fluorinated-based oil or emulsion comprises (a) a continuous fluorous phase, (b) at least one aqueous, organic, hydrocarbon or gas phase droplet, or gas bubble, dispersed in the continuous fluorous phase, and (c) at least one partially fluorinated particle (e.g., a silica-based nanoparticle) or partially hydrophobic silica nanoparticle adsorbed to the interface of the first phase (e.g., fluorous phase), and the aqueous, organic, hydrocarbon or gas phase, wherein the silica nanoparticle is partially fluorinated or partially hydrophobic.

In some embodiments, the partially fluorinated particle (e.g., a silica-based nanoparticle) is first dispersed in the fluorous phase before adsorbing to the interface of the fluorous phase and the aqueous, organic, hydrocarbon or gas phase. In some embodiments, the partially fluorinated particle is first dispersed in the aqueous, organic, hydrocarbon or gas phase before adsorbing to the interface of the fluorous phase and the aqueous or organic phase.

In some embodiments, the first phase (e.g., aqueous phase) comprises an additional component, such as buffers, salts, nutrients, therapeutic agents, drugs, hormones, antibodies, analgesics, anticoagulants, anti-inflammatory compounds, antimicrobial compositions, cytokines, growth factors, interferons, lipids, oligonucleotides polymers, polysaccharides, polypeptides, protease inhibitors, cells, nucleic acids, RNA, DNA, vasoconstrictors or vasodilators, vitamins, minerals, or stabilizers. In some embodiments, a chemical and/or biological reaction is performed in the aqueous phase.

In some embodiments, the emulsion (e.g., a Pickering emulsion) comprises a liquid phase encapsulated by a particle, such as a nanoparticle. In some embodiments, the particle is a partially fluorinated nanoparticle. In some embodiments, the partially fluorinated nanoparticle is a silica-based nanoparticle. In some embodiments, the particle is a partially hydrophobic nanoparticle. In some embodiments, the partially hydrophobic nanoparticle is a silica-based nanoparticle.

In some embodiments, the nanoparticle (e.g., silica-based nanoparticle) and combinations thereof described in the present disclosure provide stabilization against coalescence of droplets, without interfering with processes that can be carried out inside the droplets.

In some embodiments, the fluorinated-based oil or emulsion described in the present disclosure effectively prevents leakage of fluorophores and fluorogenic substrates (e.g., resorufin, fluorescein, resazurin, 4-methylumbelliferone, etc.) from the dispersed phase to the continuous phase. In some embodiments, the present disclosure effectively prevents leakage of fluorophores and fluorogenic substrates (e.g., resorufin, fluorescein, resazurin, 4-methylumbelliferone, etc.) from leakage after 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 1 day, 2 days, 3 days, 4 days or 5 days.

In some embodiments, the emulsion described herein is made by microfluidics. For example, the emulsion described herein can be made by a homogenizer or by shaking.

In some embodiments, the droplet is under microfluidic control. In some embodiments, the microfluidic control is by a microfluidic device having a microfluidic channel. In some embodiments, the nanoparticle (e.g., silica-based nanoparticle) is present in the microfluidic channel.

In some embodiments, at least about 50% (e.g., by number or weight), at least about 70%, at least about 80%, at least about 90%, or at least about 95% of the nanoparticles are partially fluorinated silica nanoparticles.

In some embodiments, the partially fluorinated silica nanoparticle comprises fluorinated groups covalently bonded on the surface of the nanoparticle. In some embodiments, the amphiphilic particle comprises fluorinated hydrocarbon groups bonded on the surface of the particle, such as fluorinated alkyl groups bonded on the surface of the particle. For example, fluorinated hydrocarbon groups include C1-020, C2-C20, C5-C20, C10-C20, C1-015, C2-C15, C5-C15, C10-C15, C1-010, C2-C10, C5-C10, and C5-C8 hydrocarbon groups, substituted with 1 or more, 2 or more, 3 or more, 4 or more, 5 or more, 6 or more, 7 or more, 8 or more, 9 or more, 10 or more, 11 or more, 12 or more, or 13 or more fluorine atoms per hydrocarbon group. Other types of halogenated hydrocarbon groups may also be bonded on the surface of the particle. In some embodiments, the amphiphilic particle is partially derivatized with at least one partially fluorinated or perfluorinated alkyl-silane. In some embodiments, the amphiphilic particle is partially derivatized with at least one partially fluorinated or perfluorinated alkyl-silane comprising a linear carbon chain. In some embodiments, the amphiphilic particle is partially derivatized with 1H, 1H, 2H, 2H-perfluorooctyltriethoxysilane (FAS) on the surface.

In some embodiments, the partially fluorinated silica nanoparticle comprises hydrophilic groups, in addition to or in place of fluorinated groups, covalently bonded on the surface of the particle. In some embodiments, the amphiphilic particle comprises amine groups covalently bonded on the surface of the particle. In some embodiments, the partially fluorinated silica nanoparticle comprises other chemical groups covalently bonded on the surface of the particle, including but not restricted to —OH, —COOH, —NH₂, —CxHy, —SO₃H, fluorophores such as fluorescein, rhodamine, macromolecules such as biotin, streptavidin, and polyethylene glycol (PEG).

In some embodiments, in addition to partially fluorinated or partially hydrophobic silica nanoparticles, other particles that have functionalizable surfaces and can be rendered amphiphilic are also compatible with embodiments of the technology disclosed herein. For example, such particles include those made from noble metals, semiconductors or organic polymers. Silica is one preferred choice because it has versatile surface functionality and is economical, biocompatible and optically inactive.

In some embodiments, an engineered producer strain library is generated through a genomic diversifying technology, such as, but not limited to, CRISPR/Cas methods, Multiplexed Automated Genome Engineering (MAGE), or by plasmid-based production variation (e.g., bioprespecting of enzyme homologs, promoter variation, etc.), or by non-GMO methods, or by any other mechanism to generate production diversity, but are not limited to, chemical mutagenesis, radiation, and transposition. See International Patent Publication No. WO 2015/017866 and WO 2008/052101, the entire contents of which are hereby incorporated by reference.

In some embodiments, the engineered producer strain library is transformed with at least one engineered sensor system, such as on a plasmid or integrated into the genome.

In some embodiments, a pool of engineered producer strains from the library are emulsified in droplets containing the growth medium and any required inducing agents including, but not limited, to arabinose, anhydrotetracycline, Isopropyl β-D-1-thiogalactopyranoside, heat, light, or compounds found in Table 1 (Target Molecule Property). In some embodiments, the emulsified strains are grown and production of the desired product occurs for a fixed period of time resulting in a build-up of product for those strains capable of producing the target molecule. In some embodiments, the fixed period of time is between about 1 to 24 hours, between about 4 to 20 hours, between about 8 to 16 hours, or between about 10 to 14 hours. In some embodiments, the fixed period of time is between about 24 to 72 hours, between about 28 to 68 hours, between about 32 to 64 hours, between about 36 to 60 hours, between about 40 to 56 hours, or between about 44 to 52 hours. In some embodiments, the fixed period of time is about 1, 2, 3, 4, 5, 6, 7, 8, 9, 10, 11, 12, 13, or 14 days. In some embodiments, the fixed period of time is about 1 week or about 2 weeks.

In some embodiments, the growth of the engineered sensor cells in the droplet is dependent on the levels of the target molecule produced by the co-encapsulated engineered producer cells. By way of example, in some embodiments, the engineered sensor controls the expression of a key protein required for growth. This will prevent the sensor cell from utilizing production nutrients before the producer cell has time to make the target molecule.

In some embodiments, the engineered sensor cell is engineered to utilize a separate carbon source than the engineered producer cell to prevent the sensor cell from consuming the nutrients required for production.

In some embodiments, the engineered sensor cells produce a response using an engineered sensor system that provides a direct readout of product levels using a reporter. By way of example, but not by way of limitation, in some embodiments, the reporter is GFP.

In some embodiments the engineered producer cell has been transformed with the sensor system that produces a reporter, either residing on a plasmid or in the genome, either before or after the producer strain library has been produced. In some embodiments, the droplets are broken after a fixe period of time as described herein, either with or without induction by some other chemical, and the producer cells are sorted on a FACS to isolate or enrich for higher producers of the desired target molecule.

In some embodiments, the droplets are sorted either through using a dedicated droplet-sorting instrument or by forming a second bulk water emulsion and then sorting the double emulsion on a FACS. In some embodiments, the droplets are sorted according to the levels of a product produced. In some embodiments, the droplets are sorted according to a desired level of product produced by the encapsulated engineered producer cell. Once the droplets are sorted, the droplets are broken releasing the enriched engineered producer cells. In some embodiments, the genome of engineered producer cells from sorted droplets are subjected to next generation sequencing. In another embodiment, the plasmids of the producer cells are sequenced (e.g., in the case of plasmid-based pathway bioprospecting).

In some embodiments, the droplets containing engineered producer cells are merged with a second set of droplets containing a sensor system (e.g., a cell-based sensor system or an in vitro sensor system) that produces a reporter.

In some embodiments, the reporter is produced proportionally to the amount of product produced by the engineered producer cells, and the merged droplets are assayed for reporter levels. In some embodiments, the merged droplets are sorted by their expression levels of the reporter. In some embodiments, the merged droplets are sorted by forming a second bulk water emulsion and then sorting the double emulsion on a FACS. In some embodiments, the merged droplets are sorted by using a dedicated droplet-sorting instrument. In some embodiments, after the droplets are sorted, the droplets are broken releasing the enriched producer cells. In some embodiments, the genome of engineered producer cells from sorted droplets are subjected to next generation sequencing. In some embodiments, the droplets are sorted according the levels of a product produced. In some embodiments, the droplets are sorted according to a desired level of product produced by the encapsulated engineered producer cell.

In some embodiments, the desired level of product produced by the encapsulated engineered producer cell is between about 1 μg/L to 100 μg/L, between about 10 μg/L to 90 μg/L, between about 20 μg/L to 80 μg/L, between about 30 μg/L to 70 μg/L, between about 40 μg/L to 60 μg/L, or between about 45 μg/L to 55 μg/L. In some embodiments, the desired level of product produced by the encapsulated engineered producer cell is between about 100 μg/L to 1000 μg/L, between about 200 μg/L to 900 μg/L, between about 300 μg/L to 800 μg/L, between about 400 μg/L to 700 μg/L, or between about 500 μg/L to 600 μg/L. In some embodiments, the desired level of product produced by the encapsulated engineered producer cell is between about 1 g/L to 200 g/L, between about 20 g/L to 180 g/L, between about 40 g/L to 160 g/L, between about 60 g/L to 140 g/L, between about 80 g/L to 120 g/L, or between about 90 g/L to 100 g/L. In some embodiments, the desired level of product produced by the encapsulated engineered producer cell is between about 100 g/L to 500 g/L, between about 150 g/L to 450 g/L, between about 200 g/L to 400 g/L, or between about 250 g/L to 350 g/L.

In some embodiments, the emulsified strains produce a response using an engineered sensor system that provides a direct readout of product levels using a reporter. In some embodiments, the direct readout of product levels is between about 1 μg/L to 100 μg/L, between about 10 μg/L to 90 μg/L, between about 20 μg/L to 80 μg/L, between about 30 μg/L to 70 μg/L, between about 40 μg/L to 60 μg/L, or between about 45 μg/L to 55 μg/L. In some embodiments, the direct readout of product levels is between about 100 μg/L to 1000 μg/L, between about 200 μg/L to 900 μg/L, between about 300 μg/L to 800 μg/L, between about 400 μg/L to 700 μg/L, or between about 500 μg/L to 600 μg/L. In some embodiments, the direct readout of product levels is between about 1 g/L to 200 g/L, between about 20 g/L to 180 g/L, between about 40 g/L to 160 g/L, between about 60 g/L to 140 g/L, between about 80 g/L to 120 g/L, or between about 90 g/L to 100 g/L. In some embodiments, the direct readout of product levels is between about 100 g/L to 500 g/L, between about 150 g/L to 450 g/L, between about 200 g/L to 400 g/L, or between about 250 g/L to 350 g/L.

By way of example, but not by way of limitation, in some embodiments, the reporter is GFP or any of the other illustrative reporter systems described below. In some embodiments, the droplets are broken, and the cells are sorted using an appropriate sorting technology like FACS.

In some embodiments, the engineered producer cells produce an antitoxin in direct proportion to the amount of product generated and the droplet containing engineered producer cell is separately merged with a droplet having a fixed amount of toxin after the production phase. In some embodiments, the engineered producer cells that have produced a desired level of product will have produced enough antitoxin in order to survive the second emulsification. In some embodiments, after a brief incubation, the merged droplets are broken and the enriched, viable, engineered producer cell population is recovered.

In some embodiments, the growth or viability of the producer strain is directly dependent and proportional to the amount of product generated. By way of a non-limiting example, in an embodiment: an engineered producer strain library is generated and transformed with the engineered sensor plasmid; the pool of transformed engineered producer strains are emulsified in droplets containing the growth medium and any required inducing agents; the transformed engineered producer cells are grown and production of product occurs for a fixed period of time resulting in a build-up of product for those cells capable of producing the target molecule; the transformed engineered producer cells respond to the build of product either by growing at an increased rate or by producing an agent that counteracts a toxin; the grown and viable transformed engineered producer cells are then released from the droplets forming an enriched population of engineered producer cells.

In another aspect, the present invention relates to a method for producing a population of engineered producer cells comprising: encapsulating each engineered producer cell from a pool of engineered producer cells in a droplet to form a plurality of droplets encapsulating engineered producer cells, wherein the engineered producer cells comprise an engineered protein-based sensor for a desired target molecule and a reporter that is activated or repressed by the protein sensor; assaying the droplets for levels of a target molecule, wherein the engineered protein sensor provides a readout of the level of the desired target molecule produced by the engineered producer cell; isolating droplets with isolated engineered producer cells that produce desired levels of the target molecule; and, recovering the cells that produce desired levels of the target molecule to form the population of engineered producer cells, wherein the population of engineered producer cells is an enriched population of engineered producer cells that produce desired levels of the target molecule.

In another aspect, the present invention relates to method for producing a population of engineered producer cells comprising: transforming a pool of engineered producer cells with an engineered sensor plasmid, wherein the engineered sensor plasmid encodes an engineered protein sensor; encapsulating each engineered producer cell from the pool of engineered producer cells in a droplet to form a plurality of droplets encapsulating engineered producer cells; wherein the engineered producer cells comprise an engineered protein-based sensor for a desired target molecule and a reporter that is activated or repressed by the protein sensor; assaying the droplets for levels of a target molecule, wherein the engineered protein sensor provides a readout of the level of the target molecule produced by the engineered producer cell; isolating the droplets with isolated engineered producer cells that produce desired levels of the target molecule; and, recovering the cells that produce desired levels of the target molecule to form the population of engineered producer cells, wherein the population of engineered producer cells is an enriched population of engineered producer cells that produce desired levels of the target molecule.

In another aspect, the present invention relates to a method for producing a population of engineered producer cells comprising: encapsulating each engineered producer cell from a pool of engineered producer cells in a droplet to form a plurality of droplets encapsulating engineered producer cells; wherein the engineered producer cells comprise an engineered protein-based sensor for a desired target molecule and a reporter that is activated or repressed by the protein sensor; merging each droplet containing the engineered producer cell with a droplet encapsulating an engineered sensor cell, wherein the engineered sensor cell produces an engineered protein sensor; assaying the merged droplets for levels for a target molecule, wherein the engineered protein sensor provides a readout of the level of the target molecule produced by the engineered producer cell; sorting the merged droplets to isolate droplets containing engineered producer cells that produce desired levels of the target molecule; and, recovering the cells that produce desired levels of the target molecule to form the population of engineered producer cells, wherein the population of engineered producer cells is an enriched population of engineered producer cells that produce desired levels of the target molecule.

In another aspect, the present invention relates to a method for producing a population of engineered producer cells comprising: transforming a pool of engineered producer cells with an engineered sensor plasmid, wherein the engineered sensor plasmid encodes an engineered protein sensor; encapsulating each engineered producer cell from the pool of engineered producer cells in a droplet to form a plurality of droplets encapsulating engineered producer cells; wherein the engineered producer cells comprise an engineered protein-based sensor for a desired target molecule and a reporter that is activated or repressed by the protein sensor; wherein each droplet is surrounded by an immiscible continuous phase that comprises a fluorinated-based oil or emulsion; assaying the droplets for levels of a target molecule, wherein the engineered protein sensor provides a readout of the level of the target molecule produced by the engineered producer cell; isolating the droplets with isolated engineered producer cells that produce desired levels of the target molecule; recovering the cells that produce desired levels of the target molecule to form the population of engineered producer cells, wherein the population of engineered producer cells is an enriched population of engineered producer cells that produce desired levels of the target molecule.

In another aspect, the present invention relates to a method for producing a population of engineered producer cells comprising: encapsulating each engineered producer cell from the pool of engineered producer cells in a droplet to form a plurality of droplets encapsulating engineered producer cells, wherein the engineered producer cells comprise an engineered protein-based sensor for a desired target molecule and a reporter that is activated or repressed by the protein sensor; wherein each droplet is: (a) surrounded by an immiscible continuous phase that comprises a fluorinated-based oil or emulsion, and (b) comprises an engineered sensor cell, wherein the engineered sensor cell produces an engineered protein sensor; assaying the droplets for levels of a target molecule, wherein the engineered protein sensor provides a readout of the level of the target molecule produced by the engineered producer cell; isolating droplets with isolated engineered producer cells that produce desired levels of the target molecule; recovering the cells that produce desired levels of the target molecule to form the population of engineered producer cells, wherein the population of engineered producer cells is an enriched population of engineered producer cells that produce desired levels of the target molecule.

In another aspect, the present invention relates to a method for producing a population of engineered producer cells comprising: encapsulating each engineered producer cell from a pool of engineered producer cells in a droplet to form a plurality of droplets encapsulating engineered producer cells wherein the engineered producer cells comprise an engineered protein-based sensor for a desired target molecule and a reporter that is activated or repressed by the protein sensor; wherein each droplet is surrounded by an immiscible continuous phase that comprises a fluorinated-based oil or emulsion; merging each droplet containing the engineered producer cell with a droplet encapsulating an engineered sensor cell, wherein the engineered sensor cell produces an engineered protein sensor; assaying the merged droplets for levels for a target molecule, wherein the engineered protein sensor provides a readout of the level of the target molecule produced by the engineered producer cell; sorting the merged droplets to isolate droplets containing engineered producer cells that produce desired levels of the target molecule; and, recovering the cells that produce desired levels of the target molecule to form the population of engineered producer cells, wherein the population of engineered producer cells is an enriched population of engineered producer cells that produce desired levels of the target molecule.

In some embodiments, the recovery comprises: (a) breaking the droplets, (b) sorting the genetically varied producer cells, and (c) growing the the producer cells on a growth medium. In some embodiments, the sorting is by fluorescence activated droplet sorting (FADS) or fluorescence activated cell sorting (FACS).

In some embodiments, the recovery comprises: (a) sorting the droplets, (b) breaking the sorted droplets, and (c) plating the broken droplets on a growth medium. In some embodiments, wherein the sorting is by fluorescence activated droplet sorting (FADS) or fluorescence activated cell sorting (FACS).

In some embodiments, breaking the droplets comprises breaking the droplets encapsulating isolated engineered producer cells that produce desired levels of the target molecule to form the population of engineered producer cells, wherein the population of engineered producer cells is an enriched population of engineered producer cells that produce desired levels of the target molecule. In some embodiments, the DNA encoding the engineered protein-based sensor is encoded episomally. In some embodiments, the DNA encoding the engineered protein-based sensor is encoded on a plasmid. In some embodiments, the DNA encoding the engineered protein-based sensor is integrated in the genome of the producer cell.

In some embodiments, the engineered protein-based sensor is or has been transfected, transduced, transformed, or otherwise made available inside the producer cells. In some embodiments, the reporter is a gene encoding a detectable marker that is activated in trans by the sensor-based protein. In some embodiments, the detectable marker is an enzyme or a selectable marker. In some embodiments, the enzyme is selected from lacZ, luciferase, or alkaline phosphatase. In some embodiments, the selectable marker is an auxotroph, antibiotic, resistance marker, a toxin, or a spectrally detectable gene product. In some embodiments, the selectable marker is a fluorescent protein. In some embodiments, the spectrally detectable gene product is detected by spectroscopy or spectrometry. In some embodiments, the gene encoding the reporter is encoded episomally. In some embodiments, the gene encoding the reporter is encoded episomally on a plasmid. In some embodiments, the gene encoding the reporter is encoded on the same plasmid as the gene encoding the engineered protein-based sensor. In some embodiments, the gene encoding the reporter is integrated in the genome.

In some embodiments, the methods further comprise producing an engineered producer strain library from which the pool of engineered producer cells is taken, wherein the engineered producer strain library is engineered to produce one or more target molecules. In some embodiments, the engineered producer strain library is produced before transforming the pool of engineered producer cells with an engineered sensor plasmid. In some embodiments, the engineered producer strain library is produced after transforming the pool of engineered producer cells with an engineered sensor plasmid.

In some embodiments, the engineered protein-based sensor and reporter are encoded within the producer cell.

In some embodiments, the engineered protein-based sensor and reporter are encoded within a co-encapsulated sensor cell. In some embodiments, the engineered protein-based sensor and reporter are encoded within a sensor cell which is encapsulated in a separate droplet, which is then merged with the droplet containing an engineered producer cell.

In one aspect, the present invention relates to a method for producing a population of engineered producer cells comprising: encapsulating each producer cell from a pool of genetically varied producer cells in a droplet to form a plurality of droplets encapsulating engineered producer cells; assaying the droplets for levels of a target molecule, wherein an engineered protein-based sensor provides a readout of the level of the desired target molecule produced by the producer cell through activation or repression of a reporter; isolating droplets with producer cells that produce desired levels of the target molecule; and, recovering the cells that produce desired levels of the target molecule to form the population of producer cells, wherein the population of producer cells is an enriched population that produce desired levels of the target molecule.

In another aspect, the present invention relates to a method for producing a population of engineered producer cells comprising: transforming a pool of engineered producer cells with an engineered sensor plasmid, wherein the engineered sensor plasmid encodes an engineered protein sensor; encapsulating each producer cell from the pool of genetically varied producer cells in a droplet to form a plurality of droplets encapsulating engineered producer cells; assaying the droplets for levels of a target molecule, wherein an engineered protein-based sensor provides a readout of the level of the target molecule produced by the producer cell through activation or repression of a reporter; isolating the droplets with producer cells that produce desired levels of the target molecule; and, recovering the cells that produce desired levels of the target molecule to form the population of producer cells, wherein the population of producer cells is an enriched population that produce desired levels of the target molecule. In another aspect, the present invention relates to a method for producing a population of engineered producer cells comprising: encapsulating each producer cell from the pool of genetically varied producer cells in a droplet to form a plurality of droplets encapsulating engineered producer cells; merging each droplet containing the producer cell with a droplet encapsulating an engineered-protein based sensor cell, wherein the engineered sensor cell produces an engineered protein sensor; assaying the droplets for levels of a target molecule, wherein an engineered protein-based sensor provides a readout of the level of the desired target molecule produced by the producer cell through activation or repression of a reporter; sorting the merged droplets to isolate droplets containing producer cells that produce desired levels of the target molecule; and, recovering the cells that produce desired levels of the target molecule to form the population of producer cells, wherein the population of producer cells is an enriched population that produce desired levels of the target molecule.

In another aspect, the present invention relates to a method for producing a population of engineered producer cells comprising: transforming a pool of engineered producer cells with an engineered sensor plasmid, wherein the engineered sensor plasmid encodes an engineered protein sensor; encapsulating each producer cell from a pool of genetically varied producer cells in a droplet to form a plurality of droplets encapsulating engineered producer cells; wherein each droplet is surrounded by an immiscible continuous phase that comprises a fluorinated-based oil or emulsion; assaying the droplets for levels of a target molecule, wherein an engineered protein-based sensor provides a readout of the level of the desired target molecule produced by the producer cell through activation or repression of a reporter; isolating droplets with producer cells that produce desired levels of the target molecule; recovering the cells that produce desired levels of the target molecule to form the population of producer cells, wherein the population of producer cells is an enriched population that produce desired levels of the target molecule.

In another aspect, the present invention relates to a method for producing a population of engineered producer cells comprising: encapsulating each producer cell from the pool of genetically varied producer cells in a droplet to form a plurality of droplets encapsulating engineered producer cells; wherein each droplet is: (a) surrounded by an immiscible continuous phase that comprises a fluorinated-based oil or emulsion, and (b) comprises an engineered sensor cell, wherein the engineered sensor cell produces an engineered protein sensor; assaying the droplets for levels of a target molecule, wherein an engineered protein-based sensor provides a readout of the level of the target molecule produced by the producer cell through activation or repression of a reporter; isolating the droplets with producer cells that produce desired levels of the target molecule; recovering the cells that produce desired levels of the target molecule to form the population of producer cells, wherein the population of producer cells is an enriched population that produce desired levels of the target molecule.

In another aspect, the present invention relates to a method for producing a population of engineered producer cells comprising: encapsulating each producer cell from the pool of genetically varied producer cells in a droplet to form a plurality of droplets encapsulating engineered producer cells; wherein each droplet is surrounded by an immiscible continuous phase that comprises a fluorinated-based oil or emulsion; merging each droplet containing the producer cell with a droplet encapsulating an engineered-protein based sensor cell, wherein the engineered sensor cell produces an engineered protein sensor; assaying the droplets for levels of a target molecule, wherein an engineered protein-based sensor provides a readout of the level of the desired target molecule produced by the producer cell through activation or repression of a reporter; sorting the merged droplets to isolate droplets containing producer cells that produce desired levels of the target molecule; and, recovering the cells that produce desired levels of the target molecule to form the population of producer cells, wherein the population of producer cells is an enriched population that produce desired levels of the target molecule.

In some embodiments, the engineered protein-based sensor and reporter are encoded within the producer cell.

In some embodiments, the engineered protein-based sensor and reporter are encoded within a co-encapsulated sensor cell. In some embodiments, the engineered protein-based sensor and reporter are encoded within a sensor cell which is encapsulated in a separate droplet, which is then merged with the droplet containing an engineered producer cell.

Engineered Sensor Strains/Cells

The engineered sensor strains (or cells) described above refer to strains or cells (e.g., bacterial, yeast, algal, plant, insect, or mammalian (human or non-human) strains or cells) that have been transformed to express at least one engineered protein sensor. As used herein, an “engineered protein sensor” refers to an allosteric protein (e.g., a sensor) that binds to and allows for the detection of a target, wherein the allosteric protein is modified. In some embodiments, the allosteric protein is modified by one or more mutations. In some embodiments, the engineered protein sensor is a non-transcription factor (non-TF) sensor.

In some embodiments, the strains (or cells) are transformed by a plasmid encoding an engineered protein sensor (e.g., an engineered sensor plasmid).

In some embodiments, the engineered protein sensor is a transcription factor. In some embodiments, the transcription factor is an allosteric transcription factor (aTF).

In some embodiments, the engineered protein sensor allows for the detection of target molecules either cellularly or acellularly.

Method of designing and making engineered protein sensors are described in PCT Application Nos.: PCT/US2017/047009 and PCT/US2017/047012 and International Patent Publication Nos.: WO 2015/127242 and WO/2016/168182, the contents of which are incorporated by reference in their entirety.

In some embodiments, the engineered protein sensor is an aTF, for instance a eukaryotic aTF.

In some embodiments, the engineered protein sensor is an engineered version of a prokaryotic transcriptional regulator family, such as, for example, a member of the LysR, AraC/XylS, TetR, LuxR, LacI, ArsR, MerR, AsnC, MarR, NtrC (EBP), OmpR, DeoR, Cold shock, GntR, and Crp families.

In some embodiments, engineered protein sensor is an engineered version of a prokaryotic transcriptional regulator family such as, for example, a member of the AbrB, AlpA, AraC, ArgR, ArsR, AsnC, BetR, BhI, CitT, CodY, ComK, CrI, Crp, CsoR, CtsR, DeoR, DnaA, DtxR, Ecf, FaeA, Fe_dep_repress, FeoC, Fis, FlhC, FlhD, Fur, GntR, GutM, Hns, HrcA, HxlR, IclR, KorB, LacI, LexA, Lsr2, LuxR, LysR, LytTR, MarR, MerR, MetJ, Mga, Mor, MtlR, NarL, NtrC, OmpR, PadR, Prd, PrrA, PucR, PuR, Rok, Ros_MucR, RpiR, RpoD, RpoN, Rrf2, RtcR, Sarp, SfsA, SinR, SorC, Spo0A, TetR, TrmB, TrpR, WhiB, Xre, YcbB, and YesN families.

In various embodiments, engineered protein sensor is an engineered version of a member of the TetR family of receptors, such as, for example, AcrR, ActlI, AmeR AmrR, ArpR, BpeR, EnvR E, EthR, HydR, IfeR, LanK, LfrR, LmrA, MtrR, Pip, PqrA, QacR, RifQ, RmrR, SimReg, SmeT, SrpR, TcmR, TetR, TtgR, TtgW, UrdK, VarR, YdeS, ArpA, Aur1B, BarA, CalR1, CprB, FarA, JadR, JadR2, MphB, NonG, PhlF, TylQ, VanT, TarA, TylP, BM1P1, Bm1P1, Bm3R1, ButR, CampR, CamR, CymR, DhaR, KstR, LexA-like, AcnR, PaaR, PsbI, ThlR, UidR, YDH1, BetI, McbR, MphR, PhaD, Q9ZF45, TtK, Yhgd or YixD, CasR, IcaR, LitR, LuxR, LuxT, OpaR, Orf2, SmcR, HapR, Ef0113, HlyllR, BarB, ScbR, MmfR, AmtR, PsrA, and YjdC.

In some embodiments, the engineered protein sensor is an engineered version of a two-component or hybrid two-component system that directly bind both a ligand and DNA or work through a protein cascade.

In some embodiments, the engineered protein sensor is a eukaryotic aTF. In some embodiments, the engineered protein sensor is an engineered version of RovM (Yersinia pseudotuberculosis), HcaR (Acinetobacter), BIcR (Agrobacterium tumefaciens), HetR (Anabaena spp.), HetR (Anabaena spp.), DesR (B. subtilis), HylllR (Bacillus cereus), PlcR (Bacillus cereus), CcpA (Bacillus megaterium), YvoA (Bacillus subtilis), AhrR (Bacillus subtilis), MntR (Bacillus subtilis), GabR (Bacillus subtilis), SinR (Bacillus subtilis), CggR (Bacillus subtilis), FapR (Bacillus subtilis), OhrR (Bacillus subtilis), PurR (Bacillus subtilis), Rrf2 (Bacillus subtilis), BmrR (Bacillus subtilis), CcpN repressor (Bacillus subtilis), TreR (Bacillus subtilis), CodY (Bacillus subtilis), yfiR (Bacillus subtilis), OhrR (Bacillus subtilis), Rex (Bacillus subtilis, Thermus thermophilus, Thermus aquaticus), NprR (Bacillus thuringiensis), BtAraR (Bacteroides thetaiotaomicron), AraR (Bacteroides thetaiotaomicron VPI), DntR (Burkholderia cepacia), CmeR (Camplylobacter jejuni), CviR (Chromobacterium violaceum), TsaR (Comamonas testosteroni), CGL2612 (Corynebacterium glatamicum), ClgR (Corynebacterium glutamicum), LldR (CGL2915) (Corynebacterium glutamicum), NtcA (Cyanobacterium Anabaena), HucR (Deinococcus radiodurans), LacI (E. coli), PrgX (Enterococcus faecalis), NikR (Helobacter pylon), LmrR (Lactococcus lactis), CcpA (Lactococcus lactis), MtbCRP (Mycobacterium tuberculosis), EthR (Mycobacterium tuberculosis), MosR (Mycobacterium tuberculosis), PhoP (Mycobacterium tuberculosis), Rv1846c (Mycobacterium tuberculosis), EthR (Mycobacterium tuberculosis), LysR (Neisseria meningitdis), NMB0573/AsnC (Neisseria meningitidis), TetR-class H (Pasteurella multocida), MexR (Pseudomonas aeruginosa), DNR (Pseudomonas aeruginosa), PA01 (Pseudomonas aeruginosa), PA2196 (Pseudomonas aeruginosa), ttgR (Pseudomonas putida), Cra (Pseudomonas putida), QscR (Psudemonas aeruginosa), ActR (S. coelicolor), SC00520 (S. coelicolor), CprB (S. coelicolor), SlyA (Salmonella enterica SlyA), FapR (Staphylococcus aureus), QacR (Staphylococcus aureus), SarZ (Staphylococcus aureus), IcaR (Staphylococcus aureus), LcaR (Staphylococcus epidermidis), SMET (Stenotrophomonas maltophilia), PcaV (SC06704) (Streptomyces coelicolor), SC04008 (Streptomyces coelicolor), NdgR (Streptomyces coelicolor), CprB (Streptomyces coelicolor), SC00253 (Streptomyces coelicolor), TetR family (Streptomyces coelicolor), SC00520 (Streptomyces coelicolor), SC04942 (Streptomyces coelicolor), SC04313 (Streptomyces coelicolor), TetR family (Streptomyces coelicolor), SC07222 (Streptomyces coelicolor), SC03205 (Streptomyces coelicolor), SC03201 (Streptomyces coelicolor), ST1710 (Sulfolobus tokodaii ST1710), HrcA (Thermotoga maritima), TM1030 (Thermotoga maritima), tm1171 (thermotoga maritime), IclR (thermotoga maritime), CarH (Thermus thermophilus), FadR (Vibrio cholerae), SmcR (Vibrio vulnificus), and RovA (Yersinia pestis).

In some embodiments, the engineered protein sensor is an engineered version of MphR, AlkS, AlkR, CdaR, BenM, RUNX1, MarR, AphA, Pex, CatM, AtzR, CatR, ClcR, CbbR, CysB, CbnR, OxyR, OccR, and CrgA.

In some embodiments, engineered protein sensor is an engineered version of aN E. coli TF, such as, for example, ArcA, AtoC, BaeR, BasR, CitB, CpxR, CreB, CusR, DcuR, DpiA, EvgA, KdpE, NarL, NarP, OmpR, PhoB, PhoP, QseB, RcsB, RstA, TorR, UhpA, UvrY, YedW, YehT, YfhK, YgiX, YpdB, ZraR, RssB, AgaR, AllR (ybbU), ArsR, AscG, BetI, BglJ, CadC, CaiF, CelD, CueR, CynR, ExuR, FecR, FucR, Fur, GatR, GutM, GutR (SrlR), ModE, MtlR, NagC, NanR (yhcK), NhaR, PhnF, PutA, RbsR, RhaR, RhaS, RpiR (AlsR), SdiA, UidR, XapR, XylR, ZntR, AllS (ybbS), Arac, ArgR, AsnC, CysB, CytR, DsdC, GalR, GalS, GcvA, GcvR, GlcC, GlpR, GntR, IdnR, LctR, Lrp, LysR, MelR, MhpR, TdcA, TdcR, TetR, TreR, TrpR, and TyrR.

In various embodiments, the engineered protein sensor is an engineered version of a plant transcriptional regulator family, such as, for example, a member of the AP2, C2H2, Dof, LATA, HD-ZIP, M-type, NF-YA, S1Fa-like, TCP, YABBY, ARF, C3H, E2F/DP, GRAS, HRT-like, MIKC, NF-YB, SAP, Trihelix, ZF-HD, ARR-B, CAMTA, EIL, GRF, HSF, MYB, NF-YC, SBP, VOZ, bHLH, B3, CO-like, ERF, GeBP, LBD, MYB_related, NZZ/SPL, SRS, WOX, bZIP, BBR-BPC, CPP, FAR1, HB-PHD, LFY, NAC, Nin-like, STAT, WRKY, BES1, DBB, G2-like, HB-other, LSD, NF-X1, RAV, TALE, and Whirly families.

In some embodiments, the engineered protein sensor is an engineered version of a yeast TF, such as, e.g., Abf1p, Abf2p, Aca1p, Ace2p, Adr1p, Aft1p, Aft2p, Arg80p, Arg81p, Aro80p, Arr1p, Asg1p, Ash1p, Azf1p, Bas1p, Cad1p, Cat8p, Cbf1p, Cep3p, Cha4p, Cin5p, Crz1p, Cst6p, Cup2p, Cup9p, Dal80p, Dal81p, Dal82p, Dot6p, Ecm22p, Ecm23p, Eds1p, Ert1p, Fhl1p, Fkh1p, Fkh2p, Flo8p, Fzf1p, Gal4p, Gat1p, Gat3p, Gat4p, Gcn4p, Gcr1p, Gis1p, Gln3p, Gsm1p, Gzf3p, Haa1p, Hac1p, Hal9p, Hap1p, Hap2p, Hap3p, Hap4p, Hap5p, Hcm1p, Hmlalpha2p, Hmra2p, Hsf1p, Ime1p, Ino2p, Ino4p, Ixr1p, Kar4p, Leu3p, Lys14p, Mac1p, Mal63p, Matalpha2p, Mbp1p, Mcm1p, Met31p, Met32p, Met4p, Mga1p, Mig1p, Mig2p, Mig3p, Mot2p, Mot3p, Msn1p, Msn2p, Msn4p, Mss11p, Ndt80p, Nhp10p, Nhp6ap, Nhp6 bp, Nrg1p, Nrg2p, Oaf1p, Pdr1p, Pdr3p, Pdr8p, Phd1p, Pho2p, Pho4p, Pip2p, Ppr1p, Put3p, Rap1p, Rdr1p, Rds1p, Rds2p, Reb1p, Rei1p, Rfx1p, Rgm1p, Rgt1p, Rim101p, Rlm1p, Rme1p, Rox1p, Rph1p, Rpn4p, Rsc30p, Rsc3p, Rsf2p, Rtg1p, Rtg3p, Sfl1p, Sfp1p, Sip4p, Skn7p, Sko1p, Smp1p, Sok2p, Spt15p, Srd1p, Stb3p, Stb4p, Stb5p, Ste12p, Stp1p, Stp2p, Stp3p, Stp4p, Sum1p, Sut1p, Sut2p, Swi4p, Swi5p, Tbf1p, Tbs1p, Tea1p, Tec1p, Tod6p, Tos8p, Tye7p, Uga3p, Ume6p, Upc2p, Urc2p, Usv1p, Vhr1p, War1p, Xbp1p, YER064C, YER130C, YER184C, YGRO67C, YKL222C, YLL054C, YLR278C, YML081W, YNR063W, YPRO13C, YPRO15C, YPRO22C, YPR196W, Yap1p, Yap3p, Yap5p, Yap6p, Yap7p, Yox1p, Yrm1p, Yrr1p, and Zap1p.

In some embodiments, the engineered protein sensor is an engineered version of a nematode TF, such as, e.g., ada-2, aha-1, ahr-1, alr-1, ast-1, atf-2, atf-5, atf-6, atf-7, athp-1, blmp-1, bra-2, brc-1, cbp-1, ccr-4, cdk-9, ced-6, ceh-1, ceh-10, ceh-12, ceh-13, ceh-14, ceh-16, ceh-17, ceh-18, ceh-19, ceh-2, ceh-20, ceh-21, ceh-22, ceh-23, ceh-24, ceh-26, ceh-27, ceh-28, ceh-30, ceh-31, ceh-32, ceh-33, ceh-34, ceh-36, ceh-37, ceh-38, ceh-39, ceh-40, ceh-41, ceh-43, ceh-44, ceh-45, ceh-48, ceh-49, ceh-5, ceh-6, ceh-60, ceh-7, ceh-8, ceh-9, cep-1, ces-1, ces-2, cey-1, cey-2, cey-3, cey-4, cfi-1, chd-3, cky-1, cnd-1, cog-1, crh-1, daf-12, daf-14, daf-16, daf-19, daf-3, daf-8, dcp-66, die-1, dlx-1, dmd-3, dmd-4, dmd-5, dmd-6, dnj-11, dpi-1, dpr-1, dpy-20, dpy-22, dpy-26, dro-1, dsc-1, efl-1, ef1-2, egl-13, egl-18, eg1-27, eg1-38, eg1-43, eg1-44, eg1-46, eg1-5, ek1-2, ek1-4, elc-1, elt-1, elt-2, elt-3, elt-4, elt-6, elt-7, end-1, end-3, eor-1, ets-4, ets-5, eya-1, fax-1, fkh-10, fkh-2, fkh-3, fkh-4, fkh-5, fkh-6, fkh-7, fkh-8, fkh-9, flt-1, fos-1, fozi-1, gei-11, gei-13, gei-3, gei-8, gfl-1, gla-3, ham-2, hbl-1, hif-1, hlh-1, hlh-10, hlh-11, hlh-12, hlh-13, hlh-14, hlh-15, hlh-16, hlh-17, hlh-19, hlh-2, hlh-25, hlh-26, hlh-27, hlh-28, hlh-29, hlh-3, hlh-30, hlh-4, hlh-6, hlh-8, hmg-1.1, hmg-1.2, hmg-1.2, hmg-11, hmg-12, hmg-3, hmg-4, hmg-5, hnd-1, hsf-1, irx-1, lag-1, let-381, let-418, lfi-1, lim-4, lim-6, lim-7, lin-1, lin-11, lin-22, lin-26, lin-28, lin-31, lin-32, lin-35, lin-39, lin-40, lin-41, lin-48, lin-49, lin-54, lin-59, lin-61, hr-1, lpd-2, lsl-1, Iss-4, Ist-3, mab-23, mab-3, mab-5, mab-9, mbf-1, mbr-1, mbr-1, mdl-1, mec-3, med-1, med-2, mef-2, mes-2, mes-4, mes-6, mex-1, mex-5, mex-6, mgl-2, mls-1, mis-2, mml-1, mua-1, mxl-1, mxl-2, mxl-3, nfi-1, ngn-1, nhr-1, nhr-10, nhr-100, nhr-101, nhr-102, nhr-103, nhr-104, nhr-105, nhr-106, nhr-107, nhr-108, nhr-109, nhr-11, nhr-110, nhr-111, nhr-112, nhr-113, nhr-114, nhr-115, nhr-116, nhr-117, nhr-118, nhr-119, nhr-12, nhr-120, nhr-121, nhr-122, nhr-123, nhr-124, nhr-125, nhr-126, nhr-127, nhr-128, nhr-129, nhr-13, nhr-130, nhr-131, nhr-132, nhr-133, nhr-134, nhr-135, nhr-136, nhr-137, nhr-138, nhr-139, nhr-14, nhr-140, nhr-141, nhr-142, nhr-143, nhr-145, nhr-146, nhr-147, nhr-148, nhr-149, nhr-15, nhr-150, nhr-152, nhr-153, nhr-154, nhr-155, nhr-156, nhr-157, nhr-158, nhr-159, nhr-16, nhr-161, nhr-162, nhr-163, nhr-164, nhr-165, nhr-166, nhr-167, nhr-168, nhr-169, nhr-17, nhr-170, nhr-171, nhr-172, nhr-173, nhr-174, nhr-175, nhr-176, nhr-177, nhr-178, nhr-179, nhr-18, nhr-180, nhr-181, nhr-182, nhr-183, nhr-184, nhr-185, nhr-186, nhr-187, nhr-188, nhr-189, nhr-19, nhr-190, nhr-191, nhr-192, nhr-193, nhr-194, nhr-195, nhr-196, nhr-197, nhr-198, nhr-199, nhr-2, nhr-20, nhr-201, nhr-202, nhr-203, nhr-204, nhr-205, nhr-206, nhr-207, nhr-208, nhr-209, nhr-21, nhr-210, nhr-211, nhr-212, nhr-213, nhr-214, nhr-215, nhr-216, nhr-217, nhr-218, nhr-219, nhr-22, nhr-220, nhr-221, nhr-222, nhr-223, nhr-225, nhr-226, nhr-227, nhr-228, nhr-229, nhr-23, nhr-230, nhr-231, nhr-232, nhr-233, nhr-234, nhr-237, nhr-238, nhr-239, nhr-241, nhr-242, nhr-243, nhr-244, nhr-245, nhr-246, nhr-247, nhr-248, nhr-249, nhr-25, nhr-250, nhr-251, nhr-252, nhr-253, nhr-254, nhr-255, nhr-256, nhr-257, nhr-258, nhr-26, nhr-260, nhr-261, nhr-262, nhr-263, nhr-264, nhr-265, nhr-266, nhr-267, nhr-268, nhr-269, nhr-27, nhr-270, nhr-271, nhr-272, nhr-273, nhr-274, nhr-275, nhr-276, nhr-277, nhr-278, nhr-28, nhr-280, nhr-281, nhr-282, nhr-283, nhr-285, nhr-286, nhr-288, nhr-3, nhr-30, nhr-31, nhr-32, nhr-33, nhr-34, nhr-35, nhr-36, nhr-37, nhr-38, nhr-39, nhr-4, nhr-40, nhr-41, nhr-42, nhr-43, nhr-44, nhr-45, nhr-46, nhr-47, nhr-47, nhr-48, nhr-49, nhr-5, nhr-50, nhr-51, nhr-52, nhr-53, nhr-54, nhr-55, nhr-56, nhr-57, nhr-58, nhr-59, nhr-6, nhr-60, nhr-61, nhr-62, nhr-63, nhr-64, nhr-65, nhr-66, nhr-67, nhr-68, nhr-69, nhr-7, nhr-70, nhr-71, nhr-72, nhr-73, nhr-74, nhr-75, nhr-76, nhr-77, nhr-78, nhr-79, nhr-8, nhr-80, nhr-81, nhr-82, nhr-83, nhr-84, nhr-85, nhr-86, nhr-87, nhr-88, nhr-89, nhr-9, nhr-90, nhr-91, nhr-92, nhr-94, nhr-95, nhr-96, nhr-97, nhr-98, nhr-99, nob-1, ntl-2, ntl-3, nurf-1, odr-7, oma-1, oma-2, pag-3, pal-1, pax-1, pax-3, peb-1, pes-1, pha-1, pha-2, pha-4, php-3, pie-1, pop-1, pos-1, pqn-47, pqn-75, psa-1, rabx-5, rbr-2, ref-1, rnt-1, sbp-1, sdc-1, sdc-2, sdc-3, sea-1, sem-4, sex-1, skn-1, sknr-1, sma-2, sma-3, sma-4, smk-1, sop-2, sox-1, sox-2, sox-3, spr-1, sptf-2, sptf-3, srab-2, srt-58, srw-49, sta-1, tab-1, taf-4, taf-5, tag-153, tag-182, tag-185, tag-192, tag-295, tag-331, tag-347, tag-350, tag-68, tag-97, tbx-11, tbx-2, tbx-30, tbx-31, tbx-32, tbx-33, tbx-34, tbx-35, tbx-36, tbx-37, tbx-38, tbx-39, tbx-40, tbx-41, tbx-7, tbx-8, tbx-9, tra-1, tra-4, ttx-1, ttx-3, unc-120, unc-130, unc-3, unc-30, unc-37, unc-39, unc-4, unc-42, unc-55, unc-62, unc-86, vab-15, vab-3, vab-7, xbp-1, zag-1, zfp-1, zim-1, zip-1, zip-2, zip-3, zip-4, zip-5, and ztf-7.

In some embodiments, the engineered protein sensor is an engineered version of a archeal TF, such as, e.g., APE_0290.1, APE_0293, APE_0880 b, APE_1602a, APE_2413, APE_2505, APE_0656 a, APE_1799 a, APE_1458 a, APE_1495 a, APE_2570.1, APE_0416 b.1, APE_0883 a, APE_0535, APE_0142, APE_2021.1, APE_0060.1, APE_0197.1, APE_0778, APE_2011.1, APE_0168.1, APE_2517.1, APE_0288, APE_0002, APE_1360.1, APE_2091.1, APE_0454, APE1862.1, APE_0669.1, APE_2443.1, APE_0787.1, APE_2004.1, APE_0025.1, APE_0153.1, AF0653, AF1264, AF1270, AF1544, AF1743, AF1807, AF1853, AF2008, AF2136, AF2404, AF0529, AF0114, AF0396, AF1298, AF1564, AF1697, AF1869, AF2271, AF1404, AF1148, AF0474, AF0584, AF1723, AF1622, AF1448, AF0439, AF1493, AF0337, AF0743, AF0365, AF1591, AF0128, AF0005, AF1745, AF0569, AF2106, AF1785, AF1984, AF2395, AF2232, AF0805, AF1429, AF0111, AF1627, AF1787, AF1793, AF1977, AF2118, AF2414, AF0643, AF1022, AF1121, AF2127, AF0139, AF0363, AF0998, AF1596, AF0673, AF2227, AF1542, AF2203, AF1459, AF1968, AF1516, AF0373, AF1817, AF1299, AF0757, AF0213, AF1009, AF1232, AF0026, AF1662, AF1846, AF2143, AF0674, Cmaq_0146, Cmaq_0924, Cmaq_1273, Cmaq_1369, Cmaq_1488, Cmaq_1508, Cmaq_1561, Cmaq_1699, Cmaq_0215, Cmaq_1704, Cmaq_1956, Cmaq_0058, Cmaq_1637, Cmaq_0227, Cmaq_0287, Cmaq_1606, Cmaq_1720, Cmaq_0112, Cmaq_1149, Cmaq_1687, Cmaq_0411, Cmaq_1925, Cmaq_0078, Cmaq_0314, Cmaq_0768, Cmaq_1206, Cmaq_0480, Cmaq_0797, Cmaq_1388, Cmaq_0152, Cmaq_0601, Cmaq_1188, Mboo_0375, Mboo_0423, Mboo_0749, Mboo_1012, Mboo_1134, Mboo_1154, Mboo_1189, Mboo_1266, Mboo_1711, Mboo_1971, Mboo_0002, Mboo_0956, Mboo_1071, Mboo_1405, Mboo_1643, Mboo_0973, Mboo_1170, Mboo_0158, Mboo_0195, Mboo_0277, Mboo_1462, Mboo_1574, Mboo_1649, Mboo_2112, Mboo_0013, Mboo_0386, Mboo_0946, Mboo_0977, Mboo_1081, Mboo_2241, Mboo_0142, Mboo_0396, Mboo_0409, Mboo_0976, Mboo_2244, Mboo_0526, Mboo_0346, Mboo_1018, Mboo_0917, Mboo_0323, Mboo_0916, Mboo_1680, Mboo_1288, Mboo_2311, Mboo_2048, Mboo_1027, Mboo_2312, rrnAC0161, rrnAC0578, rrnAC0961, rrnAC3494, rrnB0118, pNG7045, pNG6160, rrnAC0867, rrnAC2723, rrnAC3399, rrnAC3447, rrnB0052, rrnAC1653, rrnAC2779, pNG7038, rrnAC1252, rrnAC3288, rrnAC3307, rrnAC0503, rrnAC1269, pNG6047, rrnAC2622, rrnAC3290, rrnAC3365, rrnAC2301, pNG6157, rrnAC2002, rrnAC1238, rrnAC3207, pNG2039, pNG7160, rrnAC2748, rrnB0134, rrnAC2283, rrnAC1714, rrnAC1715, rrnAC2338, rrnAC2339, rrnAC2900, rrnAC0341, rrnAC3191, rrnAC1825, rrnAC2037, rrnAC0496, rrnAC3074, rrnAC2669, rrnAC0019, rrnACO231, rrnAC0564, rrnAC0640, rrnAC1193, rrnAC1687, rrnAC1786, rrnAC1895, rrnAC1953, rrnAC1996, rrnAC2017, rrnAC2022, rrnAC2052, rrnAC2070, rrnAC2160, rrnAC2472, rrnAC2785, rrnAC2936, rrnAC3167, rrnAC3451, rrnAC3486, rrnAC3490, rrnB0253, rrnB0269, pNG7159, pNG7188, pNG7357, pNG6134, rrnAC0376, rrnAC1217, rrnAC1541, rrnAC1663, rrnAC3229, pNG7223, rrnAC0440, rrnAC0535, rrnAC1742, rrnAC2519, rrnAC1764, rrnAC1777, rrnAC2762, rrnAC3264, rrnAC0417, rrnAC1303, rrnB0301, pNG6155, pNG7021, pNG7343, rrnAC1964, pNG7171, rrnAC1338, pNG7344, rrnACO230, rrnAC1971, rrnB0222, rrnAC0385, rrnAC0312, pNG7133, rrnAC0006, rrnAC1805, rrnAC3501, pNG7312, rrnAC0435, rrnAC0768, rrnAC0992, rrnAC2270, rrnAC3322, rrnB0112, rrnB0157, rrnB0161, pNG6058, pNG6092, pNG5119, pNG5140, pNG4042, pNG2006, pNG1015, rrnAC0199, rrnAC0681, rrnAC1765, rrnAC1767, pNG5067, pNG7180, pNG7307, pNG7183, rrnAC3384, pNG5131, rrnAC2777, pNG5071, rrnAC1472, pNG7308, rrnAC0869, rrnB0148, rrnAC2051, rrnAC0016, rrnAC1875, pNG6072, pNG6123, rrnAC2769, rrnAC1357, rrnAC1126, rrnAC0861, rrnAC0172, rrnAC0420, rrnAC0914, rrnAC2354, rrnAC3310, rrnAC3337, pNG5013, pNG5133, rrnAC3082, rrnB0074, pNG6075, pNG5024, rrnAC0924, rrnB0235, pNG7146, VNG0462C, VNG7122, VNG7125, VNG2445C, VNG0591C, VNG1843C, VNG0320H, VNG1123Gm, VNG1237C, VNG1285G, VNG2094G, VNG1351G, VNG1377G, VNG1179C, VNG1922G, VNG1816G, VNG0134G, VNG0194H, VNG0147C, VNG6193H, VNG2163H, VNG0101G, VNG1836G, VNG0530G, VNG0536G, VNG0835G, VNG2579G, VNG6349C, VNG1394H, VNG0113H, VNG0156C, VNG0160G, VNG0826C, VNG0852C, VNG1207C, VNG1488G, VNG6065G, VNG6461G, VNG7048, VNG7161, VNG1464G, VNG1548C, VNG0247C, VNG0471C, VNG0878Gm, VNG1029C, VNG1616C, VNG2112C, VNG6009H, VNG7007, VNG0704C, VNG1405C, VNG6318G, VNG0142C, VNG6072C, VNG6454C, VNG7053, VNG7156, VNG0703H, VNG0258H, VNG0751C, VNG1426H, VNG2020C, VNG6048H, VNG6126H, VNG6239G, VNG6478H, VNG7102, VNG6027G, VNG7023, VNG1786H, VNG2629G, VNG1598a, VNG7031, VNG6037G, VNG7171, VNG7114, VNG7038, VNG2243G, VNG6140G, VNG7100, VNG6476G, VNG6438G, VNG6050G, VNG0726C, VNG1390H, VNG6351G, VNG2184G, VNG0869G, VNG0254G, VNG6389G, VNG0315G, VNG0734G, VNG0757G, VNG1451C, VNG1886C, VNG1903Cm, VNG0985H, VNG6377H, HQ2607A, HQ2612A, HQ2779A, HQ1740A, HQ1541A, HQ1491A, HQ2619A, HQ1811A, HQ3063A, HQ3354A, HQ3642A, HQ2773A, HQ1436A, HQ2221A, HQ1414A, HQ3339A, HQ2484A, HQ3265A, HQ3620A, HQ1268A, HQ1388A, HQ1866A, HQ1563A, HQ1710A, HQ1962A, HQ1084A, HQ1739A, HQ1861A, HQ1863A, HQ2750A, HQ2664A, HQ2869A, HQ3058A, HQ3361A, HQ1277A, HQ2225A, HQ1993A, HQ1937A, HQ1088A, HQ1724A, HQ1568A, HQ2167A, HQ1230A, HQ2407A, HQ3108A, HQ1973A, HQ3260A, HQ2527A, HQ3410A, HQ2369A, HQ2564A, HQ1153A, HQ1227A, HQ3654A, HQ1867A, HQ2571A, HQ1625A, HQ3408A, HQ1689A, HQ2491A, HQ2726A, HQ2987A, HQ1041A, HQ1898A, HQ1900A, HQ1118A, Hbut_1261, Hbut_0073, Hbut_0009, Hbut_0100, Hbut_0987, Hbut_1340, Hbut_0120, Hbut_0990, Hbut_0316, Hbut_0659, Hbut_0660, Hbut_0366, Hbut_0204, Hbut_1498, Hbut_1630, Hbut_1485, Hbut_1260, Hbut_0942, Hbut_0163, Hbut_0116, Hbut_0207, Hbut_1516, Hbut_0476, Hbut_1139, Hbut_0299, Hbut_0033, Hbut_0336, Hbut_1471, Hbut_1522, Hbut_0601, Hbut_0934, Hbut_0458, Hbut_0054, Hbut_1136, Hbut_0646, Hbut_0815, Igni_0122, Igni_0494, Igni_0706, Igni_1249, Igni_0226, Igni_0308, Igni_0658, Igni_0702, Igni_0486, Igni_0602, Igni_1394, Igni_0858, Igni_1361, Igni_0354, Igni_0989, Igni_1372, Igni_1124, Msed_0229, Msed_0717, Msed_1005, Msed_1190, Msed_1224, Msed_1970, Msed_2175, Msed_0166, Msed_0688, Msed_1202, Msed_1209, Msed_1765, Msed_1956, Msed_2295, Msed_0619, Msed_0621, Msed_2232, Msed_0140, Msed_2016, Msed_0767, Msed_1126, Msed_0856, Msed_0992, Msed_1773, Msed_1818, Msed_2183, Msed_1598, Msed_1725, Msed_2276, Msed_2293, Msed_1450, Msed_0265, Msed_0492, Msed_1279, Msed_1397, Msed_1563, Msed_1566, Msed_2027, Msed_0565, Msed_0868, Msed_1371, Msed_1483, Msed_1728, Msed_1351, Msed_1733, Msed_2209, Msed_2279, Msed_2233, MTH107, MTH517, MTH899, MTH1438, MTH1795, MTH163, MTH1288, MTH1349, MTH864, MTH1193, MTH254, MTH821, MTH1696, MTH739, MTH603, MTH214, MTH936, MTH659, MTH700, MTH729, MTH967, MTH1553, MTH1328, MTH1470, MTH1285, MTH1545, MTH931, MTH313, MTH1569, MTH281, MTH1488, MTH1521, MTH1627, MTH1063, MTH1787, MTH885, MTH1669, MTH1454, Msm_1107, Msm_1126, Msm_1350, Msm_1032, Msm_0213, Msm_0844, Msm_1260, Msm_0364, Msm_0218, Msm_0026, Msm_0329, Msm_0355, Msm_0453, Msm_1150, Msm_1408, Msm_0864, Msm_0413, Msm_1230, Msm_1499, Msm_1417, Msm_1250, Msm_1090, Msm_0720, Msm_0650, Msm_0424, Msm_0631, Msm_1445, Mbur_0656, Mbur_1148, Mbur_1658, Mbur_1965, Mbur_2405, Mbur_1168, Mbur_0166, Mbur_0946, Mbur_1817, Mbur_1830, Mbur_0231, Mbur_0234, Mbur_2100, Mbur_1375, Mbur_2041, Mbur_0776, Mbur_0783, Mbur_2071, Mbur_1477, Mbur_1871, Mbur_1635, Mbur_1221, Mbur_0292, Mbur_0512, Mbur_0609, Mbur_0661, Mbur_1211, Mbur_1719, Mbur_1811, Mbur_1931, Mbur_2112, Mbur_2130, Mbur_2048, Mbur_2144, Mbur_0368, Mbur_1483, Mbur_2274, Mbur_1359, Mbur_2306, Mbur_1647, Mbur_0631, Mbur_0378, Mbur_0085, Mbur_1496, Mbur_0963, Mbur_0372, Mbur_1140, Mbur_2097, Mbur_2262, Mbur_1532, Maeo_0092, Maeo_0872, Maeo_0888, Maeo_1298, Maeo_1146, Maeo_1061, Maeo_1147, Maeo_0865, Maeo_0659, Maeo_0679, Maeo_1305, Maeo_0977, Maeo_1182, Maeo_1472, Maeo_1362, Maeo_0019, Maeo_0277, Maeo_0356, Maeo_0719, Maeo_1032, Maeo_1289, Maeo_0698, Maeo_1183, Maeo_0223, Maeo_0822, Maeo_0218, Maeo_0186, Maeo_1155, Maeo_0575, Maeo_0728, Maeo_0696, Maeo_0664, MJ0432, MJ1082, MJ1325, MJ0229, MJ0361, MJ1553, MJ1563, MJ0774, MJ1398, MJ0723, MJ0151, MJ0589a, MJECL29, MJ1647, MJ1258, MJ0168, MJ0932, MJ0080, MJ0549, MJ0767, MJ1679, MJ0568, MJ1005, MJ0529, MJ0586, MJ0621, MJ1164, MJ1420, MJ1545, MJ0272, MJ0925, MJ0300, MJ1120, MJ0379, MJ0558, MJ1254, MJ0159, MJ0944, MJ0241, MJ0173, MJ0507, MJ0782, MJ0777, MJ1503, MJ1623, MmarC5_0244, MmarC5_1146, MmarC5_0136, MmarC5_1648, MmarC5_1124, MmarC5_0967, MmarC5_1647, MmarC5_0448, MmarC5_0231, MmarC5_0579, MmarC5_1252, MmarC5_1664, MmarC5_0974, MmarC5_0625, MmarC5_1666, MmarC5_0111, MmarC5_1039, MmarC5_0316, MmarC5_0131, MmarC5_1762, MmarC5_1579, MmarC5_0380, MmarC5_0898, MmarC5_0813, MmarC5_1143, MmarC5_1694, MmarC5_1294, MmarC5_1236, MmarC5_1150, MmarC5_1138, MmarC5_1543, MmarC5_0999, MmarC5_1507, MmarC5_0876, MmarC5_0202, MmarC5_1416, MmarC5_0612, MmarC5_0571, MmarC5_1100, MmarC5_1639, MmarC5_1644, MmarC5_0714, MmarC5_0484, MmarC5_0976, MmarC6_0024, MmarC6_0026, MmarC6_0104, MmarC6_0105, MmarC6_0128, MmarC6_0252, MmarC6_0566, MmarC6_0917, MmarC6_1231, MmarC6_0916, MmarC6_1531, MmarC6_0524, MmarC6_1326, MmarC6_1644, MmarC6_0165, MmarC6_0929, MmarC6_0258, MmarC6_0037, MmarC6_0055, MmarC6_1206, MmarC6_1606, MmarC6_0210, MmarC6_0325, MmarC6_0744, MmarC6_0850, MmarC6_1025, MmarC6_1226, MmarC6_1398, MmarC6_1462, MmarC6_1664, MmarC6_1175, MmarC6_0959, MmarC6_0931, MmarC6_0136, MmarC6_0425, MmarC6_0508, MmarC6_0285, MmarC6_0184, MmarC6_0443, MmarC6_0782, MmarC6_1297, MmarC6_0861, MmarC6_0696, MmarC6_1636, MmarC6_1817, MmarC6_0908, MmarC6_0913, MmarC6_0262, MmarC6_1567, MmarC6_1748, MmarC7_0274, MmarC7_0687, MmarC7_1029, MmarC7_1513, MmarC7_1661, MmarC7_1030, MmarC7_0388, MmarC7_0257, MmarC7_0592, MmarC7_1384, MmarC7_1017, MmarC7_1655, MmarC7_0306, MmarC7_0712, MmarC7_0235, MmarC7_0457, MmarC7_0521, MmarC7_0692, MmarC7_0743, MmarC7_0919, MmarC7_1096, MmarC7_1211, MmarC7_1587, MmarC7_1702, MmarC7_0987, MmarC7_1015, MmarC7_0031, MmarC7_1400, MmarC7_1790, MmarC7_1499, MmarC7_1629, MmarC7_1168, MmarC7_1727, MmarC7_0621, MmarC7_1085, MmarC7_1260, MmarC7_0085, MmarC7_0265, MmarC7_1461, MmarC7_1038, MmarC7_1033, MmarC7_0154, MmarC7_0352, MmarC7_1652, MmarC7_1455, MMP0499, MMP1442, MMP0480, MMP0752, MMP0032, MMP0460, MMP0637, MMP0033, MMP0217, MMP1137, MMP0386, MMP1347, MMP1015, MMP0719, MMP0020, MMP0631, MMP0742, MMP1467, MMP1052, MMP0097, MMP0209, MMP0568, MMP0674, MMP0678, MMP0993, MMP1210, MMP1275, MMP1447, MMP1646, MMP1499, MMP0018, MMP1712, MMP0402, MMP0787, MMP0607, MMP0168, MMP0700, MMP0465, MMP1376, MMP0086, MMP0257, MMP0840, MMP1023, MMP0791, MMP0799, MMP0041, MMP0036, MMP0907, MMP0629, MMP1100, Mevan_0753, Mevan_1029, Mevan_1232, Mevan_1560, Mevan_1502, Mevan_1030, Mevan_0459, Mevan_0343, Mevan_0658, Mevan_1373, Mevan_1201, Mevan_1594, Mevan_1567, Mevan_1203, Mevan_0375, Mevan_0778, Mevan_0320, Mevan_0525, Mevan_0587, Mevan_0758, Mevan_0808, Mevan_0951, Mevan_1109, Mevan_1444, Mevan_1514, Mevan_1517, Mevan_1014, Mevan_0136, Mevan_0295, Mevan_1389, Mevan_1479, Mevan_1173, Mevan_1578, Mevan_1653, Mevan_0686, Mevan_1098, Mevan_1270, Mevan_0270, Mevan_0282, Mevan_1620, Mevan_1668, Mevan_1038, Mevan_1044, Mevan_1050, Mevan_1056, Mevan_1033, Mevan_0014, Mevan_0425, Mevan_0095, Mlab_0303, Mlab_0817, Mlab_0821, Mlab_1236, Mlab_1381, Mlab_0824, Mlab_0002, Mlab_0494, Mlab_0162, Mlab_0744, Mlab_1629, Mlab_0854, Mlab_0909, Mlab_1549, Mlab_0037, Mlab_0071, Mlab_0160, Mlab_1173, Mlab_1603, Mlab_1630, Mlab_1666, Mlab_1628, Mlab_0070, Mlab_1522, Mlab_0331, Mlab_1259, Mlab_0324, Mlab_1366, Mlab_1576, Mlab_0353, Mlab_0010, Mlab_0295, Mlab_0588, Mlab_1668, Mlab_0447, Mlab_0440, Mlab_0197, Mlab_1697, Mlab_1694, Mlab_1710, Mlab_1511, Mlab_0458, Mlab_0497, Mlab_0762, Mlab_0988, Mlab_0826, Memar_0011, Memar_0013, Memar_1330, Memar_1512, Memar_1567, Memar_1770, Memar_2080, Memar_0129, Memar_0140, Memar_0431, Memar_1231, Memar_1756, Memar_2162, Memar_2068, Memar_1225, Memar_0002, Memar_1921, Memar_0834, Memar_2239, Memar_1448, Memar_0817, Memar_2411, Memar_2490, Memar_2264, Memar_1471, Memar_1420, Memar_0458, Memar_1291, Memar_1391, Memar_1410, Memar_1819, Memar_2218, Memar_2347, Memar_2360, Memar_2449, Memar_1304, Memar_0106, Memar_0096, Memar_0419, Memar_1120, Memar_0385, Memar_0555, Memar_1103, Memar_1319, Memar_2487, Memar_1252, Memar_1388, Memar_0473, Memar_1524, Memar_0459, Memar_0487, Memar_1209, Memar_1387, Memar_2116, MK0576, MK1025, MK0542, MK1515, MK0506, MK1677, MK1502, MK1190, MK0175, MK0800, MK0457, MK0449, MK1380, MK1430, MK0574, MK1482, MK0984, MK0337, MK1587, MK0839, MK0619, MK0858, MK0495, MK0253, Mthe_1108, Mthe_1291, Mthe_1230, Mthe_0612, Mthe_0503, Mthe_0879, Mthe_0047, Mthe_0598, Mthe_0023, Mthe_0662, Mthe_0543, Mthe_0154, Mthe_0459, Mthe_1389, Mthe_1446, Mthe_1633, Mthe_1233, Mthe_0669, Mthe_0067, Mthe_0404, Mthe_0982, Mthe_1201, Mthe_0152, Mthe_0265, Mthe_1650, Mthe_1683, Mthe_0889, MA0191, MA0342, MA0380, MA1458, MA2551, MA3784, MA3925, MA3940, MA3952, MA4076, MA4344, MA4484, MA4576, MA0207, MA0750, MA2499, MA3597, MA4479, MA2544, MA4480, MA0504, MA2921, MA0862, MA0205, MA0460, MA0622, MA0629, MA1953, MA4398, MA4560, MA0723, MA1529, MA1551, MA2421, MA1531, MA0924, MA0575, MA1588, MA0672, MA1395, MA4075, MA1763, MA2814, MA3468, MA0022, MA4338, MA2133, MA0971, MA1005, MA0067, MA1424, MA1815, MA4668, MA2914, MA3524, MA4040, MA4267, MA3984, MA0283, MA0333, MA0414, MA1339, MA3166, MA0176, MA0180, MA0743, MA1863, MA2051, MA2055, MA2206, MA2211, MA2771, MA3189, MA4167, MA1122, MA3015, MA0079, MA0989, MA4404, MA2093, MA1671, MA4106, MA4346, MA0278, MA4331, MA0179, MA2948, MA3586, MA2761, MA1487, MA1771, MA2746, MA0364, MA2951, MA0354, MA2902, MA0368, MA2764, MA2766, MA0178, MA2782, MA2493, MA0610, MA3871, MA0287, MA0359, MA1835, MA2057, MA2207, MA2212, MA3151, MA4622, MA0926, MA1664, MA4408, MA1868, Mbar_A0506, Mbar_A0581, Mbar_A0738, Mbar_A0909, Mbar_A1363, Mbar_A1705, Mbar_A1707, Mbar_A1708, Mbar_A1719, Mbar_A2323, Mbar_A2748, Mbar_A3221, Mbar_A3427, Mbar_A1541, Mbar_A1729, Mbar_A2416, Mbar_A3312, Mbar_A0803, Mbar_A3558, Mbar_A0794, Mbar_A2965, Mbar_A1070, Mbar_A1333, Mbar_A2865, Mbar_A1639, Mbar_A3371, Mbar_A0650, Mbar_A3377, Mbar_A3361, Mbar_A0654, Mbar_A3464, Mbar_A1460, Mbar_A2808, Mbar_A1584, Mbar_A2743, Mbar_A2250, Mbar_A0507, Mbar_A0992, Mbar_A1457, Mbar_A0588, Mbar_A0122, Mbar_A2068, Mbar_A0552, Mbar_A0621, Mbar_A0692, Mbar_A1033, Mbar_A2079, Mbar_A2171, Mbar_A2318, Mbar_A2819, Mbar_A2992, Mbar_A3339, Mbar_A1265, Mbar_A1377, Mbar_A1884, Mbar_A2294, Mbar_A3663, Mbar_A2575, Mbar_A2637, Mbar_A3146, Mbar_A3330, Mbar_A3493, Mbar_A2012, Mbar_A2036, Mbar_A2688, Mbar_A3560, Mbar_A1076, Mbar_A0340, Mbar_A0520, Mbar_A1497, Mbar_A3486, Mbar_A1949, Mbar_A0475, Mbar_A0579, Mbar_A1062, Mbar_A0595, Mbar_A3297, Mbar_A3442, Mbar_A3419, Mbar_A0834, Mbar_A0787, Mbar_A2740, Mbar_A1394, Mbar_A0196, Mbar_A1270, Mbar_A3331, Mbar_A3578, Mbar_A3670, Mbar_A1080, MM0272, MM0662, MM0841, MM1040, MM1257, MM1484, MM1796, MM2237, MM2242, MM2246, MM2247, MM2261, MM2525, MM2985, MM3068, MM3208, MM1882, MM1494, MM3092, MM1595, MM3173, MM0565, MM1492, MM0266, MM1080, MM1605, MM1650, MM2809, MM2861, MM2446, MM2441, MM2040, MM1728, MM1739, MM2416, MM1825, MM0666, MM0842, MM2657, MM1332, MM2573, MM1034, MM2606, MM0247, MM0444, MM0872, MM0927, MM1363, MM2394, MM2895, MM3179, MM1005, MM3233, MM1550, MM0359, MM0361, MM1586, MM1863, MM2851, MM2853, MM3117, MM0116, MM0289, MM0346, MM1903, MM3195, MM3170, MM1085, MM0386, MM2835, MM0811, MM1042, MM1027, MM2184, MM1028, MM0432, MM2546, MM1614, MM1772, MM0692, MM0146, MM0345, MM0369, MM1554, MM2854, MM1094, MM2042, MM3115, Msp_0061, Msp_0120, Msp_1519, Msp_0293, Msp_1556, Msp_0769, Msp_0168, Msp_0614, Msp_0518, Msp_0122, Msp_0383, Msp_1218, Msp_0446, Msp_0265, Msp_0608, Msp_1143, Msp_1207, Msp_0248, Msp_0512, Msp_0823, Msp_1188, Msp_0235, Msp_0194, Msp_1057, Msp_1097, Msp_0717, Msp_0971, Msp_1360, Msp_1272, Msp_1125, Msp_0149, Mhun_0040, Mhun_0316, Mhun_0873, Mhun_1073, Mhun_1644, Mhun_2448, Mhun_2633, Mhun_2472, Mhun_0365, Mhun_0919, Mhun_0576, Mhun_0165, Mhun_2458, Mhun_0842, Mhun_0941, Mhun_1324, Mhun_1346, Mhun_2089, Mhun_1313, Mhun_1731, Mhun_1706, Mhun_0152, Mhun_0501, Mhun_1037, Mhun_2548, Mhun_2928, Mhun_3036, Mhun_0241, Mhun_1541, Mhun_2190, Mhun_0646, Mhun_1347, Mhun_1533, Mhun_1553, Mhun_1866, Mhun_1954, Mhun_0253, Mhun_1259, Mhun_1451, Mhun_2502, Mhun_0684, Mhun_2259, Mhun_0763, Mhun_1327, Mhun_1530, Mhun_2935, Mhun_2804, Mhun_0568, Mhun_0593, Mhun_1236, Mhun_1656, Mhun_2481, Mhun_2797, Mhun_0497, Mhun_0575, Mhun_0588, NEQ328, NEQ229, NEQ348, NEQ288, NEQ453, NEQ143, NEQ039, NEQ276, NEQ098, NEQ541, NP1838A, NP2534A, NP3936A, NP6056A, NP2558A, NP1144A, NP0458A, NP2490A, NP2664A, NP3370A, NP0078A, NP5052A, NP4026A, NP6200A, NP0924A, NP4828A, NP2752A, NP6106A, NP2470A, NP2474A, NP0316A, NP0252A, NP5326A, NP1048A, NP2958A, NP5152A, NP4632A, NP3636A, NP3734A, NP4552A, NP5064A, NP1496A, NP4726A, NP2878A, NP0136A, NP0162A, NP0654A, NP1532A, NP1538A, NP1564A, NP2794A, NP4286A, NP4406A, NP5130A, NP5298A, NP6030A, NP6220A, NP4436A, NP1320A, NP2146A, NP3466A, NP4796A, NP5168A, NP3046A, NP2812A, NP3608A, NP2618A, NP6176A, NP3330A, NP7054A, NP2762A, NP4124A, NP3490A, NP1128A, NP1628A, NP2114A, NP0674A, NP2366A, NP3002A, NP3776A, NP4444A, NP1296A, NP1064A, NP4080A, NP4082A, NP0534A, NP2466A, NP3718A, NP5096A, NP2220A, NP5186A, NP1684A, NP2246A, NP4822A, NP4326A, NP4106A, NP2518A, NP5272A, NP6088A, NP4258A, PTO0082, PTO0457, PTO0754, PTO0795, PTO0420, PTO1287, PTO0595, PTO0891, PTO0200, PTO1201, PTO0428, PTO0376, PTO0514, PTO0375, PTO0781, PTO1148, PTO0979, PTO0276, PTO0843, PTO0557, PTO1105, PTO1211, PTO1517, PTO1052, PTO1150, PTO0114, PTO1041, PTO1176, PTO0063, PTO0799, PTO1388, PTO1389, PTO0914, PTO1110, PTO1216, PTO0675, PTO1123, PTO0506, PTO1258, PTO1372, PTO0363, PTO1340, PTO1338, PTO1067, PTO1454, PTO1523, PTO0576, PTO0198, PAE0731, PAE0738, PAE1612, PAE2042, PAE2911, PAE1948, PAE2655, PAE0385, PAE2225, PAE3116, PAE2186, PAE1103, PAE1592, PAE1848, PAE3387, PAE1507, PAE1986, PAE3469, PAE3471, PAE0659, PAE1443, PAE1484, PAE0296, PAE2022, PAE2357, PAE1544, PAE0640, PAE2309, PAE3163, PAE2449, PAE3605, PAE0783, PAE1627, PAE1638, PAE2071, PAE3208, PAE0019, PAE0813, PAE3327, PAE0146, PAE2679, PAE2684, PAE1218, PAE1760, PAE0013, PAE3437, PAE2640, PAE3378, PAE2164, PAE0171, PAE0170, PAE3329, PAE2120, PAE1645, PAE0781, PAE2282, Pars_0006, Pars_0433, Pars_0703, Pars_0836, Pars_0990, Pars_1924, Pars_2088, Pars_2298, Pars_0264, Pars_2028, Pars_0627, Pars_1855, Pars_2059, Pars_1853, Pars_0399, Pars_0425, Pars_1561, Pars_2084, Pars_0343, Pars_0668, Pars_2155, Pars_0438, Pars_1526, Pars_2364, Pars_1428, Pars_0037, Pars_1981, Pars_1988, Pars_2104, Pars_0057, Pars_0792, Pars_0504, Pars_0550, Pars_1742, Pars_1776, Pars_0311, Pars_0752, Pars_1087, Pars_1872, Pars_1005, Pars_0806, Pars_2186, Pars_2187, Pars_1743, Pars_2132, Pars_1649, Pars_1976, Pars_0035, Pars_1810, Pars_2125, Pcal_0142, Pcal_0905, Pcal_0946, Pcal_0412, Pcal_0495, Pcal_0687, Pcal_1273, Pcal_0822, Pcal_1595, Pcal_1185, Pcal_0610, Pcal_1183, Pcal_2085, Pcal_0796, Pcal_0536, Pcal_1689, Pcal_0008, Pcal_1198, Pcal_1653, Pcal_0295, Pcal_1924, Pcal_1927, Pcal_0200, Pcal_0589, Pcal_0596, Pcal_2145, Pcal_0791, Pcal_0023, Pcal_1415, Pcal_1735, Pcal_0266, Pcal_0346, Pcal_0543, Pcal_0792, Pcal_1032, Pcal_0159, Pcal_1078, Pcal_1890, Pcal_1316, Pcal_1055, Pcal_0584, Pcal_1734, Pcal_2147, Pcal_1638, Pcal_2070, Pisl_1759, Pisl_2001, Pisl_0858, Pisl_1838, Pisl_0307, Pisl_0653, Pisl_1426, Pisl_1248, Pisl_1639, Pisl_1808, Pisl_0995, Pisl_1590, Pisl_0997, Pisl_0709, Pisl_1563, Pisl_1834, Pisl_1578, Pisl_0622, Pisl_1613, Pisl_0725, Pisl_1023, Pisl_0410, Pisl_1076, Pisl_1655, Pisl_1662, Pisl_1854, Pisl_0045, Pisl_1100, Pisl_0810, Pisl_0572, Pisl_1971, Pisl_1303, Pisl_1717, Pisl_0038, Pisl_0979, Pisl_0565, Pisl_1878, Pisl_0807, Pisl_1975, Pisl_1974, Pisl_0573, Pisl_0955, Pisl_1667, Pisl_1074, Pisl_1008, Pisl_1250, PAB2298, PAB1869, PAB0625, PAB0751, PAB1002, PAB2328, PAB0125, PAB0208, PAB0619, PAB1229, PAB1227, PAB0108, PAB0322, PAB0392, PAB2312, PAB7115, PAB2062.1n, PAB1938, PAB1236, PAB2257, PAB7359, PAB2299, PAB0758a, PAB3089, PAB3117, PAB0960, PAB1522.1n, PAB2324, PAB0714, PAB2311, PAB1533, PAB0211, PAB2104, PAB2035, PAB0475, PAB0842, PAB0668, PAB7155, PAB3293, PAB0917, PAB0661, PAB0953, PAB1243, PAB1544, PAB0331, PAB1922, PAB7338, PAB0603, PAB1517, PAB1726, PAB1641, PAB1642, PAB0976, PAB1912, PAB0950, PAB0838, PF0007, PF0230, PF1072, PF1406, PF2051, PF0113, PF0232, PF1790, PF1088, PF0095, PF1734, PF0054, PF1543, PF1732, PF0250, PF0739, PF1231, PF1601, PF1022, PF1893, PF0607, PF0829, PF1722, PF1831, PF0322, PF0524, PF2053, PF0851, PF1194, PF0055, PF0505, PF0512, PF1386, PF1735, PF1794, PF1851, PF0691, PF0487, PF0988, PF1029, PF2062, PF0263, PF0709, PF1476, PF0584, PF1198, PF0535, PF1295, PF1338, PF1337, PF0687, PF1377, PF0491, PF0496, PF0661, PF1743, PF0124, PF0649, PH0062, PH1101, PH0199, PH0289, PH0825, PH1061, PH1406, PH1744, PH1930, PH1932, PH0977, PH0952, PH0180, PH1692, PH0045, PH1856.1n, PH0061, PHS045, PH1592, PH1916, PH0140, PH1519, PHS023, PH1055, PHS034, PHS051, PHS046, PH0601, PHS024, PH0468, PH1163, PH0046, PH0787, PH0783, PH1471, PH1691, PH1748, PH1808, PH0660, PH0804, PH0995, PH0614, PH0914, PH0718.1n, PH1080, PH0763, PH1009, PH1161, PH1160, PH1482, PH0864, PH0619, PH0751, PH0799, PH1034, PH0588, Smar_0567, Smar_0017, Smar_0429, Smar_1295, Smar_0048, Smar_0184, Smar_0954, Smar_1451, Smar_0205, Smar_0336, Smar_0366, Smar_1141, Smar_0476, Smar_0879, Smar_0338, Smar_0194, Smar_0612, Smar_0915, Smar_1254, Smar_1341, Smar_0279, Smar_1409, Smar_0319, Smar_0758, Smar_1442, Smar_1514, Smar_1075, Smar_1322, Smar_0054, Smar_1137, Smar_1250, Smar_0918, Smar_0086, Saci_0006, Saci_0446, Saci_1068, Saci_1787, Saci_1979, Saci_0800, Saci_1710, Saci_2236, Saci_2266, Saci_2136, Saci_0992, Saci_0731, Saci_0752, Saci_1304, Saci_1588, Saci_0944, Saci_0843, Saci_0942, Saci_0264, Saci_1391, Saci_0476, Saci_1223, Saci_0112, Saci_0048, Saci_1851, Saci_0455, Saci_2061, Saci_2116, Saci_2167, Saci_2183, Saci_2296, Saci_0655, Saci_1344, Saci_1505, Saci_2359, Saci_1192, Saci_2313, Saci_0161, Saci_0102, Saci_0133, Saci_0874, Saci_1219, Saci_1482, Saci_1670, Saci_1956, Saci_2112, Saci_0488, Saci_0483, Saci_1180, Saci_1171, Saci_1186, Saci_1242, Saci_0489, Saci_1005, Saci_2352, Saci_0380, Saci_1336, Saci_1230, Saci_2283, Saci_1107, Saci_0866, Saci_1341, Saci_0652, Saci_0842, Saci_1161, SSO00458, SSO00620, SSO09953, SSO02688, SSO00200, SSO01423, SSO02114, SSO02347, SSO03103, SSO05522, SSO00977, SSO00606, SSO02131, SSO010340, SSO00157, SSO06024, SSO00659, SSO05826, SSO010342, SSO03242, SSO00669, SSO02273, SSO02244, SSO01589, SSO01255, SSO00447, SSO00785, SSO01008, SSO01219, SSO01306, SSO01536, SSO02058, SSO03061, SSO03080, SSO01868, SSO03097, SSO02474, SSO03188, SSO00107, SSO00270, SSO00387, SSO00942, SSO01066, SSO00040, SSO01264, SSO01384, SSO01750, SSO01897, SSO02090, SSO02132, SSO02933, SSO02992, SSO02897, SSO03176, SSO00048, SSO00365, SSO01082, SSO01108, SSO01352, SSO01101, SSO01110, SSO02652, SSO01695, SSO01748, SSO02957, SSO02327, SSO00038, SSO00049, SSO00994, SSO02138, SSO02571, SSO00951, SSO02206, SSO02089, SSO02598, SSO02506, SSO00446, SSO00946, SSO00266, SSO00426, SSO02073, ST0236, ST1060, ST1064, ST1076, ST1486, ST1604, ST1889, STS229, ST0720, ST0173, STS095, ST2514, ST1022, ST2372, ST0193, ST0489, ST1115, ST1301, STSO42, ST1473, STS071, STS074, STS163, STS072, STS250, STS248, ST2039, ST2236, ST2114, ST2562, ST0051, ST0164, ST0722, ST2550, ST1593, ST0256, ST0331, ST1268, ST2084, ST2190, ST1409, ST0808, STS035, ST0758, ST1043, ST1386, ST1710, ST1716, ST1867, ST1890, ST2388, STS086, ST0749, ST0837, ST0980, ST2050, ST0757, ST0766, ST2210, ST1773, ST1340, ST1054, ST1275, ST1007, ST1041, ST0684, ST0072, ST0349, ST1271, ST0334, ST1630, ST0371, TK0063, TK0559, TK1041, TK1261, TK1826, TK1881, TK2190, TK1086, TK1883, TK1955, TK2291, TK2134, TK1285, TK1487, TK0168, TK1331, TK0567, TK0834, TK1491, TK1210, TK2110, TK2052, TK0143, TK1413, TK2289, TK2270, TK1815, TK1439, TK0695, TK1259, TK0107, TK0448, TK1057, TK1058, TK1272, TK0697, TK0126, TK0539, TK1266, TK1688, TK2197, TK2218, TK1489, TK1339, TK0142, TK0169, TK1246, TK0770, TK1494, TK1924, TK2107, TK1143, TK1654, TK0151, TK0779, TK2151, TK0132, TK2287, TK1280, TK2024, TK0471, TK1769, TK1913, TK1050, Tpen_0466, Tpen_0552, Tpen_0860, Tpen_1509, Tpen_0232, Tpen_0836, Tpen_1499, Tpen_0577, Tpen_0018, Tpen_0579, Tpen_0150, Tpen_0366, Tpen_0869, Tpen_0668, Tpen_0348, Tpen_1236, Tpen_0124, Tpen_0102, Tpen_0973, Tpen_1621, Tpen_0378, Tpen_0538, Tpen_0707, Tpen_0776, Tpen_0069, Tpen_0090, Tpen_0173, Tpen_1796, Tpen_1358, Tpen_0115, Tpen_1464, Tpen_1595, Tpen_1401, Tpen_0901, Tpen_1818, Tpen_0293, Tpen_0690, Tpen_0374, Tpen_0710, Tpen_0070, Tpen_1551, Tpen_1591, Tpen_1154, Tpen_1562, Ta0472, Ta0731, Ta1110, Ta0115, Ta1173, Ta1443, Ta0185, Ta0678, Ta0608, Ta0257, Ta0981, Ta0093, Ta0550m, Ta0842, Ta0872, Ta1362m, Ta0736, Ta1394, Ta0166, Ta0675, Ta0748, Ta1231, Ta1186, Ta0106, Ta0948, Ta1282m, Ta1363, Ta0131, Ta0320m, Ta0411, Ta1064, Ta1166, Ta1218, Ta1503, Ta0201, Ta0346, Ta1496, Ta0868m, Ta1061m, Ta0825, Ta0795, Ta0199, Ta1485, Ta0945, Ta0940, Ta0134, Ta0685, Ta0890, Ta1324, TVN0192, TVN0983, TVN1251, TVN0658, TVN0295, TVN1196, TVN1337, TVN1127, TVN0160, TVN0945, TVN0938, TVN0292, TVN0236, TVN0364, TVN0447, TVN0906, TVN1422, TVN0185, TVN0291, TVN0514, TVN1093, TVN0210, TVN1272, TVN0519, TVN0603, TVN1246, TVN1408, TVN1203, TVN1162, TVN0516, TVN1265, TVN1392, TVN1493, TVN0934, TVN0728, TVN0704, TVN1394, TVN0084, TVN1083, TVN1089, TVN0213, TVN1149, TVN0972, TVN0377, LRC567, RCIX1274, RCIX1420, RCIX1655, RCIX1698, RCIX2213, RCIX2336, RRC298, RRC486, RRC76, RCIX1140, RCIX2193, RCIX670, RCIX684, RCIX808, RCIX820, LRC582, RCIX785, LRC109, RCIX103, RCIX105, RCIX106, RCIX1508, RCIX1739, RCIX2247, RRC465, RCIX1740, RCIX2328, RRC178, LRC575, RCIX1349, RCIX1520, LRC520, RCIX125, RCIX1430, RCIX148, RCIX1527, RCIX1743, RCIX2456, RCIX449, RCIX571, RRC212, RCIX960, LRC190, RCIX1230, RCIX414, RCIX1747, LRC319, RCIX1292, RCIX1376, RCIX2173, RCIX2196, RRC154, RCIX1238, RCIX1068, RCIX1190, RCIX1914, RCIX2177, RCIX824, RCIX989, RCIX2108, LRC274, LRC304, RCIX1189, RCIX1785, RCIX1790, and RCIX90.

In various embodiments, the engineered protein sensor and/or switch is an engineered version of a B. subtilis TF, such as, e.g., Abh, AbrB, AcoR, AdaA, AhrC, AlaR, AlsR, AnsR, AraR, ArfM, ArsR, AzlB, BirA, BkdR, BltR, BmrR, CcpA, CcpB, CcpC, CggR, CheB, CheV, CheY, CitR, CitT, CodY, ComA, ComK, ComZ, CssR, CtsR, DctR, DegA, DegU, DeoR, DnaA, ExuR, FNR, FruR, Fur, GabR, GerE, GlcK, GlcR, GlcT, GlnR, GlpP, GltC, GltR, GntR, GutR, Hbs, Hpr, HrcA, HtrA, HutP, HxlR, IoiR, Ipi, KdgR, KipR, LacR, LevR, LexA, LicR, LicT, LmrA, LrpA, LrpB, LrpC, LytR, LytT, ManR, MecA, Med, MntR, MsmR, Mta, MtlR, MtrB, NhaX, PadR, PaiA, PaiB, PerR, Phage PBSX transcriptional regulator, PhoP, PksA, PucR, PurR, PyrR, RbsR, ResD, Rho, RocR, Rok, RplT, RsfA, SacT, SacV, SacY, SenS, SigA, SigB, SigD, SigE, SigF, SigG, SigH, SigI, SigK, SigL, SigM, SigV, SigW, SigX, SigY, SigZ, SinR, Slr, SplA, Spo0A, Spo0F, SpolIID, SpoVT, TenA, TenI, TnrA, TreR, TrnB-Gly1, TrnB-Phe, TrnD-Cys, TrnD-Gly, TrnD-Phe, TrnD-Ser, TrnD-Trp, TrnD-Tyr, TrnI-Gly, TrnI-Thr, TrnJ-Gly, TrnS-Leu2, TrnSL-Tyr1, TrnSL-Val2, Xpf, Xre, XylR, YacF, YazB, YbaL, YbbB, YbbH, YbdJ, YbfA, YbfI, YbfP, YbgA, YcbA, YcbB, YcbG, YcbL, YccF, YccH, YceK, YcgE, YcgK, YclA, YclJ, YcnC, YcnK, YcxD, YczG, YdcH, YdcN, YdeB, YdeC, YdeE, YdeF, YdeL, YdeP, YdeS, YdeT, YdfD, YdfF, YdfI, YdfL, YdgC, YdgG, YdgJ, YdhC, YdhQ, YdhR, YdiH, YdzF, YerO, YesN, YesS, YetL, YezC, YezE, YfhP, YfiA, YfiF, YfiK, YfiR, YfiV, YfmP, YhbI, YhcB, YhcF, YhcZ, YhdE, YhdI, YhdQ, YhgD, YhjH, YhjM, YisR, YisV, YjbD, YjdI, YkmA, YkoG, YkoM, YkvE, YkvN, YkvZ, YlaC, YlbO, YlpC, YmfC, YneI, YoaU, YobD, YobQ, YocG, YodB, YofA, YonR, YopO, YopS, YozA, YozG, YpbH, YplP, YpoP, YpuH, YqaE, YqaF, YgaG, YqfL, YqzB, YraB, YraN, YrdQ, YrhI, YrhM, YrkP, YrxA, YrzC, YsiA, YsmB, YtcD, YtdP, YtlI, YtrA, YtsA, YttP, YtzE, YufM, YulB, YurK, YusO, YusT, YuxN, YvaF, YvaN, YvaO, YvaP, YvbA, YvbU, YvcP, YvdE, YvdT, YvfI, YyfU, YvhJ, YvkB, YvmB, YvnA, YvoA, YvqC, YvrH, YvrI, YvyD, YvzC, YwaE, YwbI, YwcC, YwfK, YwgB, YwhA, YwoH, YwqM, YwrC, YwtF, YxaD, YxaF, YxbF, YxdJ, YxjL, YxjO, YyaN, YybA, YybE, YybR, YycF, YydK, and Zur.

In various embodiments, the engineered protein sensor and/or switch is an engineered version of a Arabidopsis thaliana TF, such as, e.g., AT1G01060, AT1G01380, AT1G01530, AT1G02340, AT1G04370, AT1G06160, AT1G07640, AT1G09530, AT1G09770, AT1G10170, AT1G12610, AT1G12860, AT1G12980, AT1G13960, AT1G14350, AT1G14920, AT1G15360, AT1G16490, AT1G18570, AT1G19220, AT1G19350, AT1G19850, AT1G21970, AT1G22070, AT1G23420, AT1G24260, AT1G24590, AT1G25560, AT1G26310, AT1G26870, AT1G26945, AT1G27730, AT1G28300, AT1G30210, AT1G30330, AT1G30490, AT1G32330, AT1G32540, AT1G32640, AT1G32770, AT1G33240, AT1G34370, AT1G34790, AT1G35515, AT1G42990, AT1G45249, AT1G46768, AT1G47870, AT1G51700, AT1G52150, AT1G52880, AT1G52890, AT1G53230, AT1G53910, AT1G54060, AT1G55580, AT1G55600, AT1G56010, AT1G56650, AT1G62300, AT1G62360, AT1G63650, AT1G65620, AT1G66350, AT1G66390, AT1G66600, AT1G67260, AT1G68640, AT1G69120, AT1G69180, AT1G69490, AT1G69600, AT1G70510, AT1G71030, AT1G71692, AT1G71930, AT1G73730, AT1G74930, AT1G75080, AT1G76420, AT1G77850, AT1G78600, AT1G79180, AT1G79580, AT1G79840, AT2G01500, AT2G01570, AT2G01930, AT2G02450, AT2G03340, AT2G16910, AT2G17950, AT2G20180, AT2G22300, AT2G22540, AT2G22630, AT2G22770, AT2G23760, AT2G24570, AT2G26150, AT2G27050, AT2G27300, AT2G27990, AT2G28160, AT2G28350, AT2G28550, AT2G28610, AT2G30250, AT2G30432, AT2G33810, AT2G33835, AT2G33860, AT2G33880, AT2G34710, AT2G36010, AT2G36270, AT2G36890, AT2G37260, AT2G37630, AT2G38470, AT2G40220, AT2G40950, AT2G42200, AT2G42830, AT2G43010, AT2G45190, AT2G45660, AT2G46270, AT2G46410, AT2G46680, AT2G46770, AT2G46830, AT2G46870, AT2G46970, AT2G47190, AT2G47460, AT3G01140, AT3G01470, AT3G02990, AT3G03450, AT3G04670, AT3G07650, AT3G10800, AT3G11440, AT3G12250, AT3G13540, AT3G13890, AT3G15170, AT3G15210, AT3G15500, AT3G15510, AT3G16770, AT3G16857, AT3G17609, AT3G18990, AT3G19290, AT3G20310, AT3G20770, AT3G22170, AT3G23130, AT3G23250, AT3G24650, AT3G25710, AT3G26744, AT3G26790, AT3G27785, AT3G27810, AT3G27920, AT3G28470, AT3G28910, AT3G44750, AT3G46640, AT3G48160, AT3G48430, AT3G49940, AT3G50410, AT3G51060, AT3G54220, AT3G54320, AT3G54340, AT3G54620, AT3G55370, AT3G56400, AT3G58070, AT3G58780, AT3G59060, AT3G61850, AT3G61890, AT3G61910, AT3G62420, AT4G00120, AT4G00180, AT4G00220, AT4G01250, AT4G01540, AT4G02560, AT4G04450, AT4G08150, AT4G09820, AT4G09960, AT4G15090, AT4G16110, AT4G16780, AT4G17750, AT4G18960, AT4G20380, AT4G21330, AT4G21750, AT4G23550, AT4G23810, AT4G24020, AT4G24240, AT4G24470, AT4G24540, AT4G25470, AT4G25480, AT4G25490, AT4G25530, AT4G26150, AT4G27330, AT4G27410, AT4G28110, AT4G28610, AT4G30080, AT4G31550, AT4G31800, AT4G31920, AT4G32730, AT4G32880, AT4G32980, AT4G34000, AT4G34590, AT4G34990, AT4G35900, AT4G36730, AT4G36870, AT4G36920, AT4G36930, AT4G37540, AT4G37650, AT4G37750, AT4G38620, AT5G01900, AT5G02030, AT5G02470, AT5G03150, AT5G03680, AT5G03790, AT5G04240, AT5G05410, AT5G06070, AT5G06100, AT5G06650, AT5G06950, AT5G06960, AT5G07100, AT5G07690, AT5G07700, AT5G08130, AT5G09750, AT5G10140, AT5G10510, AT5G11260, AT5G11510, AT5G12870, AT5G13790, AT5G14010, AT5G14750, AT5G14960, AT5G15840, AT5G15850, AT5G16560, AT5G16820, AT5G17300, AT5G17430, AT5G18560, AT5G18830, AT5G20240, AT5G20730, AT5G21120, AT5G22220, AT5G22570, AT5G23000, AT5G23260, AT5G26660, AT5G35550, AT5G35770, AT5G37020, AT5G37260, AT5G40330, AT5G40350, AT5G40360, AT5G41315, AT5G41410, AT5G42630, AT5G43270, AT5G45980, AT5G47220, AT5G48670, AT5G51990, AT5G52830, AT5G53200, AT5G53210, AT5G53950, AT5G54070, AT5G56110, AT5G56270, AT5G56860, AT5G59570, AT5G59820, AT5G60690, AT5G60890, AT5G60910, AT5G61270, AT5G61420, AT5G61850, AT5G62000, AT5G62020, AT5G62380, AT5G62430, AT5G65050, AT5G66870, AT5G67300, and AT5G67420.

In various embodiments, the engineered protein sensor and/or switch is an engineered version of a Drosophila melanogaster TF, such as, e.g., CG10325, CG11648, CG6093, CG3796, CG9151, CG15845, CG3935, CG3166, CG8376, CG3258, CG6677, CG3629, CG1034, CG3578, CG11491, CG12653, CG1759, CG6384, CG11924, CG4881, CG8367, CG17894, CG8669, CG2714, CG5893, CG9745, CG5102, CG2189, CG33183, CG9908, CG10798, CG1897, CG11094, CG2711, CG10604, CG32346, CG5714, CG1765, CG7383, CG32180, CG8127, CG1007, CG2988, CG9015, CG14941, CG8365, CG2328, CG8933, CG10488, CG6502, CG10002, CG2707, CG10034, CG2047, CG4059, CG33133, CG9656, CG2692, CG3388, CG7952, CG6494, CG11607, CG9786, CG4694, CG9768, CG1619, CG5748, CG17117, CG17835, CG2275, CG33956, CG10197, CG4717, CG4761, CG3340, CG3647, CG3758, CG4158, CG4148, CG7664, CG10699, CG5954, CG17743, CG1264, CG3839, CG32120, CG1689, CG8346, CG6096, CG8361, CG1705, CG14548, CG8328, CG8333, CG2050, CG18740, CG9045, CG10250, CG11450, CG6534, CG3851, CG1133, CG7467, CG6824, CG5109, CG12212, CG3978, CG17077, CG9610, CG8246, CG6716, CG7230, CG6348, CG10393, CG1849, CG9495, CG1030, CG8544, CG7734, CG1641, CG16738, CG3956, CG3836, CG11121, CG7847, CG3992, CG7938, CG17958, CG6993, CG8573, CG8599, CG8409, CG8068, CG11502, CG4216, CG16778, CG1378, CG6883, CG8651, CG1374, CG1856, CG10619, CG2956, CG10388, CG2762, CG4380, CG6172, CG7803, CG1046, CG1048, CG3411, CG12154, CG7895, CG3827, CG11387, CG17950, CG12287, CG7450, CG2368, CG6143, CG6338, CG2939, CG6464, CG17228, CG1322, CG1449, CG7672, CG14307, CG7771, CG5403, CG3497, CG5488, CG4220, CG2125, CG18412, CG7902, CG7937, CG18023, CG9097, CG2102, CG1130, CG3242, CG10021, CG1132, CG3668, CG11921, CG11922, CG9310, CG8887, CG3114, CG6634, CG1464, CG11049, CG14513, CG3090, CG8404, CG3886, CG12052, CG4354, CG1454, CG7018, CG5583, CG2914, CG4952, CG5683, CG4491, CG33152, CG9930, CG5441, CG6570, CG3905, CG8704, CG17921, CG4817, CG7562, CG2851, CG5965, CG7508, CG5580, CG5557, CG6964, CG5575, CG6794, CG2655, CG3052, CG6545, CG7187, CG17161, CG8625, CG12399, CG1775, CG1429, CG31240, CG7260, CG5529, CG4654, CG12223, CG6376, CG5247, CG11494, CG33261, CG12296, CG8103, CG1072, CG7959, CG7960, CG8567, CG18389, CG11992, CG5069, CG12245, CG10601, CG6103, CG1864, CG2678, CG5264, CG11987, CG6215, CG8522, CG7199, CG11783, CG8396, CG11798, CG9019, CG4029, CG10036, CG7951, CG7659, CG1650, CG10159, CG15319, CG5838, CG9398, CG7413, CG5393, CG10571, CG10605, CG14029, CG6604, CG17888, CG13598, CG4257, CG13951, CG9648, CG11186, CG3858, CG9696, CG5799, CG14938, CG1343, CG6312, CG5201, CG10052, CG8013, CG1447, CG32788, CG11202, CG9415, CG1507, CG10270, CG3998, CG5005, CG10269, CG7391, CG8667, CG8727, CG5206, CG13316, CG7807, CG2819, CG3848, CG16902, CG6269, CG10016, CG7760, CG9653, CG1414, CG15552, CG4013, CG8524, CG1071, CG5649, CG2712, CG1605, CG11182, CG18455, CG4303, CG9102, CG17829, CG2932, CG11551, CG2262, CG8474, CG6352, CG6121, CG7958, CG4143, CG11354, CG5935, CG8290, CG32575, CG9418, CG11352, CG3871, CG6627, CG1024, CG8108, CG2790, CG1966, CG11194, CG9776, CG7758, CG8208, CG2244, CG5067, CG5229, CG18783, CG18124, CG15286, CG11405, CG3268, CG11902, CG5133, CG15269, CG3491, CG17328, CG4185, CG16863, CG12630, CG32904, CG17594, CG1922, CG13906, CG18024, CG9233, CG12690, CG2875, CG17592, CG4136, CG12236, CG3726, CG3815, CG3847, CG14441, CG14438, CG3075, CG4575, CG3032, CG4617, CG9650, CG2116, CG2120, CG2129, CG15336, CG10959, CG18262, CG11294, CG12075, CG15365, CG7041, CG7055, CG2889, CG9817, CG2202, CG11122, CG11696, CG11695, CG11085, CG4404, CG4318, CG15749, CG1716, CG11172, CG11071, CG6211, CG9215, CG8119, CG8944, CG8578, CG8909, CG8924, CG9609, CG6769, CG5927, CG6470, CG7101, CG7556, CG14200, CG9571, CG11710, CG1529, CG11617, CG4133, CG31670, CG11723, CG17257, CG3407, CG17612, CG15435, CG15436, CG9088, CG13775, CG9200, CG4496, CG3838, CG13123, CG18619, CG18144, CG5034, CG12299, CG4621, CG6686, CG6792, CG9932, CG5204, CG9305, CG7099, CG5953, CG17912, CG5545, CG10348, CG10431, CG10446, CG17568, CG10263, CG10366, CG10462, CG10447, CG10631, CG10949, CG9342, CG18362, CG15216, CG1832, CG3136, CG2682, CG1845, CG1621, CG1620, CG1603, CG1602, CG12769, CG11641, CG8643, CG8216, CG1663, CG18446, CG12744, CG1407, CG18011, CG12942, CG12391, CG13204, CG12370, CG8821, CG8819, CG3850, CG4676, CG6061, CG6701, CG17385, CG17390, CG10209, CG8089, CG8092, CG16801, CG8314, CG8388, CG7786, CG4282, CG15710, CG17287, CG18468, CG4903, CG15073, CG11906, CG13424, CG9954, CG10543, CG9437, CG10321, CG10318, CG13493, CG11301, CG10344, CG9895, CG9890, CG9876, CG3941, CG5591, CG3065, CG3328, CG11414, CG4707, CG6905, CG1233, CG17181, CG13897, CG9139, CG2199, CG12104, CG1244, CG15812, CG14962, CG14965, CG12029, CG12605, CG15011, CG5249, CG17334, CG13287, CG13296, CG10274, CG7386, CG10147, CG8591, CG7404, CG7015, CG6683, CG6765, CG5093, CG5187, CG3891, CG3445, CG3654, CG7839, CG6272, CG11799, CG7368, CG4328, CG10704, CG10654, CG14117, CG17361, CG17359, CG7345, CG3919, CG6854, CG13458, CG7372, CG15715, CG9705, CG32171, CG18265, CG7271, CG4076, CG8765, CG11456, CG10565, CG7204, CG11247, CG14451, CG14655, CG14667, CG12162, CG10979, CG10296, CG9727, CG10267, CG33323, CG2702, CG9638, CG7963, CG8145, CG11762, CG8159, CG9793, CG9797, CG8359, CG11966, CG11984, CG11033, CG12952, CG16779, CG8301, CG8319, CG16899, CG8478, CG8484, CG6254, CG4570, CG4820, CG6689, CG6791, CG14710, CG6808, CG14711, CG6813, CG18476, CG6913, CG10042, CG5196, CG5245, CG33976, CG7518, CG15889, CG3143, CG7987, CG14860, CG6654, CG6276, CG5083, CG10278, CG5952, CG10309, CG3995, CG17803, CG17806, CG17802, CG17801, CG7357, CG7785, CG18599, CG7691, CG17186, CG4424, CG4854, CG4413, CG4936, CG4360, CG4217, CG15696, CG5737, CG7056, CG7045, CG7046, CG6990, CG4677, CG33336, CG4374, CG6129, CG5669, CG13617, CG13624, CG6892, CG11375, CG10669, CG4553, CG4730, CG17198, CG17197, CG17195, CG4956, CG32474, CG3350, CG5586, CG1647, CG14514, CG15504, CG15514, CG7928, CG2229, CG12071, CG11317, CG12054, CG1792, CG2052, CG11093, CG11152, CG11153, CG17172, CG6889, CG3743, CG13475, CG3526, CG11398, CG12767, CG15367, CG33473, CG14767, CG3576, CG12659, CG13109, CG12809, CG8817, CG8254, CG16910, CG3274, CG18764, CG32139, CG32577, CG2380, CG15736, CG13399, CG4427, CG12219, CG18647, CG31753, CG33720, CG30011, CG30020, CG30077, CG30401, CG30403, CG30420, CG30431, CG30443, CG31169, CG31224, CG31365, CG31388, CG31392, CG31441, CG31460, CG31481, CG31510, CG31612, CG31632, CG31642, CG31782, CG31835, CG31875, CG31955, CG32006, CG32050, CG32105, CG32121, CG32264, CG32296, CG32532, CG32719, CG32767, CG32772, CG32778, CG32830, CG33695, CG32982, CG33178, CG33213, CG33221, CG33520, CG33525, CG33557, CG33936, CG33980, CG34031, CG12632, CG17469, CG34100, CG34145, CG34149, CG34340, CG34346, CG34367, CG34376, CG34395, CG34403, CG34406, CG34407, CG34415, CG34419, CG34421, CG34422, CG8961, CG9397, CG10037, CG31258, CG31666, CG12196, CG6930, CG12238, CG33546, CG42234, CG34360, CG42267, CG42277, CG42281, CG42311, CG42332, CG42344, CG4807, CG7752, CG12701, CG17100, CG11971, CG42516, CG42515, CG6667, CG1028, CG3281, CG12124, CG42599, CG8506, CG17836, CG1070, and CG8676.

In various embodiments, the engineered protein sensor and/or switch is an engineered version of a mouse TF, such as, e.g., mouse loci 11538, 11568, 11569, 11614, 11622, 11624, 11632, 11634, 11694, 11695, 11733, 11736, 11819, 11835, 11859, 11863, 11864, 11865, 11878, 11906, 11908, 11909, 11910, 11911, 11920, 11921, 11922, 11923, 11924, 11925, 11991, 12013, 12014, 12020, 12021, 12022, 12023, 12029, 12051, 12053, 12142, 12151, 12173, 12180, 12189, 12192, 12224, 12265, 12326, 12355, 12387, 12393, 12394, 12395, 12399, 12400, 12416, 12417, 12418, 12454, 12455, 12566, 12567, 12572, 12578, 12579, 12580, 12581, 12590, 12591, 12592, 12606, 12607, 12608, 12609, 12611, 12653, 12677, 12705, 12753, 12785, 12848, 12912, 12913, 12914, 12915, 12916, 12951, 13017, 13018, 13047, 13048, 13134, 13163, 13170, 13172, 13180, 13196, 13198, 13345, 13390, 13392, 13393, 13394, 13395, 13396, 13433, 13435, 13486, 13494, 13496, 13555, 13557, 13559, 13560, 13591, 13592, 13593, 13626, 13653, 13654, 13655, 13656, 13661, 13709, 13710, 13711, 13712, 13713, 13714, 13716, 13796, 13797, 13798, 13799, 13813, 13819, 13864, 13865, 13871, 13872, 13875, 13876, 13982, 13983, 13984, 14008, 14009, 14011, 14013, 14025, 14028, 14029, 14030, 14055, 14056, 14085, 14105, 14106, 14154, 14155, 14200, 14233, 14234, 14235, 14236, 14237, 14238, 14239, 14240, 14241, 14247, 14281, 14282, 14283, 14284, 14359, 14390, 14391, 14457, 14460, 14461, 14462, 14463, 14464, 14465, 14472, 14489, 14531, 14534, 14536, 14581, 14582, 14605, 14632, 14633, 14634, 14659, 14797, 14815, 14836, 14842, 14843, 14884, 14885, 14886, 14896, 14912, 15110, 15111, 15161, 15163, 15181, 15182, 15183, 15184, 15185, 15193, 15205, 15206, 15207, 15208, 15209, 15213, 15214, 15218, 15220, 15221, 15223, 15227, 15228, 15229, 15242, 15248, 15251, 15258, 15260, 15273, 15284, 15285, 15331, 15353, 15354, 15361, 15364, 15370, 15371, 15372, 15373, 15375, 15376, 15377, 15378, 15379, 15384, 15394, 15395, 15396, 15397, 15398, 15399, 15400, 15401, 15402, 15403, 15404, 15405, 15407, 15408, 15410, 15412, 15413, 15414, 15415, 15416, 15417, 15421, 15422, 15423, 15424, 15425, 15426, 15427, 15429, 15430, 15431, 15432, 15433, 15434, 15436, 15437, 15438, 15460, 15499, 15500, 15563, 15569, 15900, 15901, 15902, 15903, 15904, 15951, 15976, 16150, 16151, 16201, 16348, 16362, 16363, 16364, 16371, 16372, 16373, 16391, 16392, 16476, 16477, 16478, 16596, 16597, 16598, 16599, 16600, 16601, 16656, 16658, 16761, 16764, 16814, 16815, 16825, 16826, 16842, 16869, 16870, 16871, 16872, 16873, 16874, 16875, 16876, 16909, 16911, 16917, 16918, 16969, 17095, 17119, 17121, 17122, 17125, 17126, 17127, 17128, 17129, 17130, 17131, 17132, 17133, 17134, 17135, 17172, 17173, 17187, 17188, 17191, 17192, 17215, 17216, 17217, 17218, 17219, 17220, 17257, 17258, 17259, 17260, 17261, 17268, 17274, 17283, 17285, 17286, 17300, 17301, 17318, 17341, 17342, 17344, 17354, 17355, 17420, 17425, 17428, 17480, 17536, 17537, 17681, 17684, 17692, 17701, 17702, 17703, 17749, 17764, 17765, 17859, 17863, 17864, 17865, 17869, 17870, 17876, 17877, 17878, 17927, 17928, 17932, 17933, 17936, 17937, 17938, 17977, 17978, 17979, 17984, 18002, 18012, 18013, 18014, 18018, 18019, 18020, 18021, 18022, 18023, 18024, 18025, 18027, 18028, 18029, 18030, 18032, 18033, 18034, 18036, 18037, 18038, 18044, 18045, 18046, 18071, 18072, 18088, 18089, 18091, 18092, 18094, 18095, 18096, 18109, 18124, 18128, 18129, 18131, 18132, 18140, 18142, 18143, 18171, 18181, 18185, 18193, 18198, 18227, 18291, 18292, 18393, 18412, 18420, 18423, 18424, 18426, 18432, 18503, 18504, 18505, 18506, 18507, 18508, 18509, 18510, 18511, 18514, 18515, 18516, 18519, 18572, 18606, 18609, 18612, 18616, 18617, 18626, 18627, 18628, 18667, 18676, 18685, 18736, 18740, 18741, 18742, 18771, 18789, 18854, 18933, 18935, 18983, 18985, 18986, 18987, 18988, 18990, 18991, 18992, 18993, 18994, 18995, 18996, 18997, 18998, 18999, 19009, 19013, 19014, 19015, 19016, 19017, 19018, 19049, 19056, 19060, 19084, 19099, 19127, 19130, 19182, 19184, 19202, 19213, 19231, 19290, 19291, 19326, 19330, 19377, 19401, 19411, 19434, 19645, 19650, 19651, 19664, 19668, 19687, 19696, 19697, 19698, 19708, 19712, 19724, 19725, 19726, 19727, 19763, 19820, 19822, 19826, 19883, 19885, 20016, 20017, 20018, 20019, 20020, 20021, 20022, 20024, 20128, 20174, 20181, 20182, 20183, 20185, 20186, 20204, 20218, 20220, 20230, 20231, 20232, 20289, 20371, 20375, 20384, 20409, 20429, 20439, 20464, 20465, 20466, 20467, 20471, 20472, 20473, 20474, 20475, 20476, 20480, 20481, 20583, 20585, 20586, 20587, 20589, 20591, 20592, 20602, 20613, 20638, 20664, 20665, 20666, 20667, 20668, 20669, 20670, 20671, 20672, 20673, 20674, 20675, 20677, 20678, 20679, 20680, 20681, 20682, 20683, 20687, 20688, 20689, 20728, 20787, 20788, 20807, 20819, 20833, 20841, 20842, 20846, 20847, 20848, 20849, 20850, 20851, 20852, 20893, 20901, 20904, 20922, 20923, 20924, 20997, 21339, 21340, 21341, 21343, 21349, 21350, 21374, 21375, 21380, 21382, 21383, 21384, 21385, 21386, 21387, 21388, 21389, 21399, 21400, 21401, 21405, 21406, 21407, 21408, 21410, 21411, 21412, 21413, 21414, 21415, 21416, 21417, 21418, 21419, 21420, 21422, 21423, 21425, 21426, 21427, 21428, 21429, 21652, 21674, 21676, 21677, 21678, 21679, 21685, 21780, 21781, 21783, 21804, 21807, 21815, 21833, 21834, 21835, 21843, 21847, 21848, 21849, 21869, 21885, 21886, 21887, 21888, 21907, 21908, 21909, 21917, 21929, 21945, 21981, 22025, 22026, 22051, 22057, 22059, 22061, 22062, 22088, 22160, 22200, 22221, 22255, 22259, 22260, 22278, 22282, 22286, 22326, 22337, 22383, 22385, 22431, 22433, 22608, 22632, 22634, 22639, 22640, 22642, 22646, 22654, 22658, 22661, 22666, 22668, 22678, 22680, 22685, 22689, 22691, 22694, 22695, 22696, 22697, 22698, 22700, 22701, 22702, 22704, 22709, 22710, 22712, 22715, 22717, 22718, 22719, 22722, 22750, 22751, 22754, 22755, 22756, 22757, 22758, 22759, 22761, 22762, 22764, 22767, 22768, 22770, 22771, 22772, 22773, 22775, 22776, 22778, 22779, 22780, 23808, 23827, 23849, 23850, 23856, 23857, 23871, 23872, 23885, 23894, 23942, 23957, 23958, 23989, 23994, 24068, 24074, 24075, 24113, 24116, 24135, 24136, 26356, 26371, 26379, 26380, 26381, 26386, 26404, 26413, 26417, 26419, 26423, 26424, 26427, 26461, 26465, 26573, 26754, 26927, 26939, 27049, 27056, 27057, 27059, 27081, 27140, 27217, 27223, 27224, 27274, 27386, 28019, 29806, 29808, 29813, 29861, 29871, 30046, 30051, 30794, 30841, 30923, 30927, 30928, 30942, 30944, 30946, 30951, 50496, 50524, 50721, 50754, 50777, 50783, 50794, 50796, 50817, 50868, 50887, 50907, 50913, 50914, 50916, 50996, 51792, 51813, 52024, 52040, 52231, 52502, 52609, 52615, 52705, 52708, 52712, 52897, 53314, 53317, 53357, 53380, 53415, 53417, 53626, 53868, 53869, 53970, 53975, 54006, 54123, 54131, 54132, 54139, 54169, 54343, 54352, 54388, 54422, 54446, 54562, 54601, 54633, 54678, 54711, 55927, 55942, 55994, 56030, 56070, 56196, 56198, 56218, 56220, 56222, 56233, 56275, 56309, 56312, 56314, 56321, 56353, 56380, 56381, 56404, 56406, 56449, 56458, 56469, 56484, 56490, 56501, 56503, 56505, 56522, 56523, 56525, 56613, 56642, 56707, 56736, 56771, 56784, 56787, 56805, 56809, 56856, 56869, 57080, 57230, 57246, 57314, 57316, 57376, 57737, 57745, 57748, 57756, 57765, 57782, 58172, 58180, 58198, 58202, 58206, 58234, 58805, 59004, 59021, 59024, 59026, 59035, 59057, 59058, 60345, 60406, 60611, 64050, 64144, 64290, 64379, 64383, 64384, 64406, 64453, 64685, 65020, 65247, 65255, 65256, 65257, 66056, 66118, 66136, 66213, 66233, 66277, 66352, 66376, 66420, 66464, 66491, 66505, 66556, 66596, 66622, 66634, 66642, 66671, 66698, 66729, 66799, 66867, 66880, 66923, 66930, 66959, 66970, 66980, 66985, 67057, 67065, 67122, 67150, 67151, 67155, 67199, 67235, 67260, 67279, 67288, 67367, 67370, 67379, 67381, 67389, 67419, 67439, 67575, 67657, 67673, 67692, 67710, 67815, 67847, 67873, 67949, 67985, 67993, 68040, 68153, 68196, 68268, 68346, 68479, 68558, 68701, 68705, 68776, 68839, 68842, 68854, 68910, 68911, 68992, 69020, 69125, 69167, 69168, 69188, 69234, 69241, 69257, 69260, 69299, 69317, 69389, 69539, 69606, 69656, 69716, 69790, 69833, 69890, 69920, 69944, 70073, 70122, 70127, 70315, 70350, 70392, 70408, 70428, 70459, 70497, 70508, 70601, 70625, 70637, 70650, 70673, 70779, 70796, 70797, 70823, 70859, 70981, 71041, 71063, 71131, 71137, 71163, 71176, 71241, 71280, 71371, 71375, 71409, 71458, 71468, 71592, 71597, 71702, 71722, 71752, 71767, 71777, 71782, 71793, 71828, 71834, 71838, 71839, 71939, 71949, 71990, 71991, 72057, 72074, 72135, 72180, 72195, 72199, 72290, 72293, 72323, 72325, 72388, 72459, 72465, 72475, 72556, 72567, 72615, 72720, 72727, 72739, 72823, 72949, 72958, 73178, 73181, 73340, 73389, 73451, 73469, 73503, 73610, 73614, 73844, 73845, 73945, 74007, 74068, 74106, 74120, 74123, 74149, 74164, 74168, 74197, 74282, 74318, 74322, 74326, 74335, 74352, 74377, 74481, 74533, 74561, 74570, 74838, 75196, 75199, 75210, 75291, 75305, 75339, 75387, 75480, 75482, 75507, 75572, 75599, 75605, 75646, 75725, 75901, 76007, 76022, 76294, 76308, 76365, 76389, 76467, 76572, 76580, 76793, 76803, 76804, 76834, 76893, 76900, 77057, 77114, 77117, 77264, 77286, 77318, 77480, 77683, 77889, 77907, 77913, 78020, 78088, 78246, 78251, 78284, 78455, 78469, 78541, 78619, 78656, 78699, 78703, 78783, 78829, 78910, 78912, 78921, 78929, 79221, 79233, 79362, 79401, 80283, 80509, 80720, 80732, 80859, 80902, 81601, 81630, 81703, 81845, 81879, 83383, 83395, 83396, 83557, 83602, 83925, 83993, 84653, 93674, 93681, 93686, 93691, 93759, 93760, 93761, 93762, 93837, 93871, 94047, 94112, 94187, 94275, 96979, 97064, 97165, 98053, 98403, 99377, 99730, 100090, 100563, 100710, 100978, 101095, 101206, 102162, 102209, 102334, 103136, 103806, 103889, 104328, 104349, 104360, 104383, 104384, 104394, 104886, 105377, 105594, 105859, 106795, 106894, 107351, 107433, 107499, 107503, 107568, 107586, 107751, 107765, 107771, 107889, 107932, 107951, 108060, 108098, 108143, 108655, 108672, 108857, 109113, 109115, 109575, 109594, 109663, 109676, 109889, 109910, 109958, 109972, 109973, 109995, 110052, 110061, 110068, 110109, 110147, 110506, 110521, 110616, 110641, 110647, 110648, 110784, 110796, 110805, 110913, 112077, 114142, 114565, 114606, 114642, 114774, 114889, 116810, 116848, 116870, 116871, 116912, 117168, 117198, 117590, 118445, 140477, 140490, 140500, 140577, 140743, 170574, 170644, 170729, 170740, 170767, 170787, 170791, 170826, 170938, 192195, 192231, 192285, 192651, 192657, 193796, 195333, 208076, 208258, 208266, 208292, 208439, 208677, 208715, 209011, 209357, 209361, 209416, 209446, 209448, 209707, 210135, 210162, 211378, 212168, 212276, 212391, 212712, 213010, 213990, 214105, 214162, 214384, 214669, 214899, 215031, 216151, 216154, 216285, 216456, 216558, 216578, 217031, 217082, 217127, 217166, 217558, 218030, 218440, 218490, 218624, 218772, 218989, 219150, 223227, 223690, 223701, 223922, 224419, 224585, 224656, 224694, 224829, 224902, 224903, 225876, 225895, 225998, 226049, 226182, 226442, 226641, 226747, 226896, 227099, 227644, 227656, 227940, 228136, 228598, 228731, 228775, 228790, 228829, 228839, 228852, 228869, 228876, 228880, 228980, 229004, 229534, 229663, 229906, 230073, 230162, 230587, 230674, 230700, 230753, 230908, 230936, 230991, 231044, 231051, 231329, 231386, 231986, 231991, 232232, 232337, 232807, 232853, 232854, 232878, 232906, 233056, 233410, 233490, 233863, 233887, 233890, 233908, 233987, 234725, 234959, 235028, 235041, 235050, 235320, 235442, 235582, 235623, 235682, 236193, 237052, 237336, 237409, 237615, 237758, 237960, 238247, 239099, 239546, 239652, 240064, 240120, 240263, 240427, 240442, 240476, 240590, 240690, 241066, 241447, 241520, 242523, 242620, 242705, 243187, 243833, 243906, 243931, 243963, 243983, 244349, 244713, 244813, 244954, 245572, 245583, 245596, 245688, 245841, 246086, 246196, 246198, 246791, 252829, 260298, 268281, 268301, 268396, 268448, 268564, 268741, 268903, 268932, 269252, 269713, 269870, 270076, 270627, 271278, 271305, 272347, 272359, 272382, 277353, 319196, 319207, 319535, 319594, 319599, 319601, 319615, 319695, 319785, 320067, 320376, 320429, 320586, 320595, 320790, 320799, 320875, 320995, 328572, 330301, 330361, 330502, 332937, 338353, 347691, 353187, 353208, 378435, 381319, 386626, and 386655.

Illustrative aTFs are found in Ramos, et al. Microbiology and Molecular Biology Reviews, June 2005, p. 326-356 and Tropell, et al. Microbiol Mol Biol Rev. 2004 September; 68(3):474-500, the contents of which are hereby incorporated by reference in their entireties.

In some embodiments, protein sensor amino acid sequences upon which engineering is to occur may, in various embodiments, be selected by sequence homology using one or more of BLASTP, PSI-BLAST, DELTA-BLAST, OR HMMER, JackHMMER, or the corresponding nucleotide sequences selected by sequence homology search.

Methods of identifying protein sequences that can be candidate protein sensors are found in US 2016/0063177, the entire contents of which are hereby incorporated by reference in its entirety.

In some embodiments, engineering approaches that alter the binding activity of a wild type allosteric protein sensor, e.g., engineering the wild type allosteric protein sensor to be suitable for binding the target molecule at the expense of the allosteric proteins cognate ligand (i.e., the ligand that binds to the wild type allosteric protein sensor) include mutagenesis. In some embodiments, mutagenesis comprises introducing one or more amino acid mutations in the wild type allosteric protein sensor, e.g., independently selected from substitutions, insertions, deletions, and truncations.

In some embodiments, the amino acid mutations are amino acid substitutions, and may include conservative and/or non-conservative substitutions.

“Conservative substitutions” may be made, for instance, on the basis of similarity in polarity, charge, size, solubility, hydrophobicity, hydrophilicity, and/or the amphipathic nature of the amino acid residues involved. The 20 naturally occurring amino acids can be grouped into the following six standard amino acid groups: (1) hydrophobic: Met, Ala, Val, Leu, Ile; (2) neutral hydrophilic: Cys, Ser, Thr; Asn, Gln; (3) acidic: Asp, Glu; (4) basic: His, Lys, Arg; (5) residues that influence chain orientation: Gly, Pro; and (6) aromatic: Trp, Tyr, Phe.

As used herein, “conservative substitutions” are defined as exchanges of an amino acid by another amino acid listed within the same group of the six standard amino acid groups shown above. For example, the exchange of Asp by Glu retains one negative charge in the so modified polypeptide. In addition, glycine and proline may be substituted for one another based on their ability to disrupt α-helices.

As used herein, “non-conservative substitutions” are defined as exchanges of an amino acid by another amino acid listed in a different group of the six standard amino acid groups (1) to (6) shown above.

In some embodiments, the substitutions may also include non-classical amino acids (e.g. selenocysteine, pyrrolysine, N-formylmethionine β-alanine, GABA and δ-Aminolevulinic acid, 4-aminobenzoic acid (PABA), D-isomers of the common amino acids, 2,4-diaminobutyric acid, α-amino isobutyric acid, 4-aminobutyric acid, Abu, 2-amino butyric acid, γ-Abu, ε-Ahx, 6-amino hexanoic acid, Aib, 2-amino isobutyric acid, 3-amino propionic acid, ornithine, norleucine, norvaline, hydroxyproline, sarcosme, citrulline, homocitrulline, cysteic acid, t-butylglycine, t-butylalanine, phenylglycine, cyclohexylalanine, β-alanine, fluoro-amino acids, designer amino acids such as β methyl amino acids, C α-methyl amino acids, N α-methyl amino acids, and amino acid analogs in general).

In some embodiments, the engineered protein sensor is engineered using design from existing allosteric proteins, e.g., aTFs. In some embodiments, the designing comprises in silico design. Illustrative, non-limiting, design principles are found in US 2016/0063177, the entire contents of which are hereby incorporated by reference in their entirety.

For example, in some embodiments, molecular modeling is used to predict mutations in an allosteric protein which may render the allosteric protein able to bind one or more target molecules. In various embodiments, reference to an experimentally derived three-dimensional protein structure, typically obtained through experimental methods including, but not limited to, x-ray crystallography, nuclear magnetic resonance (NMR), scattering, or diffraction techniques, is employed to model and/or predict mutations in an allosteric protein which may render the allosteric protein able to bind one or more target molecule. In various embodiments, the ROSETTA software suite is employed to assist with modelling (see Kaufmann et al. Biochemistry. 2010 Apr. 13; 49(14):2987-98, the entire contents of which are hereby incorporated by reference in its entirety). Alternatively, or in combination, a homology modeling algorithm such as ROBETTA, TASSER, I-TASSER, HHpred, HHsearch, or MODELLER, or SWISS-MODEL can be used. In some embodiments, such as (without limitation) those in which allosteric protein lacks an experimentally derived three-dimensional protein structure, a homology modeling algorithm can be used to build the sequence homology models. In various embodiments, one or more sequence or structural homologs have less than 90% amino acid sequence identity, less than 85% amino acid sequence identity, less than 80% amino acid sequence identity, less than 75% amino acid sequence identity, less than 70% amino acid sequence identity, less than 65% amino acid sequence identity, less than 60% amino acid sequence identity, less than 55% amino acid sequence identity, less than 50% amino acid sequence identity, less than 45% amino acid sequence identity, less than 40% amino acid sequence identity, less than 35% amino acid sequence identity, less than 30% amino acid sequence identity, less than 25% amino acid sequence identity, or less amino acid sequence identity to the amino acid sequence of the three-dimensional protein structure. Illustrative homology modelling methods and principles are found in US 2016/0063177, e.g. at paragraphs [0085]-[0093], the entire contents of which are hereby incorporated by reference in its entirety.

In some embodiments, a structure of a wild type allosteric protein is evaluated for alterations which may render the allosteric protein able to bind one or more target molecules (e.g. by docking a one or more target molecules into the structure of an allosteric protein). Illustrative docking methods and principles are found in US 2016/0063177, e.g. at paragraphs [0095]-[0101], the entire contents of which are hereby incorporated by reference in its entirety.

In various embodiments, libraries of potential mutations to wild type allosteric protein are made and selection, positive or negative, is used to screen desired mutants.

In various embodiments, engineering may use the technique of computational protein design (as disclosed in U.S. Pat. Nos. 7,574,306 and 8,340,951, which are hereby incorporated by reference in their entirety) directed evolution techniques, rational mutagenesis, or any suitable combination thereof.

In other embodiments, mutation techniques such as gene shuffling, homologous recombination, domain swapping, deep mutation scanning, and/or random mutagenesis may be employed.

Illustrative protein sensors and cognate ligands are found in WO 2015/127242, for instance in the table of page 7, the contents of which are hereby incorporated by reference in their entirety.

In various embodiments, the protein sensor is engineered using design from existing allosteric proteins, e.g. aTFs. In various embodiments, the designing comprises in silico design. Illustrative design principles are found in US 2016/0063177, the entire contents of which are hereby incorporated by reference in their entirety.

For example, in various embodiments, molecular modeling is used to predict mutations in an allosteric protein which may render the allosteric protein able to bind one or more target molecules. In various embodiments, reference to an experimentally derived three-dimensional protein structure, typically obtained through experimental methods including, but not limited to, x-ray crystallography, nuclear magnetic resonance (NMR), scattering, or diffraction techniques, is employed to model and/or predict mutations in an allosteric protein which may render the allosteric protein able to bind one or more target molecule. In various embodiments, the ROSETTA software suite is employed to assist with modelling (see Kaufmann et al. Biochemistry. 2010 Apr. 13; 49(14):2987-98, the entire contents of which are hereby incorporated by reference in its entirety).

Alternatively, or in combination, a homology modeling algorithm such as ROBETTA, TASSER, I-TASSER, HHpred, HHsearch, or MODELLER, or SWISS-MODEL can be used. In some embodiments, such as (without limitation) those in which allosteric protein lacks an experimentally derived three-dimensional protein structure, a homology modeling algorithm can be used to build the sequence homology models. In various embodiments, one or more sequence or structural homologs have less than 90% amino acid sequence identity, less than 85% amino acid sequence identity, less than 80% amino acid sequence identity, less than 75% amino acid sequence identity, less than 70% amino acid sequence identity, less than 65% amino acid sequence identity, less than 60% amino acid sequence identity, less than 55% amino acid sequence identity, less than 50% amino acid sequence identity, less than 45% amino acid sequence identity, less than 40% amino acid sequence identity, less than 35% amino acid sequence identity, less than 30% amino acid sequence identity, less than 25% amino acid sequence identity, or less amino acid sequence identity to the amino acid sequence of the three-dimensional protein structure. Illustrative homology modelling methods and principles are found in US 2016/0063177, e.g. at paragraphs [0085]-[0093], the entire contents of which are hereby incorporated by reference in its entirety.

In some embodiments, a structure of an allosteric protein is evaluated for alterations which may render the allosteric protein able to bind one or more target molecules (e.g. by docking a one or more target molecules into the structure of an allosteric protein). Illustrative docking methods and principles are found in US 2016/0063177, e.g. at paragraphs [0095]-[0101], the entire contents of which are hereby incorporated by reference in its entirety.

In various embodiments, libraries of potential mutations to the allosteric protein are made and selection, positive or negative, is used to screen desired mutants.

In various embodiments, engineering may use the technique of computational protein design (as disclosed in U.S. Pat. Nos. 7,574,306 and 8,340,951, which are hereby incorporated by reference in their entirety) directed evolution techniques, rational mutagenesis, or any suitable combination thereof.

In other embodiments, mutation techniques such as gene shuffling, homologous recombination, domain swapping, deep mutation scanning, and/or random mutagenesis may be employed.

By way of example, but not by way of limitation, Table 1 provides illustrative protein sensors that may be modified in accordance with various embodiments of the present invention. For instance, in various embodiments, one may select an aTF (“Chassis”) and/or native ligand and make reference to a provided representative structure (PDB) to, in accordance with the disclosure herein, design an engineered protein sensor to a target molecule or class of target molecules (see Target Molecule Property column).

TABLE 1

| aTF | | | Representative | Target Molecule |
|---|---|---|---|---|
| (“Chassis”) | Native Ligand | Native Host | Structure (PDB) | Property |
| QscR | Bound to N-3-oxo- | Psudemonas | 3SZT | long chain fatty acids and |
| | dodecanoyl-L- | aeruginosa | | homoserine lactones |
| | Homoserine | | | |
| | Lactone | | | |
| NtcA | 2-oxoglutarate, | Anabaena | 3LA2, LA3, 3LA7 | 3-7 carbon acids/ |
| | 2,2- | cyanobacterium | | alcohols |
| | difluoropentanoic | | | |
| | acid | | | |
| CarH | adenosylcobalamin | Thermus | 5C8A, 5C8D, 5C8E, | cobalamine |
| | | thermophilus | 5C8F | |
| CcpN | ADP | Bacillus subtilis | 3FV6, 3FWR, 3FWS | nucleotides, nucleosides |
| repressor | | | | |
| BtAraR | arabinose | Bacteriodes | 5BS6, 5DD4, 5DDG, | saccharides |
| | | thetaiotaomicron | 5DEQ | |
| AraR | arabinose | Bacteroides | 5BS6, 5DD4, 5DDG, | saccharides |
| | | thetaiotaomicron VPI | 5DEQ | |
| AhrR | Arginine | Bacillus subtilis | 2P5L 2P5M | charged amino acids, |
| | | | | quanidino groups |
| Rv1846c | betalactams | Mycobacterium | 2G9W | betalactams |
| | | tuberculosis. | | |
| CviR | C6 HSL | Chromobacterium | 3QP1, 3QP2, 3QP4, | short chain fatty acids |
| | | violaceum | 3QP5, 3QP6, 3QP8 | and homoserine lactones |
| MtbCRP | cAMP | Myco tuberculosis | 3I54 | cyclic nucleotides |
| BmrR | cationic antibiotics, | Bacillus subtilis | 3Q1M, 3Q2Y, 3Q3D, | cationic multirings |
| | dyes, and | | 3Q5P, 3Q5R, 3Q5S | |
| | disinfectants | | | |
| Rrf2 | cysteine | Bacillus subtilis | 2Y75 | hydrophobic amino acids, |
| | | | | sulfur containing |
| | | | | molecules |
| CGL2612 | drugs | Corynebacterium | 1V7B, 2ZOY | rigid multiring molecules |
| | | glutamicum | | |
| TtgR | drugs | Pseudomonas | 2UXH, 2UXI, 2UXO, | rigid multiring molecules |
| | | putida | 2UXP, 2UXU | |
| QacR | Ethidium, | Staphylococcus | 3BR3 3BR6 2DTZ | chemically rigid, bivalent |
| | rhodamine, | Aureus | 2HQ5 | compounds. |
| Cra | fructose 1 | Pseudomonas | 3O74, 3O75 | sugar phosphates |
| | phosphate | putida | | |
| GabR | gamma- | Bacillus subtilis | 4N0B | short chain amines and |
| | aminobutyric acid | | | acids |
| YvoA | glucosamine-6- | Bacillus subtilis | 4U0V, 4U0W, 4U0Y, | C5, C6 sugars |
| | phosphate, | | 4WWC | |
| | acetylglucosamine- | | | |
| | 6-phosphate | | | |
| CggR | glucose-6- | Bacillus subtilis | 2OKG, 3BXE, 3BXF, | C5, C6 sugars |
| | phosphate and | | 3BXG, 3BXH | |
| | fructose-6- | | Also Cited By: 4OQP, | |
| | phosphate | | 4OQQ | |
| CodY | GTP, Isoleucine | Bacillus subtilis | 2B0L, 2B18, 2GX5, | hydrophobic amino acids |
| | | | 2HGV | nucleosids, nucleotides, |
| | | | | nucleotide phosphates |
| HrcA | heat | Thermotoga | 1STZ | temperature, useful for |
| | | maritima | | circular |
| | | | | permutation/stability |
| | | | | measurements |
| RovA | heat | Yersinia pestis | 4AIH, 4AIJ, 4AIK | temperature, useful for |
| | | | | circular |
| | | | | permutation/stability |
| | | | | measurements |
| LldR | lactose | Corynebacterium | 2DI3 | saccharides |
| (CGL2915) | | glutamicum | | |
| LacI | Lactose/IPTG | E. coli | 2p9h | saccharides |
| NMB0573/ | leucine methionine | Neisseria | 2P5V, 2P6S, 2P6T | hydrophobic amino acids, |
| AsnC | | meningitidis | | sulfer containing |
| | | | | compounds |
| FapR | malonyl-CoA | Bacillus subtilis | 2F3X, 2F41 | c3-c7 molecules, CoA |
| | | | | cofactors |
| FapR | malonyl-CoA | Staphylococcus | 4A0X, 4A0Y, 4A0Z, | c3-c7 molecules, CoA |
| | | Aureus | 4A12 | cofactors |
| LmrR | MDR pump | Lactococcus lactis | 3F8B, 3F8C, 3F8F | rigid multiring molecules |
| | controller | | | |
| SMET | MDR pump | Stenotrophomonas | 2W53 | rigid multiring molecules |
| | controller | maltophilia | | |
| SCO4008 | methylene blue, | Streptomyces | 2D6Y | |
| | crystal | coelicolor | | |
| | violetcationic | | | |
| | antibiotics, dyes, | | | |
| | and disinfectants | | | |
| MntR | Mn2+ | Bacillus subtilis | 4hv6 | metals and cations |
| Rex | NADH | Bacillus subtilis, | 2VT2, 2VT3 | cofactors |
| | | Thermus | | |
| | | thermophilus, | | |
| | | Thermus aquaticus | | |
| NikR | Nickle | Helobacter pylori | 3PHT, 3QSI, 2WVB | |
| DNR | NO (via heme) | Pseudomonas | 2Z69 | metals and cations |
| | | aeruginosa | | |
| FadR | oleoyl-CoA | Vibrio cholerae | 4P96, 4P9U, 4PDK | long chain fatty acids and |
| | | | | cofactors |
| MosR | oxidative state | Mycobacterium | 4FX0, 4FX4 | oxidative state, useful for |
| | | tuberculosis. | | circular permutation |
| OhrR | oxidative state | Bacillus subtilis | 1Z91, 1Z9C | oxidative state, useful for |
| | (cys) | | | circular permutation |
| SarZ | oxidative stress | Staphylococcus | 3HRM, 3HSE, 3HSR | oxidative state, useful for |
| | | Aureus | | circular permutation |
| TsaR | para- | Comamonas | 3FXQ, 3FXR, 3FXU, | c6-c12 aromatics |
| | toluensulfonate | testosteroni | 3FZJ | |
| HetR | PatS | Anabaena sp. | 4YNL, 4YRV | peptides and proteins |
| NprR | peptide | Bacillus thuringiensis | 4GPK | peptides and proteins |
| MexR | peptide | Pseudomonas | 3ECH | peptides and proteins |
| | | aeruginosa | | |
| PhoP | PhoR | Mycobacterium | 2PMU | peptides and proteins |
| | | tuberculosis. | | |
| PurR | Phosphoribosyl- | Bacillus subtilis | 1P4A | phosphorilated sugars |
| | pyrophosphate | | | |
| PcaV | protocatechuate (a | Streptomyces | 4FHT, 4G9Y | aromatic acids, c4-c10 |
| (SCO6704) | dihyroxy benzoic | coelicolor | | acids |
| | acid) | | | |
| DesR | self His-PO4 | Bacillus subtilis | 4LDZ, 4LE0, 4LE1, | useful for circular |
| | | | 4LE2 | permutation |
| SinR | sinL dimer? | Bacillus subtilis | 2YAL, 3QQ6 | peptides and proteins |
| EthR | something | Mycobacterium | 1T56 | c4-c20 hydrophobic |
| | hydrophobic | tuberculosis. | | molecules |
| BlcR | succinate | Agrobacterium | 3MQ0 | short chain aldehydes |
| | semialdehyde | tumefaciens | | |
| TetR-class | Tet | Pasteurella | 2VPR | rigid multiring molecules |
| H | | multocida | | |
| TetR | Tetracycline | E. coli Tn10 | 4AC0 | rigid multiring molecules |
| TreR | trehalose | Bacillus subtilis | 2OGG | saccharides |
| DntR | TsaR type LTTR | Burkholderia cepacia | 5AE3, 5AE4 | c6-c12 aromatics |
| HyIIIR | unknown large | Bacillus cereus | 2FX0 | large moledules |
| | molecule | | | |
| CprB | γ-butyrolactones | Streptomyces | 4PXI | short chain lactones |
| | | coelicolor | | |
| AcuR | acrylic acid | Rhodobacter | 3BRU | Short chain acid and |
| | | sphaeroides | | hydrocarbons |

In various embodiments, the amino acids targeted for mutation or in silico design are those within about 3, or about 5, or about 7, or about 10, or about 12 Angstroms (e.g. between about 3 to about 12 Angstroms, or between about 5 to about 12 Angstroms, or between about 7 to about 12 Angstroms, or between about 10 to about 12 Angstroms, or between about 3 to about 5 Angstroms, or between about 3 to about 7 Angstroms, or between about 3 to about 10 Angstroms) of a ligand modeled into a binding pocket either through docking or by experimental methods such as X-ray crystallography.

Mutated allosteric proteins that may be protein sensors and/or switches able to bind one or more target molecules can be screen using standard binding assays (e.g. fluorescent, radioactive assays, etc.).

In various embodiments, the engineered protein sensor is engineered as described in Taylor, et al. Nat. Methods 13(2): 177, the entire contents of which are hereby incorporated by reference in its entirety.

Engineered Producer Strains/Cells

The engineered producer strains (or cells) described above refer to strains or cells (e.g., bacterial, yeast, algal, plant, insect, or mammalian (human or non-human) strains or cells) that have been engineered to produce at least one target product (or molecule) of interest, wherein the target product (or molecule) of interest is capable of being detected by the sensor system discussed above (e.g., detection by an engineered sensor plasmid or strain).

By way of example, in some embodiment, the target product (or molecule) of interest, for which an engineered protein sensor may be engineered include one or more of the compounds described in WO 2015/017866, e.g. at paragraphs [00107]-[00112], the entire contents of which are hereby incorporated by reference in its entirety.

In some embodiments, the target molecules of the present technology are toxic to a cell and/or cannot be readily bind or interact with an engineered protein sensor in a detectable manner in a cellular environment. In some embodiments, the engineered protein sensor is selected based on its cognate ligand identity and any commonality the cognate ligand may have with a target molecule. For example, in some embodiments, a shared chemical group between a cognate ligand and a target molecule may direct one to the engineered protein sensor that binds to the cognate ligand and lead to the engineering of the protein sensor so it can bind to the target molecule.

By way of example, but not by way of limitation, Table 1 (above) provides illustrative target molecule or class of target molecules (see Target Molecule Property column).

In some embodiments, the target molecule (or product) is naringenin.

Reporters

In some embodiments, useful reporters in the present technology include proteins with unique spectral signatures, such as, without limitation, green fluorescent protein whose expression may be determined by measuring its adsorbance or fluorescence using a microtiter plate fluorimeter, fluorescent microscope, visual inspection, or a fluorescence activated cell sorter (FACS). In some embodiments, reporters also include, without limitation, spectral signatures based on adsorbance, physical properties such as magnetism and impedance, changes in redox state, assayable enzymatic activities, such as a phosphatase, beta-galactosidase, peroxidase, luciferase, or gas generating enzymes. Alternatively, in some embodiments, a linear single or double stranded DNA that encodes the reporter and transcription factor library member may be used as a reporter in cases not limited to amplification by polymerases.

In some embodiments, the present technology includes a reporter gene system, which comprises a protein having a unique spectral signature and/or assayable enzymatic activity. Illustrative reporter systems or detection methods include, but are not limited to, those using chemiluminescent or fluorescent proteins, such as, for example, green fluorescent protein (GFP), enhanced green fluorescent protein (EGFP), Renilla Reniformis green fluorescent protein, GFPmut2, GFPuv4, yellow fluorescent protein (YFP), enhanced yellow fluorescent protein (EYFP), cyan fluorescent protein (CFP), enhanced cyan fluorescent protein (ECFP), enhanced blue fluorescent protein (EBFP), chromoproteins, citrine and red fluorescent protein from discosoma (dsRED), infrared fluorescent proteins, luciferase, umbelliferone, rhodamine, fluorescein, dichlorotriazinylamine fluorescein, dansyl chloride, phycoerythrin, and the like. Examples of detectable bioluminescent proteins include, but are not limited to, luciferase (e.g., bacterial, firefly, click beetle and the like), luciferin, aequorin and the like. Examples of detectable enzyme systems include, but are not limited to, galactosidases, glucorinidases, phosphatases, peroxidases, cholinesterases, proteases, and the like. In certain other embodiments, the reporter systems detection methods include an enzyme. In certain other embodiments, the detectable marker is a non-essential gene that can be assayed rapidly for genetic variation by qPCR. In certain other embodiments, the detectable marker is a drug resistance marker that can be readily assessed for functionality by reverse selection. In some embodiments, the detectable marker is a nutritional marker, e.g. production of a required metabolite in an auxotrophic strain, ability to grow on a sole carbon source, or any other growth selection strategy known in the art.

In certain embodiments, the reporter is composed of two or more components which when present together produce the functional reporter. Examples include split GFPs, and enzymes such as luciferase, beta galactosidase, beta lactamase, and dihydrofolate reductase. One or more components of a split reporter may be introduced exogenously allowing detection of cellular production of fewer components. The split reporter may be used to detect a complementing split reporter-fused to another protein allowing detection either inside the cell, outside the cell, or both. For instance, a split GFP fusion protein may be excreted by a cell encapsulated with the complementing reporter component such that the producing cell does not have the capacity to produce a functional reporter until encapsulated with its complement. One or more components of such split systems may be produced independently and added as a detection reagent to the cells being assayed.

For example, beta-glucosidase and Antarctic phosphatase may be used as reporter systems with their corresponding fluorogenic substrates fluorescein di-(β-D-glucopyranoside) and fluorescein diphosphate.

In some embodiments, the binding event of the aTF itself is utilized to present a physical readout of aTF state through either optical or non-optical methods in an acellular environment. For example, in a non-limiting example, the aTF is linked to a fluorescent protein and the DNA binding site is linked to a quencher molecule. Fluorescent readout is possible only when the aTF is released from the DNA binding site itself. This method allows for a direct readout of aTF binding events. This strategy is not limited to fluorophore quencher pairs, but may also employ other read outs such as split proteins. Additionally, the binding event may be used to physically separate functional proteins from non-functional proteins in the case of protein display methods.

Host Strains/Cells

In various embodiments, the host cells (i.e., sensor strains/cells and producer strains/cells) of the present technology include eukaryotic and/or prokaryotic cells, including bacterial, yeast, algal, plant, insect, mammalian cells (human or non-human), and immortal cell lines.

By way of example, in some embodiments, the host cell may be Escherichia coli, Saccharomyces cerevisiae, Pichia pastoris, Saccharomyces castellii, Kluyveromyces lactis, Pichia stipitis, Schizosaccharomyces pombe, Chlamydomonas reinhardtii, Arabidopsis thaliana, or Caenorhabditis elegans. In some embodiments the host cell is a bacterial cell, such as Escherichia spp., Streptomyces spp., Zymonas spp., Acetobacter spp., Citrobacter spp., Synechocystis spp., Rhizobium spp., Clostridium spp., Corynebacterium spp., Streptococcus spp., Xanthomonas spp., Lactobacillus spp., Lactococcus spp., Bacillus spp., Pedobacter spp., Bacteroides spp., Alcaligenes spp., Pseudomonas spp., Aeromonas spp., Azotobacter spp., Comamonas spp., Mycobacterium spp., Rhodococcus spp., Gluconobacter spp., Ralstonia spp., Acidithiobacillus spp., Microlunatus spp., Geobacter spp., Geobacillus spp., Arthrobacter spp., Flavobacterium spp., Serratia spp., Saccharopolyspora spp., Thermus spp., Stenotrophomonas spp., Chromobacterium spp., Sinorhizobium spp., Saccharopolyspora spp., Agrobacterium spp. and Pantoea spp. The bacterial cell can be a Gram-negative cell such as an E. coli, or a Gram-positive cell such as a species of Bacillus.

In some embodiments, the cell is a fungal cell such as a yeast cell, e.g., Saccharomyces spp., Schizosaccharomyces spp., Pichia spp., Paffia spp., Kluyveromyces spp., Candida spp., Talaromyces spp., Brettanomyces spp., Pachysolen spp., Debaryomyces spp., Yarrowia spp., and industrial polyploid yeast strains. Preferably the yeast strain is a S. cerevisiae strain or a Yarrowia spp. strain. Other examples of fungi include Aspergillus spp., Pennicilium spp., Fusarium spp., Rhizopus spp., Acremonium spp., Neurospora spp., Sordaria spp., Magnaporthe spp., Allomyces spp., Ustilago spp., Botrytis spp., and Trichoderma spp.

In some embodiments, the cell is an algal cell or a plant cell (e.g., A. thaliana, C. reinhardtii, Arthrospira, P. tricomutum, N. tabacum, T. suecica, P. carterae, P. tricomutum, Chlorella spp., such as Chlorella vulgaris).

Target cells can include transgenic and recombinant cell lines. In addition, heterologous cell lines can be used, such as Chinese Hamster Ovary cells (CHO).

In some embodiments, the host cell is an Actinomycetes spp. cell. Actinomycetes are a heterogeneous collection of bacteria that form branching filaments which include, for example, Actinomyces, Actinomadura, Nocardia, Streptomyces and related genera. In some embodiments, Actinomyces comprise Streptomyces. In some embodiments, the Actinomycetes spp. cell is a Streptomyces cell. (e.g. S. coelicolor). Streptomyces include, by way of non-limiting example, S. noursei, S. nodosus, S. natalensis, S. venezuelae, S. roseosporus, S. fradiae, S. lincolnensis, S. alboniger, S. griseus, S. rimosus, S. aureofaciens, S. clavuligerus, S. avermitilis, S. platensis, S. verticillus, S. hygroscopicus, and S. viridochromeogenes.

In some embodiments, the host cell is a Bacillus spp. cell. In some embodiments, the Bacillus spp. cell is selected from B. alcalophilus, B. alvei, B. aminovorans, B. amyloliquefaciens, B. aneurinolyticus, B. anthracis, B. aquaemaris, B. atrophaeus, B. boroniphilus, B. brevis, B. caldolyticus, B. centrosporus, B. cereus, B. circulans, B. coagulans, B. firmus, B. flavothermus, B. fusiformis, B. galliciensis, B. globigii, B. infemus, B. larvae, B. laterosporus, B. lentus, B. licheniformis, B. megaterium, B. mesentericus, B. mucilaginosus, B. mycoides, B. natto, B. pantothenticus, B. polymyxa, B. pseudoanthracis, B. pumilus, B. schlegelii, B. sphaericus, B. sporothermodurans, B. stearothermophilus, B. subtilis, B. thermoglucosidasius, B. thuringiensis, B. vulgatis, and B. weihenstephanensis.

Droplets for Engineered Sensor or Producer Strains/Cells

Due to high interfacial area of dispersed droplets, emulsions without emulsifiers are thermodynamically unstable systems. In order to stabilize emulsion droplets, low molar mass surfactants or surface-active polymers usually have to be included in the formulations to decrease the interfacial tension between the phases. One way to stabilize droplets is by using solid particles (e.g., nanoparticles) to replace the surfactants. Solid particles accumulate at the interface between two immiscible fluids or liquids and build a rigid barrier against coalescence. The solid particles reduce or prevent coalescence, which brings about higher stability to emulsions. Similar to an egg shell, the dense layer of solid particles makes a rigid crust so that emulsion droplets resist coalescence.

Pickering emulsion is an emulsion that is stabilized by solid particles in place of an emulsifier. Pickering emulsions possess many unique features that classical emulsions stabilized by surfactants do not, such as superior stability and low toxicity.

As disclosed herein, the methods of the present disclosure comprise a droplet that is surrounded by an immiscible continuous phase that comprises a fluorinated-based oil or emulsion. In some embodiments of the present technology, the emulsion is a Pickering emulsion. The droplets can then be assayed for levels of a target molecule, wherein an engineered protein sensor provides a readout of the level of a target molecule produced by the engineered producer cell. The methods of the present disclosure include isolating the droplets with isolated engineered producer cells that produce desired levels of the target molecule; breaking the droplets encapsulating isolated engineered producer cells that produce desired levels of the target molecule to form the population of engineered producer cells, wherein the population of engineered producer cells is an enriched population of engineered producer cells that produce desired levels of the target molecule.

In some embodiments, the pool of engineered producer cells is transformed with an engineered sensor plasmid.

In some embodiments, the methods comprise merging each droplet containing the engineered producer cell with a droplet encapsulating an engineered sensor cell, wherein the engineered sensor cell produces an engineered protein sensor.

In some embodiments, the immiscible continuous phase that surrounds the droplet is a fluorinated-based oil or emulsion. In some embodiments, the immiscible continuous phase that surrounds the droplet is an organic oil.

The fluorinated-based oil, in some embodiments, is a fluorinated oil, a fluorinated polymer, a water-in fluorocarbon emulsion, a water-in perfluorocarbon emulsion, or combinations thereof.

In some embodiments, the immiscible continuous phase that surrounds the droplet is an organic oil.

The Pickering emulsion can be stabilized in several ways. In some embodiments, the Pickering emulsion is stabilized by decreasing the chain length of the oil or organic oil. Upon decreasing the oil or organic oil chain length, the solubility of the oil or organic oil increases, allowing for the preparation of a stabilized Pickering emulsion.

In some embodiments, the fluorinated oil or emulsion is optionally stabilized by a particle. In some embodiments, the particle is a partially fluorinated nanoparticle. In some embodiments, the particle is a partially hydrophobic nanoparticle (e.g., a silica-based hydrophobic nanoparticle).

In some embodiments, the emulsion is a Pickering emulsion stabilized by a hydrocarbon (e.g., hexadecane, dodecane, decane). In some embodiments, the emulsion is a Pickering emulsion stabilized by an oil or organic oil, such as a mineral oil, a corn oil, or a castor oil.

In some embodiments, the emulsion is stabilized by an oil or organic oil combined with Tween, Triton X-100, Triton X-114, SPAN, Arlacel, a non-ionic emulsifier, such as ABIL, a detergent, or combinations thereof.

In some embodiments, the emulsion is stabilized by an oil or organic oil combined with a protein stabilizer (e.g., BSA, β-lactoglobulin, BCN). In some embodiments, the emulsion is stabilized by an oil or organic oil combined with a non-ionic detergent or sugar (e.g., glucose, fructose, lactose). In some embodiments, the protein stabilizer, non-ionic detergent or sugar reduce diffusion of organics from the second phase (e.g., an aqueous, organic, or droplet phase) into the first phase (e.g., the oil-based phase).

In some embodiments, the emulsion is stabilized by Tween, Triton X-100, Triton X-114, SPAN, Arlacel, a non-ionic emulsifier, such as ABIL, a detergent, or combinations thereof. In some embodiments, the emulsion is stabilized by a protein stabilizer (e.g., BSA, β-lactoglobulin, BCN). In some embodiments, the emulsion is stabilized by an oil or organic oil combined with a non-ionic detergent or sugar (e.g., glucose, fructose, lactose),In some embodiments, the Pickering emulsion accumulates at the interface between two immiscible phases. In some embodiments, the first phase is a continuous phase and the second phase is a dispersive phase. In some embodiments, the emulsion of the present disclosure comprises a first phase that is oil-based, such as a fluorocarbon phase, and a second phase (e.g., an organic, aqueous, droplet, hydrocarbon, or gas phase). For example, the first phase can be a fluorocarbon phase having at least one fluorinated solvent, and the second phase can be immiscible with the fluorinated solvent, such as an organic, aqueous, droplet, hydrocarbon, or a gas phase. In some embodiments, the second phase is an aqueous phase. In some embodiments, the second phase is a hydrocarbon phase.

In some embodiments, the droplet is under microfluidic control.

In some embodiments, the present disclosure relates to compositions and methods for producing droplets of fluid surrounded by a liquid. The fluid and the liquid may be essentially immiscible in many cases, e.g., immiscible on a time scale of interest (e.g., the time it takes a fluidic droplet to be transported through a particular system or device). The fluid may also contain other species, for example, certain molecular species, such as cells, particles, etc.

In some embodiments, a droplet is an isolated portion of a first fluid that is completely surrounded by a second fluid. It is to be noted that a droplet is not necessarily spherical, but may assume other shapes as well, for example, depending on the external environment. In some embodiments, the droplet has a minimum cross-sectional dimension that is substantially equal to the largest dimension of the channel perpendicular to fluid flow in which the droplet is located. In some embodiments, the term droplet may be used interchangeably with the term “microcapsule.”

In some embodiments, the droplets of the present disclosure are formed from emulsions (e.g., Pickering emulsions); systems of two immiscible fluid or liquid phases with one of the phases dispersed in the other, for example, as droplets of microscopic or colloidal size.

Emulsions may be produced from any suitable combination of immiscible liquids. For example, the emulsion disclosed herein can have an aqueous liquid and a hydrophobic, immiscible liquid, such as oil.

In some embodiments, disclosed herein are droplets formed from an emulsion of the present disclosure comprise (a) a continuous phase, and (b) at least one droplet dispersed in the continuous phase. In some embodiments, the emulsion comprises (a) a continuous fluorophilic phase, and (b) at least one dispersed aqueous or lipophilic phase dispersed in the continuous fluorophilic phase.

In some embodiments, the dispersed phase (e.g., aqueous, organic, hydrocarbon or gas phase) comprises at least one engineered producer cell. In some embodiments, the engineered producer cell is anchored to an amphiphilic particle (e.g., a silica-based nanoparticle) at the interface of the fluorous phase and the aqueous, organic, hydrocarbon or gas phase.

In some embodiments, the amphiphilic particles (e.g., a silica-based nanoparticles) and combinations thereof described herein provide sufficient stabilization against coalescence of droplets, without interfering with processes that can be carried out inside the droplets.

The emulsion may be stabilized by addition of one or more surface-active agents (surfactants). These surfactants are termed emulsifying agents and act at, for example, the water/oil interface to prevent (or at least delay) separation of the phases.

In some embodiments, the emulsion comprises a fluorocarbon (or perfluorocarbon) continuous phase. For example, stable water-in-perfluorooctyl and water-in-perfluorooctylethane emulsions can be formed using F-alkyl dimorpholinophosphates as surfactants. Non-fluorinated compounds are essentially insoluble in fluorocarbons and perfluorocarbons and small drug-like molecules (typically <500 Da and Log P<5) are compartmentalized very effectively in the aqueous microcapsules of water-in-fluorocarbon and water-in-perfluorocarbon emulsions—with little or no exchange between microcapsules (e.g., droplets).

In some embodiments, creation of an emulsion generally requires the application of mechanical energy to force the phases together. There are a variety of ways of doing this which utilize a variety of mechanical devices, including stirrers (such as magnetic stir-bars, propeller and turbine stirrers, paddle devices and whisks), homogenizers (including rotor-stator homogenizers, high-pressure valve homogenizers and jet homogenizers), colloid mills, ultrasound and ‘membrane emulsification’ devices, and microfluidic devices.

In some embodiments, complicated biochemical processes, notably gene transcription and translation are also active in aqueous phase microcapsules, as disclosed herein, which are formed in water-in-oil emulsions. This can enable compartmentalization in water-in-oil emulsions for the selection of genes, which are transcribed and translated in emulsion microcapsules and selected by the binding or catalytic activities of the proteins they encode. Aqueous microcapsules formed in the emulsion are generally stable with little if any exchange of nucleic acids, proteins, or the products of enzyme catalyzed reactions between microcapsules. In some embodiments of the present disclosure, the technology exists to create emulsions with volumes all the way up to industrial scales of thousands of liters.

In some embodiments, a “microcapsule” can be a droplet of one fluid in a different fluid, where the confined components are soluble in the droplet, but not in the carrier fluid. In some embodiments there is a third material defining a wall, such as a membrane. In some embodiments, a microcapsule is an artificial compartment whose delimiting borders restrict the exchange of the components of the molecular mechanisms described herein which allow the sorting of the genetic elements according to the function of the gene products which they encode. In some embodiments, the term “microcapsule” may be used interchangeably with the term “droplet.”

In some embodiments, the droplet is under microfluidic control. In some embodiments, the microfluidic control comprises a microfluidic system having microfluidic channels that direct or otherwise control the formation and/or movement of droplets in order to carry out the methods disclosed herein. For example, in some embodiments, “microfluidic control” of droplet formation refers to the creation of droplets using a microfluidic device to form “droplets” of fluid within a second fluid. In some embodiments, droplets sorted under microfluidic control are sorted, as described herein, using a microfluidic device to perform one or more of the functions associated with the sorting procedure.

In some embodiments, the droplet is under microfluidic control, and the microfluidic control comprises a microfluidic system having microfluidic channels, wherein the channel has a feature that at least partially directs the flow of a fluid. The channel can have any cross-sectional shape (circular, oval, triangular, irregular, square or rectangular, or the like) and can be covered or uncovered. In some embodiments the channel can be completely covered, or at least one portion of the channel can have a cross-section that is completely enclosed, or the entire channel may be completely enclosed along its entire length with the exception of its inlet(s) and outlet(s). A channel may also have an aspect ratio (length to average cross sectional dimension) of at least 2:1, more typically at least 3:1, 5:1, or 10:1 or more. An open channel includes characteristics that facilitate control over fluid transport, e.g., structural characteristics (an elongated indentation) and/or physical or chemical characteristics (hydrophobicity vs. hydrophilicity) or other characteristics that can exert a force (e.g., a containing force) on a fluid. In some embodiments, the fluid within the channel may partially or completely fill the channel. In some embodiments, an open channel is used, and the fluid may be held within the channel, for example, using surface tension (e.g., a concave or convex meniscus). The channel may be of any size, for example, having a largest dimension perpendicular to fluid flow of less than about 5 mm or 2 mm, or less than about 1 mm, or less than about 500 microns, less than about 200 microns, less than about 100 microns, less than about 60 microns, less than about 50 microns, less than about 40 microns, less than about 30 microns, less than about 25 microns, less than about 10 microns, less than about 3 microns, less than about 1 micron, less than about 300 nm, less than about 100 nm, less than about 30 nm, or less than about 10 nm. In some embodiments, the dimensions of the channel may be chosen such that fluid is able to freely flow through the article or substrate. The dimensions of the channel may also be chosen, for example, to allow a certain volumetric or linear flowrate of fluid in the channel. Of course, the number of channels and the shape of the channels can be varied by any method known to those of ordinary skill in the art. In some embodiments, more than one channel or capillary may be used. For example, two or more channels may be used, where they are positioned inside each other, positioned adjacent to each other, positioned to intersect with each other, etc.

In some embodiments, the droplets of the present disclosure are formed from emulsions (e.g., Pickering emulsions); systems of two immiscible fluid or liquid phases with one of the phases dispersed in the other, for example, as droplets of microscopic or colloidal size. In some embodiments, a fluid is a liquid and the terms are interchangeable. In some embodiments, the fluid may have any suitable viscosity that permits flow. If two or more fluids are present, each fluid may be independently selected among essentially any fluids (liquids, gases, and the like) by those of ordinary skill in the art, by considering the relationship between the fluids. In some embodiments, the fluids may each be miscible or immiscible. For example, two fluids can be selected to be immiscible within the time frame of formation of a stream of fluids, or within the time frame of reaction or interaction. Where the portions remain liquid for a significant period of time then the fluids should be significantly immiscible. Where, after contact and/or formation, the dispersed portions can be quickly hardened by polymerization or the like, the fluids need not be as immiscible. Those of ordinary skill in the art can select suitable miscible or immiscible fluids, using contact angle measurements or the like, to carry out the techniques of the invention.

In some embodiments, the methods disclosed herein relate to producing a population of engineered producer cells in a plurality of droplets. In some embodiments, the plurality of droplets comprises at least one non-immortal cell. In some embodiments, the methods involve determining a characteristic of a species secreted by the non-immortal cell within the droplet, as disclosed in U.S. Patent Publication No. US2009/0068170, the contents of which are incorporated herein in their entirety.

In some embodiments, the methods disclosed herein relate to producing a population of engineered producer cells in a plurality of aqueous droplets, wherein each droplet is uniform in size and comprises droplet libraries that are useful to perform large numbers of assays while consuming only limited amounts of reagents, as disclosed in U.S. Patent Publication No. US2010/0022414, the contents of which are incorporated herein in their entirety.

In some embodiments, the methods disclosed herein relate to producing a population of engineered producer cells in an emulsion library comprising a plurality of aqueous droplets, as disclosed in U.S. Patent Publication No. US2017/0028365, the contents of which are incorporated herein in their entirety.

In some embodiments, the methods disclosed herein relate to producing a population of engineered producer cells in a droplet-based assay that is controlled and/or calibrated using signals detected from droplets, as disclosed in U.S. Patent Publication No. US2013/0084572, the contents of which are incorporated herein in their entirety.

In some embodiments, the methods disclosed herein relate to producing a population of engineered producer cells, comprising detecting droplets in a system having a detector device comprising an input flow path, an intersection region, and an output flow path, as disclosed in U.S. Patent Publication No. 2014/0179544, the contents of which are incorporated herein in their entirety.

In some embodiments, the methods disclosed herein relate to producing a population of engineered producer cells in a droplet, and detecting microfluidic droplets and particles within the droplets, as well as sorting the droplets, as disclosed in U.S. Patent Publication No. 201/80104693, the contents of which are incorporated herein in their entirety.

In some embodiments, the methods disclosed herein relate to producing a population of engineered producer cells in an emulsion, comprising: an aqueous dispersed phase; a continuous phase comprising a fluorinated oil; and a surfactant comprising a block copolymer that includes a perfluorinated polyether (PFPE)block coupled to a polyethylene glycol (PEG) block via an amide bond, wherein the surfactant comprises a formula —(CₙF₂ₙO)ₓ—(CₘF₂ₘ)_{y}—CONH— and n, m, x, and y are positive integers, as disclosed in U.S. Pat. No. 9,012,390, the contents of which are incorporated herein in their entirety.

The invention is further described with reference to the following non-limiting examples.

### Examples

### Example 1: Sensor Response Variation Across E. coli MG1655 Mutants

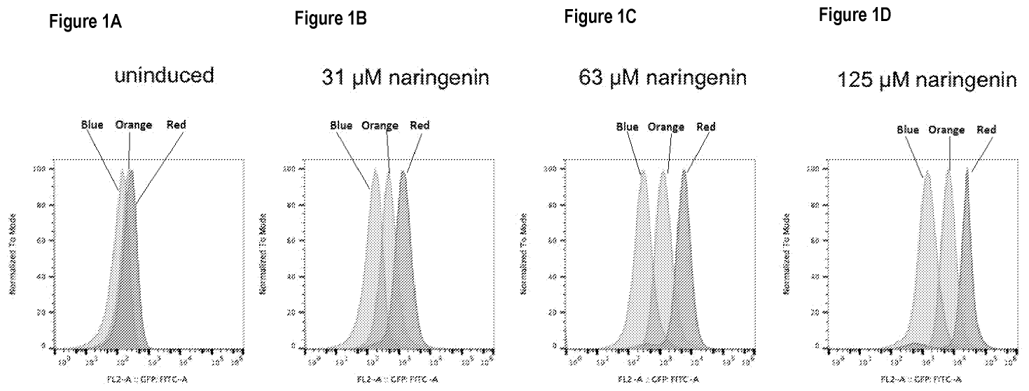

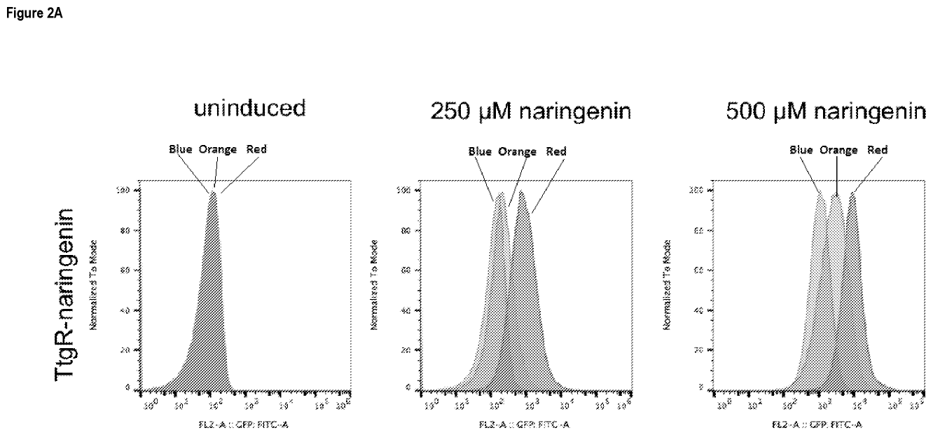

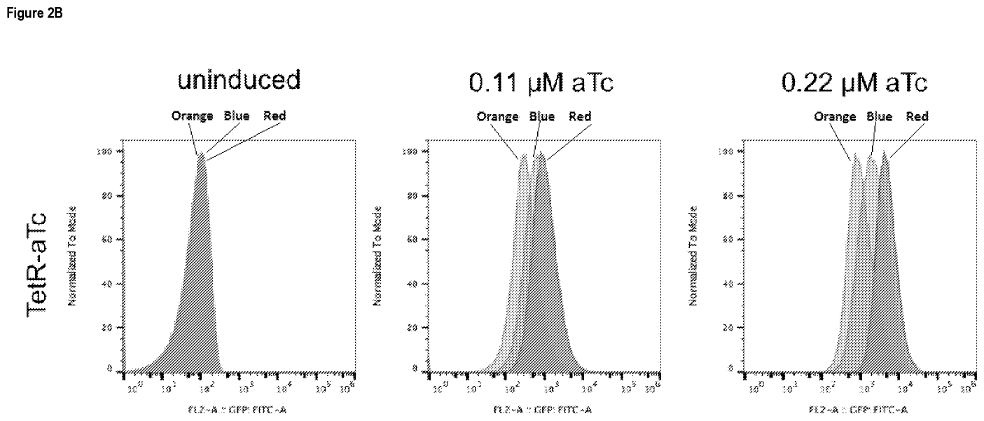

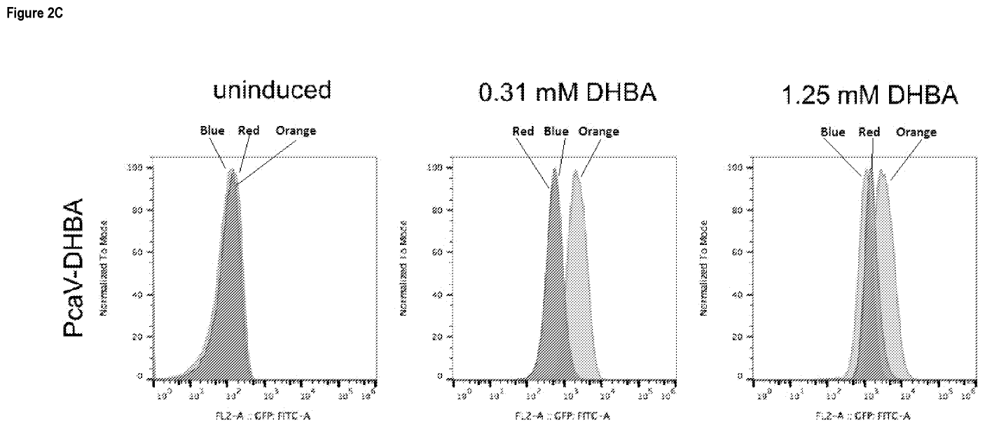

As an example of sensor variation across genomic mutants originating from the same background, FIGS. 1A-C shows the sensor response of three randomly selected members from a MAGE-engineered E. coli MG1655 population. The three clones were transformed with a medium-copy plasmid harboring TtgR and gfp under the control of an engineered ttgAp promoter. Exogenously applied naringenin (0, 31, 63, or 125 μM) induced different levels of GFP expression in the three strains. No naringenin pathway enzymes were present during these experiments, eliminating any interference by endogenously produced naringenin. The variation was not resolved upon transferring the sensor system to the genome (not shown) or switching to a high-copy sensor plasmid (FIG. 2A).

FIGS. 2A-D show variation in the sensor response of the same three MAGE-engineered E. coli MG1655 mutants from FIGS. 1A-C, which were transformed with high-copy plasmids harboring one of four aTFs (TtgR (FIG. 2A), TetR (FIG. 2B), PcaV (FIG. 2C), or QacR (FIG. 2D)) and gfp under the control of the appropriate aTF-regulated operator. Each sensor system was induced by different levels of the appropriate cognate ligand, as indicated in the figure. Strain colors are consistent across all panels. The trend in sensor response for the TtgR sensor system (red=brightest, orange=middle, blue=darkest) was not held across the different sensor systems, suggesting that the cause was not global.

### Example 2: Diffusion of Target Production Molecule Across Production Variants

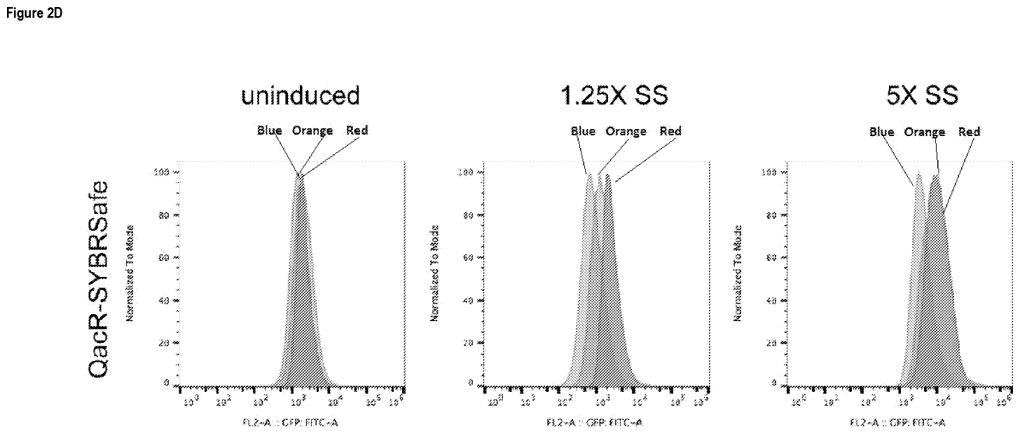

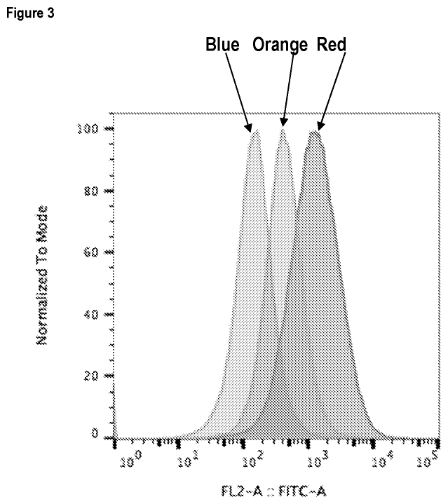

FIG. 3 demonstrates interference by diffusion across production strains. An E. coli K-12 MG1655 mutant, referred to as 2E6, was MAGE-engineered for enhanced naringenin precursor concentrations and transformed with two separate naringenin pathway plasmids: pNAR_{low} and pNAR_{high}, resulting in a high naringenin-producing strain (red, ˜180 μM production in a 24 h batch cultivation in M9 1% glucose) and a low naringenin-producing strain (blue, ˜60 μM production in a 24 h batch cultivation in M9 1% glucose). These two strains, which differed in their productivity were incubated separately and together (orange). The high- and low-producing strains show an averaged signal when cultured together, suggesting that naringenin diffusion across strains is prohibitive to screening for better producers in bulk liquid culture. Note that in this example, the difference in production is the result of plasmid-based engineering of naringenin pathway enzymes rather than large-scale genomic mutations to alter key metabolite concentrations. In these situations, the sensor response variation observed in Example 1 has not been observed.

### Example 3: Co-culturing as a Means to Separate Sensor from Producer

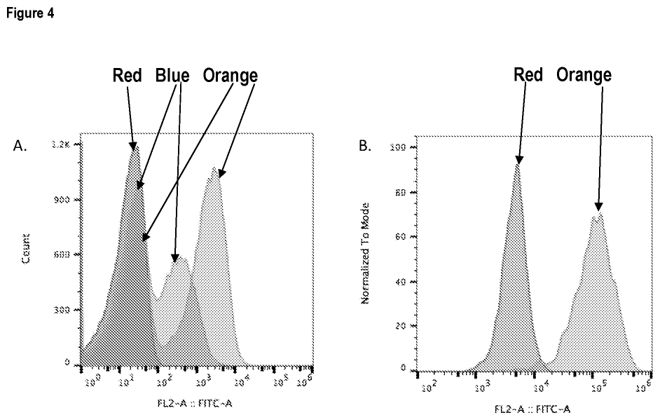

FIGS. 4A-B establish co-culturing of producer cells and sensor cells as a viable strategy for screening. Sensor cells were engineered in an E. coli BW25113 Δptsl::kanR background, which is unable to grow on glucose as a sole carbon source. The glucose transporter ptsl and fluorescence reporter gfp were expressed co-cistronically under the control of a TtgR-regulated promoter on the plasmid pSENSOR_{GFP-PtsI}, such that growth and the magnitude of GFP signal are naringenin-dependent. In FIG. 4A, sensor cells, which have naringenin-dependent growth and magnitude of GFP signal, were co-cultured in liquid with the producer strain 2E6, discussed in Example 2, transformed with three different naringenin pathway plasmids or control plasmid: a pathway negative control pNARₙᵤₗₗ (red), low naringenin-producing pathway pNAR_{low} (blue, ˜60 μM production in an isolated 24 h batch cultivation in M9 1% glucose), or high naringenin-producing pathway pNAR_{high} (orange, ˜180 μM production in a 24 h batch cultivation in M9 1% glucose). The dark population represents the producer cells, which have no GFP signal. The bright population represents the sensor cell population, which increases in ratio of the total co-cultured population and also in magnitude of GFP response with increasing production. In FIG. 4B, sensor cells were co-cultured with pathway negative control cells or high naringenin-producing cells in water-in-oil droplets. Following incubation, the water-in-oil droplets were encapsulated in another aqueous phase, generating water-in-oil-in-water droplets, which were observed on a standard fluorescence activated cell sorter (FACS). The distribution of the co-cultured high-producer and sensor cells (orange) is easily distinguished from the co-cultured non-producer and sensor cells (red).

### Example 4: Co-Culturing in Droplets

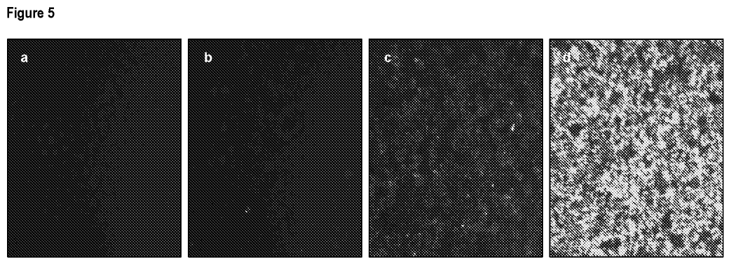

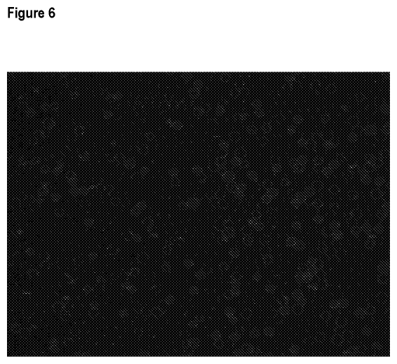

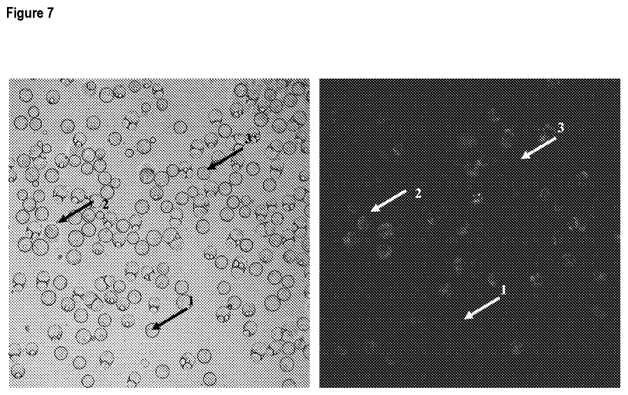

2E6 pNARₙᵤₗₗ, 2E6 pNAR^{low}, 2E6 pNAR_{high}, were co-cultured with the E. coli BW25113 Δptsl::kanR pSENSOR_{GFP-PtsI} sensor strain described in Example 3. The four cultures were grown overnight in LB medium, subdiluted 1 to 100 in minimal medium supplemented with the appropriate antibiotic and then grown into log phase. Once in log phase, the E. coli were rinsed 3× with 1× filtered M9 salts and then diluted to an OD₆₀₀ of 1.0. The 2E6 strains were diluted to an OD₆₀₀ of 0.01 to ensure that a single producer strain is present in each encapsulated droplet and the sensor cells were diluted to 0.1 to ensure that each droplet gets at least 5 sensor cells. Six sets of droplets were produced, (1) sensor cell only, (2) sensor cell with 500 μM naringenin, (3) 2E6 pNAR_{high} with sensor cell and 1 mM IPTG, (4) 2E6 pNARₙᵤₗₗ with sensor cell and 1 mM IPTG, (5), 2E6 pNAR_{low} with sensor cell and 1 mM IPTG, and (6) naringenin only droplets. The cell solution and oil phase composed of 1% Ran Fluorosurfactant in HFE7500 were loaded into 1 mL glass syringes and connected to two Harvard Apparatus syringe pumps. The liquids were emulsified using a 50 μm junction flow focusing, fluorophilic chip from Dolomite and at a rate of 14 and 10 μL/min respectively for the oil and aqueous phases. Formed droplets were collected in a 5 mL centrifuge tube. After formation, the droplets were incubated at 33° C. for 48 hours with tumbling. After incubation, the droplets were imaged under a microscope. Positive control droplets with 500 μM naringenin were very fluorescent (FIG. 5d), while the negative control droplets were dark (FIG. 5a). pNAR_{low} and pNAR_{high} co-cultured droplets produced detectable fluorescence (FIGS. 5b and c). 2E6 pNAR_{high} producers produce more naringenin that 2E6 pNAR_{low} producer cells and also resulted in brighter co-cultured droplets. Sensor cell droplets mixed with droplets containing 500 μM naringenin showed no diffusion between droplets (FIG. 6). Additional diffusion data analyzed by HPLC shows minimal naringenin and coumarate diffusion into the oil <20% (data not shown). After analysis, the single emulsion was converted to a bulk aqueous phase double emulsion using a 50 μM flow focusing, hydrophilic chip from Dolomite. To do so, the droplets were loaded into a 1 mL glass syringe and a second glass syringe was filled with fresh growth medium to be balanced isotonically with the droplet interior. The syringes were loaded onto two Harvard Apparatus syringe pumps and then connected to the chip. Double emulsions were formed at flow rates of 15 μL/min per syringe and collected in a 15 mL centrifuge tube. Double emulsions were analyzed under the microscope prior to FACS analysis. Three populations of droplets are visible under the microscope: (1) empty droplets, (2) droplets containing fluorescent sensor cells and producer cells, and (3) droplets containing non-fluorescent sensor cells and nonproducers (FIG. 7). FACS analysis of the droplets show three populations as well (FIG. 8A-C). Non-producer containing droplets demonstrate an average population fluorescence of ˜2,000 RFU (FIG. 8A). Producer containing droplets demonstrate an average population fluorescence of ˜100,000 RFU (FIG. 8B). A droplet population containing a mix of both strains demonstrate two distinct fluorescent subpopulations (FIG. 8C).

### Example 5: Abrogating Diffusion of Key Products Using Droplet Compartmentalization

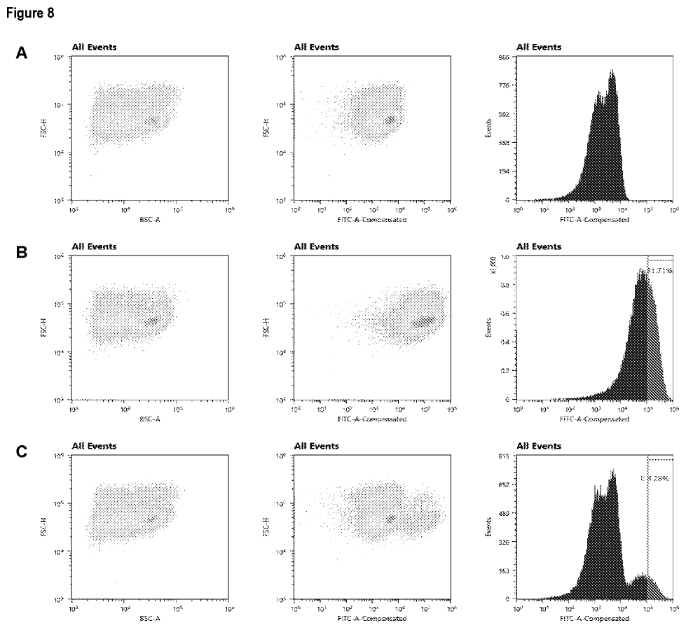

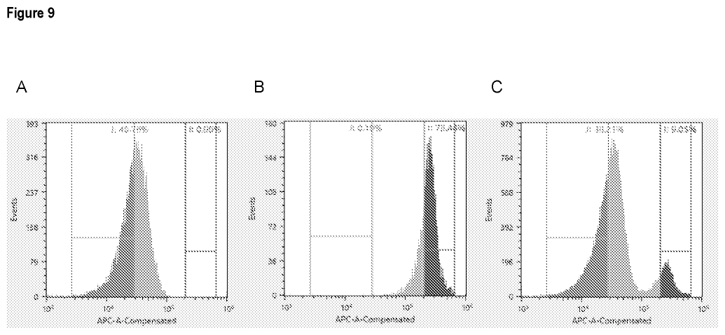

2E6 pNARₙᵤₗₗ and 2E6 pNAR_{high} were additionally transformed with the pSENSOR_{GFP} naringenin sensor system, which produces GFP in response to naringenin using a TtgR-derived sensor. The two cultures were grown overnight in LB medium, subdiluted 1 to 100 in minimal medium supplemented with the appropriate antibiotic and then grown into log phase. Once in log phase, the E. coli were rinsed 3× with 1× filtered M9 salts and then diluted to an OD₆₀₀ of 1.0. The 2E6 strains were then further diluted to an OD600 of 0.01 to ensure that a single producer strain is present in each encapsulated droplet. Three sets of droplets were produced (1) 2E6 pNAR_{high} cells only, (2) 2E6 pNARₙᵤₗₗ cells only, and (3) a 1:1 mixture of 2E6 pNAR_{high} and 2E6 pNARₙᵤₗₗ cells. The cell solution and oil phase composed of 1% Ran Fluorosurfactant in HFE7500 were loaded into 1 mL glass syringed and connected to two Harvard Apparatus syringe pumps. The liquids were emulsified using a 50 um junction flow focusing, fluorophilic chip from Dolomite and at a rate of 14 and 10 μL/min respectively for the oil and aqueous phases. Formed droplets were collected in a 5 mL centrifuge tube. After formation, droplets were incubated at 33° C. with tumbling for 48 hours. After incubation, droplets were broken using an equal volume of 1H,1H,2H,2H-Perfluoro-1-octanol vortexed for 30 s and then centrifuged to separate the phases. The aqueous phase containing the recovered E. coli was transferred to a fresh tube and then diluted for FACS analysis. The fluorescence distributions of the populations were measured by FACS. The 2E6 pNARₙᵤₗₗ cells demonstrated an average population fluorescence of ˜30,000 RFU (FIG. 9A). The 2E6 pNAR_{high} naringenin producer cells demonstrated an average population fluorescence of ˜300,000 RFU (FIG. 9B), 10× that of the non-producer strains. When grown together using droplets to isolate each producer strain, two fluorescent subpopulations are seen demonstrating the droplets ability to abrogate diffusion of the product and therefore population averaging (FIG. 9C).

FIG. 10 demonstrates enrichment of the high naringenin-producing strain 2E6 pNAR_{high} pSENSOR_{GFP} from the pathway negative control 2E6 pNARₙᵤₗₗ pSENSOR_{GFP}, following incubation in droplets to abrogate diffusion. Cells were cultured and washed as above, except that they were diluted to an OD600 of 0.003 to ensure single cell loading at the droplet size utilized in this experiment. Droplets were generated as described above except that the liquids were emulsified in a 25 μm junction flow focusing, PDMS chip manufactured in-house, at a rate of 20 and 12 μL/min respectively for the oil and aqueous phases. Separate droplet sets were generated for 2E6 pNAR_{high} pSENSOR_{GFP}, 2E6 pNARₙᵤₗₗ pSENSOR_{GFP}, and pre-mixed 2E6 pNAR_{high} pSENSOR_{GFP} and 2E6 pNARₙᵤₗₗ pSENSOR_{GFP}. Formed droplets were collected in a 5 mL centrifuge tube. After formation, droplets were incubated at 33° C. with tumbling for 24 hours. After incubation, droplets were broken using an equal volume of 1H,1H,2H,2H-Perfluoro-1-octanol, vortexed for 30 s, and then centrifuged to separate the phases. The aqueous phase containing the recovered E. coli was transferred to a fresh tube and then diluted for FACS analysis and cell sorting. Cells from the 2E6 pNAR_{high} pSENSOR_{GFP} (blue), 2E6 pNARₙᵤₗₗ pSENSOR_{GFP} (red), and mixed 2E6 pNAR_{high} pSENSOR_{GFP} and 2E6 pNARₙᵤₗₗ pSENSOR_{GFP} (orange) droplets were analyzed on a Bio-Rad S3E FACS. Following analysis, cells from the mixed 2E6 pNAR_{high} pSENSOR_{GFP} and 2E6 pNARₙᵤₗₗ pSENSOR_{GFP} droplets were sorted using the gating strategy depicted below. Sorted and unsorted cells were plated, and clones from the unsorted and the sorted populations were grown in production medium to determine the percentage of pathway+cells (determined by high GFP signal in the pathway+ and darkness in the pathway-clones). We observed a ˜7-fold enrichment of pathway+cells in this one enrichment cycle.

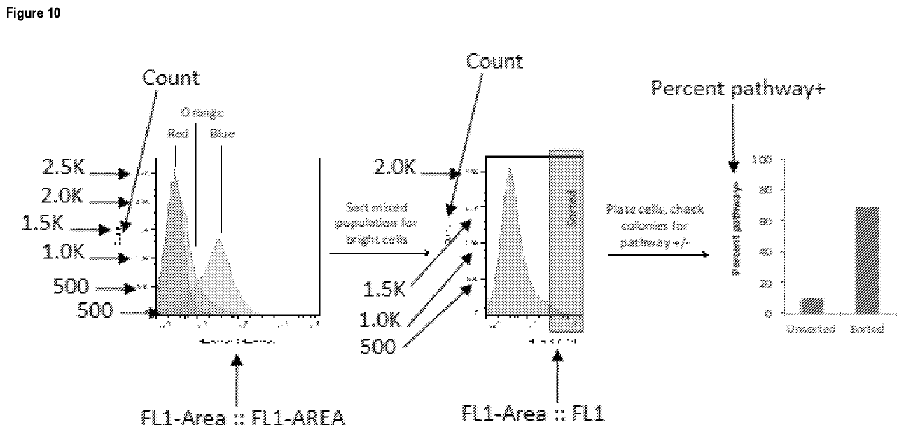

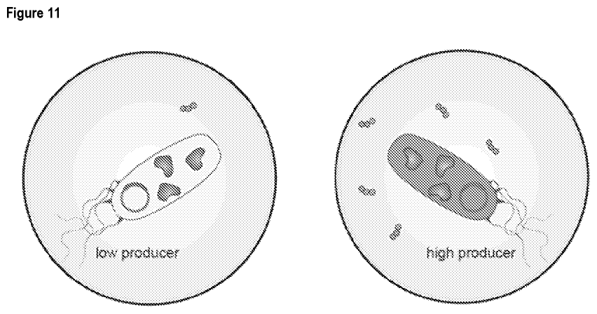

FIG. 11 shows a droplet encapsulation of a low- vs high-producer with the “sensor in cell”, where the same cell is responsible for both ligand and sensor production, and the fluorescent (green) read out intensity (in the high producer cell, right) is associated with the concentration of produced ligand.

### Example 6: Enrichment of a High Producers Using Co-Culture Sensor Cells

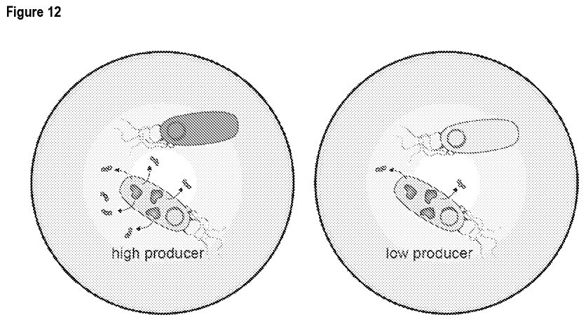

FIG. 12 depicts a system of “co-culture sensor cells” encapsulated with either a low- or high-producer in a droplet system. Here, only non-producing cells contribute to the sensor readout, reducing burden on producing cells, where the fluorescent (green) read out intensity (in the high producer cell, left) is associated with the concentration of produced ligand.

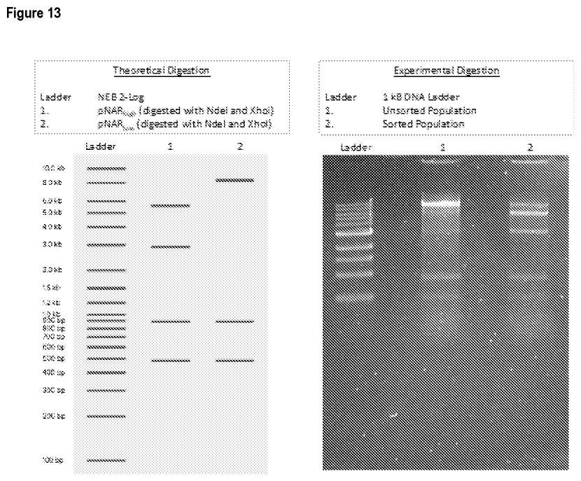

FIG. 13 demonstrates enrichment of the high naringenin-producing strain 2E6 pNAR_{high} from the low pathway control 2E6 pNAR_{low} utilizing a droplet co-culture strategy with Δptsi::kanR pSENSOR_{GFP-Ptsi} sensor strain described in Example 3. The three cultures were grown overnight in LB medium to stationary phase. Cells were then washed 1× with filtered 2×M9 media with 1% glucose, 0.1% pluronic F-68, and 1 mM IPTG. Cells were then resuspended in the same filtered media used for the wash, such that the Δptsi::kanR pSENSOR_{GFP-Ptsi} cells were resuspended at an OD₆₀₀ of 0.2. To this, either 2E6 pNAR_{high} or 2E6 pNAR_{low} were added at an OD₆₀₀ of 0.066, making two separate mixtures of low or high producing cells with equal concentration (OD₆₀₀=0.2) of sensor cells. Droplets were generated for each culture separately using HFE 7500+1% 008-FS as the oil phase and the preceding cell mixtures as the aqueous phase using a PDMS chip with 25 μm junction flow focusing, at a rate of 20 and 12 μL/min respectively for the oil and aqueous phases. After formation, droplets were incubated at 33° C. with tumbling for 24 hours. Prior to sorting, droplets were mixed at approximately [10]:[1] [Δptsi::kanR pSENSOR_{GFP-Ptsi}+2E6 pNAR_{low}]: [Δptsi::kanR pSENSOR_{GFP-Ptsi}+2E6 pNAR_{high}]. Droplets were then sorted on a PDMS sorter chip manufactured in-house with 45 μm height sorter chip with a 40 μm junction seated with indium electrodes capable of supplying high voltages (˜1 kV) to enact a dielectrophoretic effect for mobilizing aqueous droplets to a desired channel. Droplets were monitored at a 60× magnification, where the PMT voltage signal was assayed using in-house programmed microchips, such that droplets containing a signal greater than a user defined threshold would be sorted into the sorted channel by application of a 450 μs pulse of 800V at 10 kHz frequency and sorted. Afterwards, sorted and unsorted droplets were collected and broken using an equal volume of 1H,1H,2H,2H-Perfluoro-1-octanol, vortexed for 30 s, and then centrifuged to separate the phases. LB was added the broken droplet mixture and cells were plated onto agar plates with appropriate antibiotics. Sorted and unsorted cell plates were separately scraped the following day from plates, and reinoculated into LB with appropriate antibiotics for overnight culture. Minipreps of sorted and unsorted cultures were completed to extract DNA representative of the population as a whole. Double digest of the mixed plasmid population shows that a differential in the quantity of plasmid from pNAR_{low} vs pNAR_{high} can be identified upon a double restriction enzyme digestion of NdeI and XhoI (See Theoretical Digestion in FIG. 13, left). Equal volumes/concentrations of DNA from the sorted and unsorted minipreps were subjected to a double digest, where 30 μL of 95 ng/μL miniprepped DNA was mixed with 0.6 U/pL of each enzyme in 1× CutSmart buffer in a final volume of 35 μL for 3 hours at 37° C., followed by 65° C. for 20 minutes and a 12° C. hold. The digested DNA was then run on a 1% agarose gel at 90 V for 60 minutes stained with SybrSafe and imaged (See Experimental Digestion in FIG. 13, right). We observed a >8-fold enrichment of pathway pNAR_{high} cells by densitometry when comparing the presence of pNAR_{high} bands vs pNAR_{low} bands in the sorted vs unsorted population.

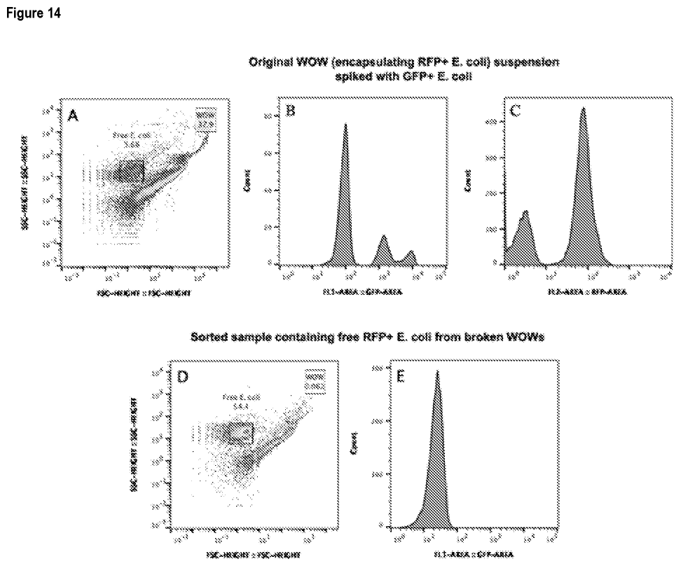

FIG. 14A, FIG. 14B, FIG. 14C, FIG. 14D, and FIG. 14E show sorting of double emulsion WOW droplets. A flow-focusing microfluidic device was used to generate single emulsion encapsulating E. coli cells (P674, RFP positive) of an initial OD600 0.75 A. The aqueous flow rate was 6.5 μl/min and the outer oil flow rate was 30 p/min. The geometry at the cross section of the device was 10 μm wide and 50 μm high. The average size of the water-in-oil (WO) droplets was 30 μm in diameter. Then the creamy layer of single emulsion was loaded into a 1 mL BD plastic syringe and re-injected into another flow-focusing microfluidic device at a flow rate of 2 μl/min. The outer aqueous phase, which was made of LB+KAN culture medium supplemented with 3% PVA, was run at a flow rate of 30 μl/min to pinch off water-in-oil-in-water (WOW) droplets at the flow-focusing section which was 35 um wide and 35 um high. The average size of the WOW droplets was 40 μm in diameter. The generated WOW droplets sank to the bottom of the collection tube covered by LB+KAN culture medium with 3% PVA.

To show that WOWs can be sorted away from the non-encapulated E coli which are generated during WOW productions, GFP expressing E coli were added to the WOW suspension.

In FIG. 14A, the forward scatter vs side scatter plot shows three distinct populations based on size. Free E coli near the origin represent 3.7% of the displayed events, WOWs near the axes maxima represent 38% of displayed events, and a broad population of sizes of WOW debris in between. FIG. 14B shows the GFP channel fluorescent response of the E coli sized population from FIG. 14A. Due to crosstalk between the channels, RFP positive E coli show as a population at about 75 fluorescent units. The GFP positive E coli added to the WOW population show at about 1000-10000 fluorescent units. FIG. 14C shows the RFP channel fluorescence of WOW sized events from FIG. 14A. RFP E coli containing WOWs show as a population at about 90 fluorescent units. Dark WOWs or Oil only droplets of the same size as the WOWs show as a population at about 2 fluorescent units. 10,000 WOW events were sorted from the population post sort population is shown in FIG. 14D. Note that the WOW droplets are destroyed by the sorting process as indicated by the presence of 0.1% of events. Free E coli represent 54% of observed events post sorting. FIG. 14E shows the GFP channel fluorescent response of the E coli sized population from FIG. 14D. Note that only RFP cells are observed on the axis. This demonstrates that while the WOWs did not survive the FACS sorting process, only RFP containing E coli from the WOWs were sorted into the collection tube.

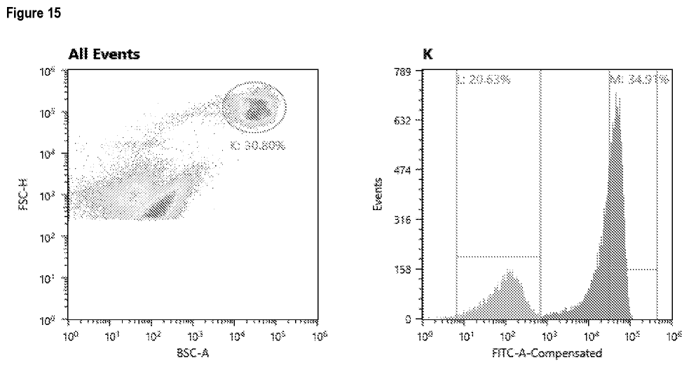

Alternatively to breaking the droplets with 1H,1H,2H,2H-Perfluoro-1-octanol and sorting individual cell events, single emulsion droplets can be encapsulated in a second aqueous phase to form double emulsions and analyzed/sorted on a standard FACS. FIG. 15 demonstrates analysis of mixed 2E6 pNAR_{high} pSENSOR_{GFP} and 2E6 pNARₙᵤₗₗ pSENSOR_{GFP} double emulsion droplets. Overnight LB pre-cultures of the two strains were washed and resuspended in filtered minimal glucose to a cell density of OD 0.04, which was used to ensure high occupancy. Single emulsion droplets were generated for 2E6 pNAR_{high} pSENSOR_{GFP} and 2E6 pNARₙᵤₗₗ pSENSOR_{GFP} separately using the protocol described in Example 4. After formation, the two droplet sets were incubated at 30° C. with tumbling for 18 h, at which point they were mixed and used to generate double emulsions, also as described in Example 4. FACS analysis of the double emulsions shows two distinct GFP (FITC-A-Compensated) populations within the gated double emulsion events (gate K, identified based on size), consistent with analysis of a mixed high producer and non-producer population.

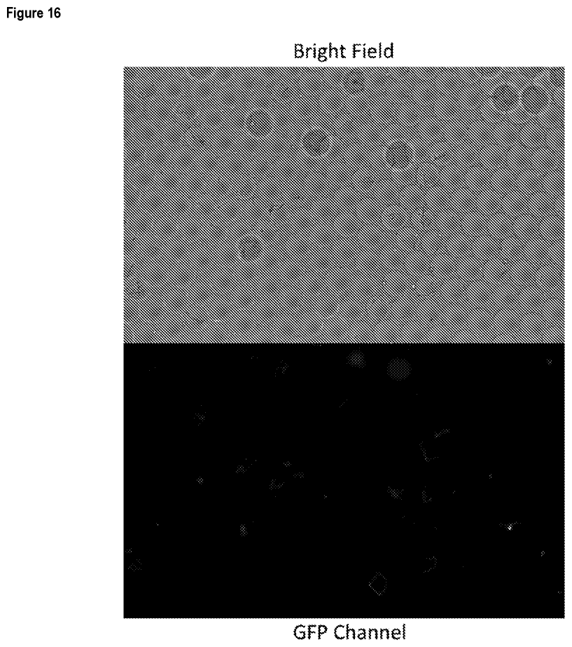

In addition to the use of typical fluorosurfactants (usually proprietary molecules/polymers/block copolymers), nanoparticle based pickering emulsions can also serve to encapsulate water in oil and oil in water droplets. FIG. 16 demonstrates that water in oil pickering emulsions can also be utilized with a producer+correlated sensor system to obtain a fluorescent signal. A 2E6 pNAR_{high} pSENSOR_{GFP} overnight culture from LB Medium plus appropriate antibiotics was washed three times in equal volume and resuspended in filtered 2×M9, 1% glucose, 1 mM IPTG, 0.1% pluronic F68, and appropriate antibiotics, such that the final resuspension OD₆₀₀ was 0.008 (as per prior empirical Poisson distribution determinations) for use as the single cell droplet loading aqueous phase. Droplets were generated using a 50 μm depth single aqueous stream droplet generator chip at a 20 μL/min flowrate for the pickering emulsion solution (Fluorophase, manufactured by Dolomite), and a 12 μL/min flowrate for the cell containing aqueous phase, generating droplets of 35-40 μm diameter. Droplets were incubated in an orbital shaker at 33° C. for 24 hours. The following day, droplets were imaged to observe the cellular characteristics/droplet occupancy. Results indicate that cells were capable of both proliferation (more than a single cell per occupied droplet, bright field) and production of fluorescent reporter (GFP channel) associated with pathway molecule production.

### EQUIVALENTS

All of the numerical ranges, amounts, values and percentages, such as those for amounts of materials, elemental contents, times and temperatures of reaction, ratios of amounts, and others, in the following portion of the specification and attached claims may be read as if prefaced by the word “about” even though the term “about” may not expressly appear with the value, amount, or range. Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contains error necessarily resulting from the standard deviation found in its underlying respective testing measurements. Furthermore, when numerical ranges are set forth herein, these ranges are inclusive of the recited range end points (e.g., end points may be used). When percentages by weight are used herein, the numerical values reported are relative to the total weight.

Also, it should be understood that any numerical range recited herein is intended to include all sub-ranges subsumed therein. For example, a range of “1 to 10” is intended to include all sub-ranges between (and including) the recited minimum value of 1 and the recited maximum value of 10, that is, having a minimum value equal to or greater than 1 and a maximum value of equal to or less than 10. The terms “one,” “a,” or “an” as used herein are intended to include “at least one” or “one or more,” unless otherwise indicated.

Any aspect or embodiment disclosed herein can be combined with any other aspect or embodiment as disclosed herein.

Any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

While this invention has been particularly shown and described with references to preferred embodiments thereof, it will be understood by those skilled in the art that various changes in form and details may be made therein without departing from the scope of the invention encompassed by the appended claims.

What is claimed is:

1. A method for producing a population of engineered producer cells comprising:
encapsulating each producer cell from a pool of genetically varied producer cells in a droplet to form a plurality of droplets encapsulating engineered producer cells;
assaying the droplets for levels of a target molecule, wherein an engineered protein-based sensor provides a readout of the level of the desired target molecule produced by the producer cell through activation or repression of a reporter;
isolating droplets with producer cells that produce desired levels of the target molecule; and,
recovering the cells that produce desired levels of the target molecule to form the population of producer cells, wherein the population of producer cells is an enriched population that produce desired levels of the target molecule.

2. The method of claim 1, wherein the recovery comprises:
(a) breaking the droplets,
(b) sorting the genetically varied producer cells, and
(c) growing the the producer cells on a growth medium.

3. The method of claim 2, wherein the sorting is by fluorescence activated droplet sorting (FADS) or fluorescence activated cell sorting (FACS).

4. The method of claim 2 or 3, wherein the recovery comprises:
(a) sorting the droplets,
(b) sorting the genetically varied producer cells, and
(c) growing the the producer cells on a growth medium.

5. The method of claim 4, wherein the sorting is by fluorescence activated droplet sorting (FADS) or fluorescence activated cell sorting (FACS).

6. The method of any one of claims 2-5, wherein breaking the droplets comprises breaking the droplets encapsulating isolated engineered producer cells that produce desired levels of the target molecule to form the population of engineered producer cells, wherein the population of engineered producer cells is an enriched population of engineered producer cells that produce desired levels of the target molecule.

7. The methods of any one of claims 1-6, wherein the DNA encoding the engineered protein-based sensor is encoded episomally.

8. The method of claim 7, wherein the DNA encoding the engineered protein-based sensor is encoded on a plasmid.

9. The methods of any one of claims 1-6, wherein the DNA encoding the engineered protein-based sensor is integrated in the genome of the producer cell.

10. The methods of any one of claims 1-9, wherein the engineered protein-based sensor is or has been transfected, transduced, transformed, or otherwise made available inside the producer cells.

11. The methods of any one of claims 1-10, wherein the reporter is a gene encoding a detectable marker that is activated in trans by the sensor-based protein.

12. The method of claim 11, wherein the detectable marker is an enzyme or a selectable marker.

13. The method of claim 12, wherein the enzyme is selected from lacZ, luciferase, or alkaline phosphatase.

14. The method of claim 12, wherein the selectable marker is an auxotroph, antibiotic, resistance marker, a toxin, or a spectrally detectable gene product.

15. The method of claim 12, wherein the selectable marker is a fluorescent protein.

16. The method of claim 14, wherein the spectrally detectable gene product is detected by spectroscopy or spectrometry.

17. The method of claim 11, wherein the gene encoding the reporter is encoded episomally.

18. The method of claim 17, wherein the gene encoding the reporter is encoded episomally on a plasmid.

19. The method of claim 18, wherein the gene encoding the reporter is encoded on the same plasmid as the gene encoding the engineered protein-based sensor.

20. The method of any one of claims 11-18, wherein the gene encoding the reporter is integrated in the genome.

21. The method of any one of claims 1-20, further comprising producing an engineered producer strain library from which the pool of engineered producer cells is taken, wherein the engineered producer strain library is engineered to produce one or more target molecules.

22. The methods of any one of claims 1-21, wherein engineered producer strain library is generated through genomic diversifying technology selected from multiplex automated genome-engineering (MAGE), plasmid-based production variation, or by non-GMO methods, wherein non-GMO methods are selected from chemical mutagenesis, radiation, and transposons.

23. The methods of any one of claims 1-22, wherein the droplets encapsulating isolated engineered producer cells further comprise growth medium and any required inducing agents.

24. The methods of any one of claims 1-23, wherein the readout level provided by the engineered protein sensor is by a reporter.

25. The method of claim 24, wherein the reporter is GFP.

26. The method of any one of claims 1-25, wherein the engineered protein sensor is a transcription factor.

27. The method of any one of claims 1-26, wherein the transcription factor is an allosteric transcription factor (aTF).

28. The method of any one of claims 1-27, wherein the engineered protein sensor is an engineered prokaryotic transcriptional regulator family member selected from a LysR, AraC/XylS, TetR, LuxR, LacI, ArsR, MerR, AsnC, MarR, NtrC (EBP), OmpR, DeoR, Cold shock, GntR, and Crp family member.

29. The method of any one of claims 1-27, wherein the engineered protein sensor is an engineered aTF listed in Table 1 (aTF (“Chassis”).

30. The method of any one of claims 1-29, wherein the target molecule is selected from the target molecules listed in Table 1 (Target Molecule Property).

31. A method for producing a population of engineered producer cells comprising:
transforming a pool of engineered producer cells with an engineered sensor plasmid, wherein the engineered sensor plasmid encodes an engineered protein sensor;
encapsulating each producer cell from the pool of genetically varied producer cells in a droplet to form a plurality of droplets encapsulating engineered producer cells;
assaying the droplets for levels of a target molecule, wherein an engineered protein-based sensor provides a readout of the level of the target molecule produced by the producer cell through activation or repression of a reporter;
isolating the droplets with producer cells that produce desired levels of the target molecule; and,
recovering the cells that produce desired levels of the target molecule to form the population of producer cells, wherein the population of producer cells is an enriched population that produce desired levels of the target molecule.

32. The method of claim 31, wherein the recovery comprises:
(a) breaking the droplets,
(b) sorting the genetically varied producer cells, and
(c) growing the the producer cells on a growth medium.

33. The method of claim 32, wherein the sorting is by fluorescence activated droplet sorting (FADS) or fluorescence activated cell sorting (FACS).

34. The method of claim 33, wherein the recovery comprises:
(a) sorting the droplets,
(b) sorting the genetically varied producer cells, and
(c) growing the the producer cells on a growth medium.

35. The method of claim 34, wherein the sorting is by fluorescence activated droplet sorting (FADS) or fluorescence activated cell sorting (FACS).

36. The method of any one of claims 32-35, wherein breaking the droplets comprises breaking the droplets encapsulating isolated engineered producer cells that produce desired levels of the target molecule to form the population of engineered producer cells, wherein the population of engineered producer cells is an enriched population of engineered producer cells that produce desired levels of the target molecule.

37. The methods of any one of claims 31-36, wherein the DNA encoding the engineered protein-based sensor is encoded episomally.

38. The method of claim 37, wherein the DNA encoding the engineered protein-based sensor is encoded on a plasmid.

39. The methods of any one of claims 31-36, wherein the DNA encoding the engineered protein-based sensor is integrated in the genome of the producer cell.

40. The methods of any one of claims 31-39, wherein the engineered protein-based sensor is or has been transfected, transduced, transformed, or otherwise made available inside the producer cells.

41. The methods of any one of claims 31-40, wherein the reporter is a gene encoding a detectable marker that is activated in trans by the sensor-based protein.

42. The method of claim 41, wherein the detectable marker is an enzyme or a selectable marker.

43. The method of claim 42, wherein the enzyme is selected from lacZ, luciferase, or alkaline phosphatase.

44. The method of claim 42, wherein the selectable marker is an auxotroph, antibiotic, resistance marker, a toxin, or a spectrally detectable gene product.

45. The method of claim 42, wherein the selectable marker is a fluorescent protein.

46. The method of claim 44, wherein the spectrally detectable gene product is detected by spectroscopy or spectrometry.

47. The method of claim 41, wherein the gene encoding the reporter is encoded episomally.

48. The method of claim 47, wherein the gene encoding the reporter is encoded episomally on a plasmid.

49. The method of claim 48, wherein the gene encoding the reporter is encoded on the same plasmid as the gene encoding the engineered protein-based sensor.

50. The method of any one of claims 41-48, wherein the gene encoding the reporter is integrated in the genome.

51. The method of any one of claims 31-50, further comprising producing an engineered producer strain library from which the pool of engineered producer cells is taken, wherein the engineered producer strain library is engineered to produce one or more target molecules.

52. The method of claim 51, wherein the engineered producer strain library is produced before transforming the pool of engineered producer cells with an engineered sensor plasmid.

53. The method of claim 51, wherein the engineered producer strain library is produced after transforming the pool of engineered producer cells with an engineered sensor plasmid.

54. The method of any one of claims 31-53, wherein engineered producer strain library is generated through genomic diversifying technology selected from multiplex automated genome-engineering (MAGE), plasmid-based production variation, or by non-GMO methods, wherein non-GMO methods are selected from chemical mutagenesis, radiation, and transposons.

55. The methods of any one of claims 31-54, wherein the droplets encapsulating isolated engineered producer cells further comprise growth medium and any required inducing agents.

56. The methods of any one of claims 31-55, wherein the readout level provided by the engineered protein sensor is by a reporter.

57. The method of claim 56, wherein the reporter is GFP.

58. The method of any one of claims 31-57, wherein the engineered protein sensor is a transcription factor.

59. The method of any one of claims 31-58, wherein the transcription factor is an allosteric transcription factor (aTF).

60. The method of any one of claims 31-59, wherein the engineered protein sensor is an engineered prokaryotic transcriptional regulator family member selected from a LysR, AraC/XylS, TetR, LuxR, LacI, ArsR, MerR, AsnC, MarR, NtrC (EBP), OmpR, DeoR, Cold shock, GntR, and Crp family member.

61. The method of any one of claims 31-58, wherein the engineered protein sensor is an engineered aTF listed in Table 1 (aTF (“Chassis”).

62. The method of any one of claims 31-61, wherein the target molecule is selected from the target molecules listed in Table 1 (Target Molecule Property).

63. A method for producing a population of engineered producer cells comprising:
encapsulating each producer cell from the pool of genetically varied producer cells in a droplet to form a plurality of droplets encapsulating engineered producer cells;
merging each droplet containing the producer cell with a droplet encapsulating an engineered-protein based sensor cell, wherein the engineered sensor cell produces an engineered protein sensor;
assaying the droplets for levels of a target molecule, wherein an engineered protein-based sensor provides a readout of the level of the desired target molecule produced by the producer cell through activation or repression of a reporter;
sorting the merged droplets to isolate droplets containing producer cells that produce desired levels of the target molecule; and,
recovering the cells that produce desired levels of the target molecule to form the population of producer cells, wherein the population of producer cells is an enriched population that produce desired levels of the target molecule.

64. The method of claim 63, wherein the recovery comprises:
(a) breaking the droplets,
(b) sorting the genetically varied producer cells, and
(c) growing the the producer cells on a growth medium.

65. The method of claim 64, wherein the sorting is by fluorescence activated droplet sorting (FADS) or fluorescence activated cell sorting (FACS).

66. The method of claim 65, wherein the recovery comprises:
(a) sorting the droplets,
(b) sorting the genetically varied producer cells, and
(c) growing the the producer cells on a growth medium.

67. The method of claim 66, wherein the sorting is by fluorescence activated droplet sorting (FADS) or fluorescence activated cell sorting (FACS).

68. The method of any one of claims 64-67, wherein breaking the droplets comprises breaking the droplets encapsulating isolated engineered producer cells that produce desired levels of the target molecule to form the population of engineered producer cells, wherein the population of engineered producer cells is an enriched population of engineered producer cells that produce desired levels of the target molecule.

69. The methods of any one of claims 63-68, wherein the DNA encoding the engineered protein-based sensor is encoded episomally.

70. The method of claim 69, wherein the DNA encoding the engineered protein-based sensor is encoded on a plasmid.

71. The methods of any one of claims 63-78, wherein the DNA encoding the engineered protein-based sensor is integrated in the genome of the producer cell.

72. The methods of any one of claims 1-71, wherein the engineered protein-based sensor is or has been transfected, transduced, transformed, or otherwise made available inside the producer cells.

73. The methods of any one of claims 1-72, wherein the reporter is a gene encoding a detectable marker that is activated in trans by the sensor-based protein.

74. The method of claim 73, wherein the detectable marker is an enzyme or a selectable marker.

75. The method of claim 74, wherein the enzyme is selected from lacZ, luciferase, or alkaline phosphatase.

76. The method of claim 74, wherein the selectable marker is an auxotroph, antibiotic, resistance marker, a toxin, or a spectrally detectable gene product.

77. The method of claim 74, the selectable marker is a fluorescent protein.

78. The method of claim 76, wherein the spectrally detectable gene product is detected by spectroscopy or spectrometry.

79. The method of claim 73, wherein the gene encoding the reporter is encoded episomally.

80. The method of claim 79, wherein the gene encoding the reporter is encoded episomally on a plasmid.

81. The method of claim 80, wherein the gene encoding the reporter is encoded on the same plasmid as the gene encoding the engineered protein-based sensor.

82. The method of any one of claims 73-80, wherein the gene encoding the reporter is integrated in the genome.

83. The method of any one of claims 63-82, further comprising producing an engineered producer strain library from which the pool of engineered producer cells is taken, wherein the engineered producer strain library is engineered to produce one or more target molecules.

84. The methods of any one of claims 63-83, wherein engineered producer strain library is generated through genomic diversifying technology selected from multiplex automated genome-engineering (MAGE), plasmid-based production variation, or by non-GMO methods, wherein non-GMO methods are selected from chemical mutagenesis, radiation, and transposons.

85. The methods of any one of claims 63-84, wherein the droplets encapsulating isolated engineered producer cells further comprise growth medium and any required inducing agents.

86. The methods of any one of claims 63-85, wherein the readout level provided by the engineered protein sensor is by a reporter.

87. The method of claim 86, wherein the reporter is GFP.

88. The method of any one of claims 63-87, wherein the engineered protein sensor is a transcription factor.

89. The method of any one of claims 63-88, wherein the transcription factor is an allosteric transcription factor (aTF).

90. The method of any one of claims 63-89, wherein the engineered protein sensor is an engineered prokaryotic transcriptional regulator family member selected from a LysR, AraC/XylS, TetR, LuxR, LacI, ArsR, MerR, AsnC, MarR, NtrC (EBP), OmpR, DeoR, Cold shock, GntR, and Crp family member.

91. The method of any one of claims 63-89, wherein the engineered protein sensor is an engineered aTF listed in Table 1 (aTF (“Chassis”).

92. The method of any one of claims 63-91, wherein the target molecule is selected from the target molecules listed in Table 1 (Target Molecule Property).

93. A method for producing a population of engineered producer cells comprising:
transforming a pool of engineered producer cells with an engineered sensor plasmid, wherein the engineered sensor plasmid encodes an engineered protein sensor;
encapsulating each producer cell from a pool of genetically varied producer cells in a droplet to form a plurality of droplets encapsulating engineered producer cells;
wherein each droplet is surrounded by an immiscible continuous phase that comprises a fluorinated-based oil or emulsion;
assaying the droplets for levels of a target molecule, wherein an engineered protein-based sensor provides a readout of the level of the desired target molecule produced by the producer cell through activation or repression of a reporter;
isolating droplets with producer cells that produce desired levels of the target molecule;
recovering the cells that produce desired levels of the target molecule to form the population of producer cells, wherein the population of producer cells is an enriched population that produce desired levels of the target molecule.

94. A method for producing a population of engineered producer cells comprising:
encapsulating each producer cell from the pool of genetically varied producer cells in a droplet to form a plurality of droplets encapsulating engineered producer cells; wherein each droplet is:
(a) surrounded by an immiscible continuous phase that comprises a fluorinated-based oil or emulsion, and
(b) comprises an engineered sensor cell, wherein the engineered sensor cell produces an engineered protein sensor;
assaying the droplets for levels of a target molecule, wherein an engineered protein-based sensor provides a readout of the level of the target molecule produced by the producer cell through activation or repression of a reporter;
isolating the droplets with producer cells that produce desired levels of the target molecule;
recovering the cells that produce desired levels of the target molecule to form the population of producer cells, wherein the population of producer cells is an enriched population that produce desired levels of the target molecule.

95. A method for producing a population of engineered producer cells comprising:
encapsulating each producer cell from the pool of genetically varied producer cells in a droplet to form a plurality of droplets encapsulating engineered producer cells;
wherein each droplet is surrounded by an immiscible continuous phase that comprises a fluorinated-based oil or emulsion;
merging each droplet containing the producer cell with a droplet encapsulating an engineered-protein based sensor cell, wherein the engineered sensor cell produces an engineered protein sensor;
assaying the droplets for levels of a target molecule, wherein an engineered protein-based sensor provides a readout of the level of the desired target molecule produced by the producer cell through activation or repression of a reporter;
sorting the merged droplets to isolate droplets containing producer cells that produce desired levels of the target molecule; and,
recovering the cells that produce desired levels of the target molecule to form the population of producer cells, wherein the population of producer cells is an enriched population that produce desired levels of the target molecule.

96. The method of any one claims 93-95, wherein the fluorinated-based oil or emulsion is an organic oil, a fluorinated oil, a fluorinated polymer, a water-in fluorocarbon emulsion, a water-in perfluorocarbon emulsion, or combinations thereof.

97. The method of any one claims 93-96, wherein the fluorinated-based oil or emulsion is stabilized by a particle.

98. The method of claim 97, wherein the particle is a partially fluorinated nanoparticle or a partially hydrophobic nanoparticle.

99. The method of claim 98, wherein the partially fluorinated nanoparticle or partially hydrophobic nanoparticle is a silica-based nanoparticle.

100. The method of any one claims 93-99, wherein the droplet is under microfluidic control.

101. The methods of any one of claims 93-100, further comprising producing an engineered producer strain library from which the pool of engineered producer cells is taken, wherein the engineered producer strain library is engineered to produce one or more target molecules.

102. The method of claim 101, wherein engineered producer strain library is generated through genomic diversifying technology selected from multiplex automated genome-engineering (MAGE), plasmid-based production variation, or by non-GMO methods, wherein non-GMO methods are selected from chemical mutagenesis, radiation, and transposons.

103. The methods of any one of claims 93-102, wherein the droplets encapsulating isolated engineered producer cells further comprise growth medium and any required inducing agents.

104. The methods of any one of claims 93-103, wherein the readout level provided by the engineered protein sensor is by a reporter.

105. The method of claim 104, wherein the reporter is GFP.

106. The method of any one of claims 93-105, wherein the engineered protein sensor is a transcription factor.

107. The method of any one of claims 93-106, wherein the transcription factor is an allosteric transcription factor (aTF).

108. The method of any one of claims 93-107, wherein the engineered protein sensor is an engineered prokaryotic transcriptional regulator family member selected from a LysR, AraC/XylS, TetR, LuxR, LacI, ArsR, MerR, AsnC, MarR, NtrC (EBP), OmpR, DeoR, Cold shock, GntR, and Crp family member.

109. The method of any one of claims 93-108, wherein the engineered protein sensor is an engineered aTF listed in Table 1.

110. The method of any one of claims 93-109, wherein the target molecule is selected from the target molecules listed in Table 1 (Target Molecule Property).

111. The method of any one of claims 93-110, wherein the recovery comprises:
(a) breaking the droplets,
(b) sorting the genetically varied producer cells, and
(c) growing the the producer cells on a growth medium.

112. The method of claim 111, wherein the sorting is by fluorescence activated droplet sorting (FADS) or fluorescence activated cell sorting (FACS).

113. The method of any one of claims 93-112, wherein the recovery comprises:
(a) sorting the droplets,
(b) sorting the genetically varied producer cells, and
(c) growing the the producer cells on a growth medium.

114. The method of claim 113, wherein the sorting is by fluorescence activated droplet sorting (FADS) or fluorescence activated cell sorting (FACS).

115. The method of any one of claims 111-114, wherein breaking the droplets comprises breaking the droplets encapsulating isolated engineered producer cells that produce desired levels of the target molecule to form the population of engineered producer cells, wherein the population of engineered producer cells is an enriched population of engineered producer cells that produce desired levels of the target molecule.

116. The methods of any one of claims 93-115, wherein the DNA encoding the engineered protein-based sensor is encoded episomally.

117. The method of claim 116, wherein the DNA encoding the engineered protein-based sensor is encoded on a plasmid.

118. The methods of any one of claims 93-115, wherein the DNA encoding the engineered protein-based sensor is integrated in the genome of the producer cell.

119. The methods of any one of claims 93-118, wherein the engineered protein-based sensor is or has been transfected, transduced, transformed, or otherwise made available inside the producer cells.

120. The methods of any one of claims 93-119, wherein the reporter is a gene encoding a detectable marker that is activated in trans by the sensor-based protein.

121. The method of claim 120, wherein the detectable marker is an enzyme or a selectable marker.

122. The method of claim 121, wherein the enzyme is selected from lacZ, luciferase, or alkaline phosphatase.

123. The method of claim 122, wherein the selectable marker is an auxotroph, antibiotic, resistance marker, a toxin, or a spectrally detectable gene product.

124. The method of claim 120, wherein the selectable marker is a fluorescent protein.

125. The method of claim 124, wherein the spectrally detectable gene product is detected by spectroscopy or spectrometry.

126. The method of claim 120, wherein the gene encoding the reporter is encoded episomally.

127. The method of claim 126, wherein the gene encoding the reporter is encoded episomally on a plasmid.

128. The method of claim 127, wherein the gene encoding the reporter is encoded on the same plasmid as the gene encoding the engineered protein-based sensor.

129. The method of any one of claims 120-126, wherein the gene encoding the reporter is integrated in the genome.

130. The method of any one of claims 1-30, wherein the engineered protein-based sensor and reporter are encoded within the producer cell.

131. The method of any one of claims 1-30, wherein the engineered protein-based sensor and reporter are encoded within a co-encapsulated sensor cell.

132. The method of any one of claims 1-30, wherein the engineered protein-based sensor and reporter are encoded within a sensor cell which is encapsulated in a separate droplet, which is then merged with the droplet containing an engineered producer cell.

133. The method of any one of claims 31-62, wherein the engineered protein-based sensor and reporter are encoded within the producer cell.

134. The method of any one of claims 31-62, wherein the engineered protein-based sensor and reporter are encoded within a co-encapsulated sensor cell.

135. The method of any one of claims 31-62, wherein the engineered protein-based sensor and reporter are encoded within a sensor cell which is encapsulated in a separate droplet, which is then merged with the droplet containing an engineered producer cell.

136. The method of any one of claims 63-92, wherein the engineered protein-based sensor and reporter are encoded within the producer cell.

137. The method of any one of claims 63-92, wherein the engineered protein-based sensor and reporter are encoded within a co-encapsulated sensor cell.

138. The method of any one of claims 63-92, wherein the engineered protein-based sensor and reporter are encoded within a sensor cell which is encapsulated in a separate droplet, which is then merged with the droplet containing an engineered producer cell.

139. The method of any one of claims 93-129, wherein the engineered protein-based sensor and reporter are encoded within the producer cell.

140. The method of any one of claims 93-129, wherein the engineered protein-based sensor and reporter are encoded within a co-encapsulated sensor cell.

141. The method of any one of claims 93-129, wherein the engineered protein-based sensor and reporter are encoded within a sensor cell which is encapsulated in a separate droplet, which is then merged with the droplet containing an engineered producer cell.
